SEQUENCE LISTING (10 sequences)

SEQ ID NO: 1 - DNA, 2190 bp, Homo sapiens
  source 1..2190: mol_type = unassigned DNA
atgggggattacgggtttggagtgctagtgcaaagcaatactgggaataaatctgcttttccagtcagattccatccacatctgcagcctccacaccatcaccaaaatgccacccccagccctgctgcttttataaataataacacagctgccaatggcagcagtgctgggtcagcttggctttttcctgctccagctacccataacattcaggatgagatcttggggtcagaaaaagcaaaaagtcagcaacaggaacagcaagaccccttagaaaagcagcagctttccccaagtccaggtcaggaagctggaatactgcctgaaacagagaaggcaaaatcagaagaaaatcaaggggacaattcttcggaaaatggcaatgggaaggagaaaataaggatcgaatctccagtgttgacagggtttgattatcaagaagccactgggctaggtacttcaacccaacccttgacatctagcgcatcgtctcttactggtttcagtaactggtcagcagcgatagcgccttcctcctctacaataatcaatgaagatgcaagtttctttcaccagggaggggtccctgctgcttcggctaataacggtgctctgttgtttcaaaatttcccccatcatgtcagccctggctttggaggcagcttctctcctcagatcgggcctctctcacagcaccacccacatcaccctcatttccagcatcatcacagccagcatcagcagcaaaggaggtctcctgccagtccccatcccccacccttcacacatagaaatgctgcttttaaccagctgcctcatttggcgaataatcttaacaaacccccctctccgtggagcagctaccagagtccgtcaccaacaccctcctcttcctggagcccgggcggtggtggatatggtggctggggaggttcccaaggccgagatcaccgcagagggctgaatggtggaataacgcccctgaactccatctcgcctttgaagaaaaattttgcaagcaatcatattcagctccagaagtatgctcgccccagctctgcctttgcacctaaatcctggatggaagatagcttgaacagggctgacaacatttttccttttccggatcgccccaggacattcgacatgcactcactggagagttcactcattgacataatgagagctgaaaatgataccattaaaggtcgtctaaactattcatatccaggatccgatagctctctgcttattaatgcaaggacatatgggcgaaggagaggtcagtcttcactgtttccaatggaagatggattcttggatgatggccgtggggatcagcctcttcatagtggcctgggttcacctcactgcttcagtcaccagaatggggaaagagtggaacgatattctcgaaaggtgtttgtaggcggattgcctccagacattgatgaagatgagatcacagctagttttcgtcgctttggccctctgattgtggattggcctcataaagctgagagcaaatcctattttcctcctaaaggctatgcattcctgctgtttcaagatgaaagctctgtgcaggctctcattgatgcatgcattgaagaagatggaaaactctacctttgtgtatcaagtcccactatcaaggataagccagtccagattcggccttggaatctcagtgacagtgactttgtgatggatggttcacagccacttgacccacgaaaaactatatttgttggtggtgttcctcgaccattacgagctgtggagcttgcgatgataatggatcggctatatggaggtgtgtgctacgctgggattgataccgaccctgagctaaaatacccaaaaggagctgggagagttgcgttctctaatcaacagagttacatagctgctatcagtgcccgctttgttcagctgcagcatggagagatagataaacgggtggaagttaagccatatgtcttggatgatcagctgtgtgatgaatgtcagggggcccgttgtggggggaaatttgctccatttttctgtgctaatgttacctgtctgcagtattactgtgaatattgctgggctgctatccattctcgtgctggcagggaattccacaagcccctggtgaaggaaggcggtgaccgccctcggcatatttcattccgctggaactaa

SEQ ID NO: 2 - protein, 729 aa, Homo sapiens
MGDYGFGVLVQSNTGNKSAFPVRFHPHLQPPHHHQNATPSPAAFINNNTAANGSSAGSAWLFPAPATHNIQDEILGSEKAKSQQQEQQDPLEKQQLSPSPGQEAGILPETEKAKSEENQGDNSSENGNGKEKIRIESPVLTGFDYQEATGLGTSTQPLTSSASSLTGFSNWSAAIAPSSSTIINEDASFFHQGGVPAASANNGALLFQNFPHHVSPGFGGSFSPQIGPLSQHHPHHPHFQHHHSQHQQQRRSPASPHPPPFTHRNAAFNQLPHLANNLNKPPSPWSSYQSPSPTPSSSWSPGGGGYGGWGGSQGRDHRRGLNGGITPLNSISPLKKNFASNHIQLQKYARPSSAFAPKSWMEDSLNRADNIFPFPDRPRTFDMHSLESSLIDIMRAENDTIKGRLNYSYPGSDSSLLINARTYGRRRGQSSLFPMEDGFLDDGRGDQPLHSGLGSPHCFSHQNGERVERYSRKVFVGGLPPDIDEDEITASFRRFGPLIVDWPHKAESKSYFPPKGYAFLLFQDESSVQALIDACIEEDGKLYLCVSSPTIKDKPVQIRPWNLSDSDFVMDGSQPLDPRKTIFVGGVPRPLRAVELAMIMDRLYGGVCYAGIDTDPELKYPKGAGRVAFSNQQSYIAAISARFVQLQHGEIDKRVEVKPYVLDDQLCDECQGARCGGKFAPFFCANVTCLQYYCEYCWAAIHSRAGREFHKPLVKEGGDRPRHISFRWN

SEQ ID NO: 3 - DNA, 2139 bp, Homo sapiens
  source 1..2139: mol_type = unassigned DNA
atgggggattacgggtttggagtgctagtgcaaagcaatactgggaataaatctgcttttccagtcagattccatccacatctgcagcctccacaccatcaccaaaatgccacccccagccctgctgcttttataaataataacacagctgccaatggcagcagtgctgggtcagcttggctttttcctgctccagctacccataacattcaggatgagatcttggggtcagaaaaagcaaaaagtcagcaacaggaacagcaagaccccttagaaaagcagcagctttccccaagtccaggtcaggaagctggaatactgcctgaaacagagaaggcaaaatcagaagaaaatcaaggggacaattcttcggaaaatggcaatgggaaggagaaaataaggatcgaatctccagtgttgacagggtttgattatcaagaagccactgggctaggtacttcaacccaacccttgacatctagcgcatcgtctcttactggtttcagtaactggtcagcagcgatagcgccttcctcctctacaataatcaatgaagatgcaagtttctttcaccagggaggggtccctgctgcttcggctaataacggtgctctgttgtttcaaaatttcccccatcatgtcagccctggctttggaggcagcttctctcctcagatcgggcctctctcacagcaccacccacatcaccctcatttccagcatcatcacagccagcatcagcagcaaaggaggtctcctgccagtccccatcccccacccttcacacatagaaatgctgcttttaaccagctgcctcatttggcgaataatcttaacaaacccccctctccgtggagcagctaccagagtccgtcaccaacaccctcctcttcctggagcccgggcggtggtggatatggtggctggggaggttcccaaggccgagatcaccgcagagggctgaatggtggaataacgcccctgaactccatctcgcctttgaagaaaaattttgcaagcaatcatattcagctccagaagtatgctcgccccagctctgcctttgcacctaaatcctggatggaagatagcttgaacagggctgacaacatttttccttttccggatcgccccaggacattcgacatgcactcactggagagttcactcattgacataatgagagctgaaaatgataccattaaagcaaggacatatgggcgaaggagaggtcagtcttcactgtttccaatggaagatggattcttggatgatggccgtggggatcagcctcttcatagtggcctgggttcacctcactgcttcagtcaccagaatggggaaagagtggaacgatattctcgaaaggtgtttgtaggcggattgcctccagacattgatgaagatgagatcacagctagttttcgtcgctttggccctctgattgtggattggcctcataaagctgagagcaaatcctattttcctcctaaaggctatgcattcctgctgtttcaagatgaaagctctgtgcaggctctcattgatgcatgcattgaagaagatggaaaactctacctttgtgtatcaagtcccactatcaaggataagccagtccagattcggccttggaatctcagtgacagtgactttgtgatggatggttcacagccacttgacccacgaaaaactatatttgttggtggtgttcctcgaccattacgagctgtggagcttgcgatgataatggatcggctatatggaggtgtgtgctacgctgggattgataccgaccctgagctaaaatacccaaaaggagctgggagagttgcgttctctaatcaacagagttacatagctgctatcagtgcccgctttgttcagctgcagcatggagagatagataaacgggtggaagttaagccatatgtcttggatgatcagctgtgtgatgaatgtcagggggcccgttgtggggggaaatttgctccatttttctgtgctaatgttacctgtctgcagtattactgtgaatattgctgggctgctatccattctcgtgctggcagggaattccacaagcccctggtgaaggaaggcggtgaccgccctcggcatatttcattccgctggaactaa

SEQ ID NO: 4 - protein, 712 aa, Homo sapiens
MGDYGFGVLVQSNTGNKSAFPVRFHPHLQPPHHHQNATPSPAAFINNNTAANGSSAGSAWLFPAPATHNIQDEILGSEKAKSQQQEQQDPLEKQQLSPSPGQEAGILPETEKAKSEENQGDNSSENGNGKEKIRIESPVLTGFDYQEATGLGTSTQPLTSSASSLTGFSNWSAAIAPSSSTIINEDASFFHQGGVPAASANNGALLFQNFPHHVSPGFGGSFSPQIGPLSQHHPHHPHFQHHHSQHQQQRRSPASPHPPPFTHRNAAFNQLPHLANNLNKPPSPWSSYQSPSPTPSSSWSPGGGGYGGWGGSQGRDHRRGLNGGITPLNSISPLKKNFASNHIQLQKYARPSSAFAPKSWMEDSLNRADNIFPFPDRPRTFDMHSLESSLIDIMRAENDTIKARTYGRRRGQSSLFPMEDGFLDDGRGDQPLHSGLGSPHCFSHQNGERVERYSRKVFVGGLPPDIDEDEITASFRRFGPLIVDWPHKAESKSYFPPKGYAFLLFQDESSVQALIDACIEEDGKLYLCVSSPTIKDKPVQIRPWNLSDSDFVMDGSQPLDPRKTIFVGGVPRPLRAVELAMIMDRLYGGVCYAGIDTDPELKYPKGAGRVAFSNQQSYIAAISARFVQLQHGEIDKRVEVKPYVLDDQLCDECQGARCGGKFAPFFCANVTCLQYYCEYCWAAIHSRAGREFHKPLVKEGGDRPRHISFRWN

SEQ ID NO: 5 - DNA, 2115 bp, Homo sapiens
  source 1..2115: mol_type = unassigned DNA
atgggggattacgggtttggagtgctagtgcaaagcaatactgggaataaatctgcttttccagtcagattccatccacatctgcagcctccacaccatcaccaaaatgccacccccagccctgctgcttttataaataataacacagctgccaatggcagcagtgctgggtcagcttggctttttcctgctccagctacccataacattcaggatgagatcttggggtcagaaaaagcaaaaagtcagcaacaggaacagcaagaccccttagaaaagcagcagctttccccaagtccaggtcaggaagctggaatactgcctgaaacagagaaggcaaaatcagaagaaaatcaaggggacaattcttcggaaaatggcaatgggaaggagaaaataaggatcgaatctccagtgttgacagggtttgattatcaagaagccactgggctaggtacttcaacccaacccttgacatctagcgcatcgtctcttactggtttcagtaactggtcagcagcgatagcgccttcctcctctacaataatcaatgaagatgcaagtttctttcaccagggaggggtccctgctgcttcggctaataacggtgctctgttgtttcaaaatttcccccatcatgtcagccctggctttggaggcagcttctctcctcagatcgggcctctctcacagcaccacccacatcaccctcatttccagcatcatcacagccagcatcagcagcaaaggaggtctcctgccagtccccatcccccacccttcacacatagaaatgctgcttttaaccagctgcctcatttggcgaataatcttaacaaacccccctctccgtggagcagctaccagagtccgtcaccaacaccctcctcttcctggagcccgggcggtggtggatatggtggctggggaggttcccaaggccgagatcaccgcagagggctgaatggtggaataacgcccctgaactccatctcgcctttgaagaaaaattttgcaagcaatcatattcagctccagaagtatgctcgccccagctctgcctttgcacctaaatcctggatggaagatagcttgaacagggctgacaacatttttccttttccggatcgccccaggacattcgacatgcactcactggagagttcactcattgacataatgagagctgaaaatgataccattaaaggtcagtcttcactgtttccaatggaagatggattcttggatgatggccgtggggatcagcctcttcatagtggcctgggttcacctcactgcttcagtcaccagaatggggaaagagtggaacgatattctcgaaaggtgtttgtaggcggattgcctccagacattgatgaagatgagatcacagctagttttcgtcgctttggccctctgattgtggattggcctcataaagctgagagcaaatcctattttcctcctaaaggctatgcattcctgctgtttcaagatgaaagctctgtgcaggctctcattgatgcatgcattgaagaagatggaaaactctacctttgtgtatcaagtcccactatcaaggataagccagtccagattcggccttggaatctcagtgacagtgactttgtgatggatggttcacagccacttgacccacgaaaaactatatttgttggtggtgttcctcgaccattacgagctgtggagcttgcgatgataatggatcggctatatggaggtgtgtgctacgctgggattgataccgaccctgagctaaaatacccaaaaggagctgggagagttgcgttctctaatcaacagagttacatagctgctatcagtgcccgctttgttcagctgcagcatggagagatagataaacgggtggaagttaagccatatgtcttggatgatcagctgtgtgatgaatgtcagggggcccgttgtggggggaaatttgctccatttttctgtgctaatgttacctgtctgcagtattactgtgaatattgctgggctgctatccattctcgtgctggcagggaattccacaagcccctggtgaaggaaggcggtgaccgccctcggcatatttcattccgctggaactaa

SEQ ID NO: 6 - protein, 704 aa, Homo sapiens
MGDYGFGVLVQSNTGNKSAFPVRFHPHLQPPHHHQNATPSPAAFINNNTAANGSSAGSAWLFPAPATHNIQDEILGSEKAKSQQQEQQDPLEKQQLSPSPGQEAGILPETEKAKSEENQGDNSSENGNGKEKIRIESPVLTGFDYQEATGLGTSTQPLTSSASSLTGFSNWSAAIAPSSSTIINEDASFFHQGGVPAASANNGALLFQNFPHHVSPGFGGSFSPQIGPLSQHHPHHPHFQHHHSQHQQQRRSPASPHPPPFTHRNAAFNQLPHLANNLNKPPSPWSSYQSPSPTPSSSWSPGGGGYGGWGGSQGRDHRRGLNGGITPLNSISPLKKNFASNHIQLQKYARPSSAFAPKSWMEDSLNRADNIFPFPDRPRTFDMHSLESSLIDIMRAENDTIKGQSSLFPMEDGFLDDGRGDQPLHSGLGSPHCFSHQNGERVERYSRKVFVGGLPPDIDEDEITASFRRFGPLIVDWPHKAESKSYFPPKGYAFLLFQDESSVQALIDACIEEDGKLYLCVSSPTIKDKPVQIRPWNLSDSDFVMDGSQPLDPRKTIFVGGVPRPLRAVELAMIMDRLYGGVCYAGIDTDPELKYPKGAGRVAFSNQQSYIAAISARFVQLQHGEIDKRVEVKPYVLDDQLCDECQGARCGGKFAPFFCANVTCLQYYCEYCWAAIHSRAGREFHKPLVKEGGDRPRHISFRWN

SEQ ID NO: 7 - DNA, 1020 bp, Homo sapiens
  source 1..1020: mol_type = unassigned DNA
atgcactcactggagagttcactcattgacataatgagagctgaaaatgataccattaaaggtcgtctaaactattcatatccaggatccgatagctctctgcttattaatggtcagtcttcactgtttccaatggaagatggattcttggatgatggccgtggggatcagcctcttcatagtggcctgggttcacctcactgcttcagtcaccagaatggggaaagagtggaacgatattctcgaaaggtgtttgtaggcggattgcctccagacattgatgaagatgagatcacagctagttttcgtcgctttggccctctgattgtggattggcctcataaagctgagagcaaatcctattttcctcctaaaggctatgcattcctgctgtttcaagatgaaagctctgtgcaggctctcattgatgcatgcattgaagaagatggaaaactctacctttgtgtatcaagtcccactatcaaggataagccagtccagattcggccttggaatctcagtgacagtgactttgtgatggatggttcacagccacttgacccacgaaaaactatatttgttggtggtgttcctcgaccattacgagctgtggagcttgcgatgataatggatcggctatatggaggtgtgtgctacgctgggattgataccgaccctgagctaaaatacccaaaaggagctgggagagttgcgttctctaatcaacagagttacatagctgctatcagtgcccgctttgttcagctgcagcatggagagatagataaacgggtggaagttaagccatatgtcttggatgatcagctgtgtgatgaatgtcagggggcccgttgtggggggaaatttgctccatttttctgtgctaatgttacctgtctgcagtattactgtgaatattgctgggctgctatccattctcgtgctggcagggaattccacaagcccctggtgaaggaaggcggtgaccgccctcggcatatttcattccgctggaactaa

SEQ ID NO: 8 - protein, 339 aa, Homo sapiens
MHSLESSLIDIMRAENDTIKGRLNYSYPGSDSSLLINGQSSLFPMEDGFLDDGRGDQPLHSGLGSPHCFSHQNGERVERYSRKVFVGGLPPDIDEDEITASFRRFGPLIVDWPHKAESKSYFPPKGYAFLLFQDESSVQALIDACIEEDGKLYLCVSSPTIKDKPVQIRPWNLSDSDFVMDGSQPLDPRKTIFVGGVPRPLRAVELAMIMDRLYGGVCYAGIDTDPELKYPKGAGRVAFSNQQSYIAAISARFVQLQHGEIDKRVEVKPYVLDDQLCDECQGARCGGKFAPFFCANVTCLQYYCEYCWAAIHSRAGREFHKPLVKEGGDRPRHISFRWN

SEQ ID NO: 9 - DNA, 969 bp, Homo sapiens
  source 1..969: mol_type = unassigned DNA
atgcactcactggagagttcactcattgacataatgagagctgaaaatgataccattaaaggtcagtcttcactgtttccaatggaagatggattcttggatgatggccgtggggatcagcctcttcatagtggcctgggttcacctcactgcttcagtcaccagaatggggaaagagtggaacgatattctcgaaaggtgtttgtaggcggattgcctccagacattgatgaagatgagatcacagctagttttcgtcgctttggccctctgattgtggattggcctcataaagctgagagcaaatcctattttcctcctaaaggctatgcattcctgctgtttcaagatgaaagctctgtgcaggctctcattgatgcatgcattgaagaagatggaaaactctacctttgtgtatcaagtcccactatcaaggataagccagtccagattcggccttggaatctcagtgacagtgactttgtgatggatggttcacagccacttgacccacgaaaaactatatttgttggtggtgttcctcgaccattacgagctgtggagcttgcgatgataatggatcggctatatggaggtgtgtgctacgctgggattgataccgaccctgagctaaaatacccaaaaggagctgggagagttgcgttctctaatcaacagagttacatagctgctatcagtgcccgctttgttcagctgcagcatggagagatagataaacgggtggaagttaagccatatgtcttggatgatcagctgtgtgatgaatgtcagggggcccgttgtggggggaaatttgctccatttttctgtgctaatgttacctgtctgcagtattactgtgaatattgctgggctgctatccattctcgtgctggcagggaattccacaagcccctggtgaaggaaggcggtgaccgccctcggcatatttcattccgctggaactaa

SEQ ID NO: 10 - protein, 322 aa, Homo sapiens
MHSLESSLIDIMRAENDTIKGQSSLFPMEDGFLDDGRGDQPLHSGLGSPHCFSHQNGERVERYSRKVFVGGLPPDIDEDEITASFRRFGPLIVDWPHKAESKSYFPPKGYAFLLFQDESSVQALIDACIEEDGKLYLCVSSPTIKDKPVQIRPWNLSDSDFVMDGSQPLDPRKTIFVGGVPRPLRAVELAMIMDRLYGGVCYAGIDTDPELKYPKGAGRVAFSNQQSYIAAISARFVQLQHGEIDKRVEVKPYVLDDQLCDECQGARCGGKFAPFFCANVTCLQYYCEYCWAAIHSRAGREFHKPLVKEGGDRPRHISFRWN

(19) United States
(12) Patent Application Publication
(10) Pub. No.: US 2024/0358858 A1
(43) Pub. Date: Oct. 31, 2024

(54) COMPOSITIONS AND METHODS FOR ASSESSING AND TREATING AGE-RELATED CONDITIONS

(71) Applicant: The Hong Kong University of Science and Technology, Kowloon, Hong Kong (CN)

(72) Inventors: Hiu Tung Cheung, Happy Valley (CN); Wenshu Zeng, Hong Kong (CN)

(21) Appl. No.: 18/577,797

(22) PCT Filed: Aug. 26, 2022
(86) PCT No.: PCT/CN2022/115050

§ 371 Date: Jan. 9, 2024

Related U.S. Application Data
(60) Provisional application No. 63/237,147, filed on Aug. 26, 2021.

(51) Int. Cl. A61K 48/00; A61K 38/17; A61P 21/00; C12N 5/0775; G01N 33/50

(52) U.S. Cl. CPC A61K 48/0058; A61K 38/1709; A61P 21/00; C12N 5/0668; G01N 33/5061; G01N 2333/4704; G01N 2800/7042

(57) ABSTRACT

Compositions and methods are provided for the assessment and treatment of age-related conditions and diseases. Also provided are methods for identifying compounds that can potentially regulate the aging process.

### RELATED APPLICATIONS

This application claims priority to U.S. Provisional Patent Application No. 63/237,147, filed Aug. 26, 2021, the contents of which are hereby incorporated by reference in the entirety for all purposes.

### BACKGROUND OF THE INVENTION

The decline in tissue regenerative potential is a consequence of the aging process (Brack and Muñoz-Cánoves, 2015; Rando, 2006; Rando and Chang, 2012). In various adult tissues, age-dependent defects in tissue homeostasis or repair are correlated with altered stem cell function (Alt et al., 2012; Chandel et al., 2016; Conboy and Rando, 2005; Ge et al., 2020). In skeletal muscle, the number and function of muscle stem cells (also called satellite cells, SCs) decline during aging, resulting in impaired muscle regeneration after injury (García-Prat et al., 2016; Li et al., 2019; Sousa-Victor et al., 2014). The aged stem cell niche and dysregulation of cellular functions such as impaired autophagy and dysregulated transcriptomes correlate with SC death and the transition from reversible quiescence into pre-senescence (García-Prat et al., 2016; Liu et al., 2018). Genome stability is also affected during the aging process in SCs (Behrens et al., 2014; Mejia-Ramirez and Florian, 2020; Schultz and Sinclair, 2016). The major player for the regulation of cellular functions or maintenance is protein. However, the proteomic landscape of SCs during aging is unexamined.

SCs are kept in quiescence in resting muscle but are activated swiftly upon muscle injury followed by rapid myogenic lineage progression for muscle repair (Cheung and Rando, 2013; Cheung et al., 2012; Shi and Garry, 2006; So and Cheung, 2018). In response to injury or disease, SCs undergo rapid activation followed by robust proliferation to yield a pool of myogenic transcription factor Myod1 expressing progenitor cells (Yin et al., 2013; Yue et al., 2020; Zammit et al., 2006). Afterwards, progenitor cells express Myogenin and begin to differentiate to form newly regenerated muscle fibers. A subset of progenitor cells undergo self-renewal to replenish the quiescent SC population (Kuang et al., 2008; Motohashi and Asakura, 2014; Urbán and Cheung, 2021). During the aging process, the capacity of SCs to activate, proliferate, and differentiate is reduced (Brett et al., 2020; Li et al., 2019; Sousa-Victor et al., 2014). However, the mechanisms behind this decline remain elusive. Robust myogenic lineage progression requires sufficient energy supply (Bhattacharya and Scimè, 2020; Sala et al., 2019) of which mitochondria are the centers for energy metabolism (Cogliati et al., 2016; Friedman and Nunnari, 2014; Wallace et al., 2010). Yet, whether the mitochondrial proteome and activity are altered during aging remains largely unknown in SCs.

Cytoplasmic polyadenylation element binding proteins 1-4 (CPEB1-4) are RNA-binding proteins that bind to cytoplasmic polyadenylation element (CPE) sequences and regulate translation of its target transcripts by inducing cytoplasmic manipulation of their poly(A)-tails (Bava et al., 2013; Cao et al., 2018; Fernández-Miranda and Méndez, 2012; Ford et al., 2019; Lu et al., 2017a). CPEs, located in the 3′ UTRs, are found on approximately 20% of mammalian transcripts (Bava et al., 2013; Piqué et al., 2008). After binding to the CPE, CPEB proteins recruits cytoplasmic poly (A) polymerase GLD2 to elongate the poly (A) tail to maintain mRNA stability (Richter, 2007). mRNA stability positively correlates with translational output (Baek et al., 2008). CPEB1-4 expression is tissue-specific and binds to specific transcripts to regulate their corresponding cellular functions (Cao et al., 2018; Chen and Huang, 2012; Ford et al., 2019; Kochanek and Wells, 2013; Lu et al., 2017b; Parras et al., 2018; Zhang et al., 2020). The function of the CPEB protein family in SCs during aging is, however poorly understood.

In summary, age-associated impairments in stem cell function correlate with a decline in somatic tissue regeneration capacity following tissue injury or during disease. Altered cellular metabolism correlates with cellular senescence during aging. Therefore, methods of manipulating metabolism to rejuvenate aged cells are of great interest for anti-aging therapeutic applications. There is a distinct need to develop new and effective treatment methods for age-related conditions and illnesses. This invention fulfills this and other related needs.

### BRIEF SUMMARY OF THE INVENTION

The application provides the first disclosure of cytoplasmic polyadenylation element binding protein (CPEB) 4, a member of a family of RNA-binding proteins that bind to cytoplasmic polyadenylation element (CPE) sequences and regulate translation of their target transcripts by inducing cytoplasmic manipulation of the poly(A) tails of the transcripts, playing a role in the regulation of mitochondrial activities. Importantly, expression levels of CPEB4 fall during aging. New compositions and methods for treating aging-related diseases and disorders by increasing CPEB4 activity are therefore devised from this discovery.

As such, in a first aspect, this invention provides a method for rejuvenating a cell or reducing senescence in a cell. The method includes a step of increasing the expression level or activity level of a CPEB4 protein in the cell. In some embodiments, the cell is a muscle cell or muscle stem cell. In some embodiments, step (1) comprises introducing into the cell a nucleic acid encoding a CPEB4 protein such that an effective amount of the CPEB4 protein is expressed in the cell. In some embodiments, the cell is within a human body.

In a related aspect, the present invention provides a method for treating an age-related condition or disease in a human patient by way of administering to the human an expression vector comprising a polynucleotide sequence encoding a CPEB4 protein. In some embodiments, the expression vector is administered by subcutaneous, intramuscular, intravenous, or intraperitoneal injection, or by oral administration. In some embodiments, the step of increasing the expression level or activity level of a CPEB4 protein in the cell is performed ex vivo prior to the cell being reintroduced back into the human body. In some embodiments, the cell is a muscle stem cell and wherein the cell is reintroduced to a muscle injury site. In some embodiments, the expression vector comprises a muscle cell-specific promoter, for example, a Pax7 promoter, operably linked to a polynucleotide sequence encoding CPEB4 protein.

In a second aspect, the present invention provides rejuvenated cells produced by the methods described above and herein as well as compositions comprising such cells. In some embodiments, the cells are muscle cells or muscle stem cells. In some embodiments, the cells are present in a pharmaceutical or physiological composition that further comprises one or more pharmaceutically/physiologically acceptable excipients. In some embodiments, the composition is formulated for subcutaneous, intramuscular, intravenous, or intraperitoneal injection, or for oral administration. Kits are also provided, which comprise a container containing (a) a composition comprising an expression vector, which comprises a polynucleotide sequence encoding a CPEB4 protein; or (b) a composition comprising the rejuvenated cells produced by the methods described above and herein as well as compositions comprising the cells, such as muscle cells or muscle stem cells. In some embodiments, the expression vector comprises a muscle cell-specific promoter (e.g., Pax7 promoter) operably linked to the polynucleotide sequence encoding the CPEB4 protein.

Related to this aspect of the present invention, a novel use of a CPEB4 enhancer or a compound that increases the expression or activity of CPEB4 protein is further provided for the manufacturing of (1) a medicament for treating age-related diseases; or (2) a kit containing the medicament for treating age-related diseases. In some embodiments, the medicament comprises or consists of an effective amount of the enhancer and one or more physiologically acceptable excipients. In some embodiments, the enhancer comprises an expression vector comprising a polynucleotide sequence encoding a CPEB4 protein and capable of directing the CPEB4 protein expression in targeted cells or tissues, especially muscle cells or muscle tissues.

In a third aspect, the present invention provides a method for assessing the state of senescence in cells or for assessing the state of an age-related condition or disease. The method includes these steps: (a) determining the level of a CPEB4 protein in a first cell; (b) determining the CPEB4 protein level in a second cell of the same kind as the first cell; and (c) determining the first cell as being less senescent than the second cell upon determining the CPEB4 protein level is higher in the first cell than the CPEB4 protein level in the second cell. In some embodiments, the first and second cells are the same cell type: for instance, both are muscle cells or muscle stem cells. In some embodiments, the first and second cells are taken from two anatomic sites of one individual or from two different individuals but preferably of the same cell type or tissue type.

In a forth aspect, this invention provides a method or screening assay useful for identifying compounds that are capable of regulating cellular senescence or aging process and therefore effective for treating age-related diseases or conditions. The method comprises the steps of (i) contacting a cell with a candidate compound; (ii) determining the level of a CPEB4 protein within the cell before and after step (i); and (iii) identifying the compound as an enhancer or promoter of cell senescence upon determining the CPEB4 protein level in step (ii) in the presence of the compound being lower than the CPEB4 protein level in the cell in the absence of the compound prior to step (ii), and identifying the compound as an inhibitor or suppressor of cell senescence upon determining the CPEB4 protein level in step (ii) in the presence of the compound being higher than the CPEB4 protein level in the cell in the absence of the compound. In some embodiments, the cell is a muscle cell or a muscle stem cell. In some embodiments, the cell used in the screening assay endogenously expresses the CPEB4 protein. In some embodiments, the cell used in the assay does not endogenously express the CPEB4 protein but recombinantly expresses the CPEB4 protein.

### BRIEF DESCRIPTION OF THE DRAWINGS

FIG. 1: Geriatric SCs display cellular senescence and mitochondrial defects. a, Schematic illustration showing the mass spectrometry of fixed SCs from PFA-perfused mice (QSCs) at different ages. PFA, paraformaldehyde; FACS, fluorescence-activated cell sorting. b, Principal component analysis (PCA) of the protein expression for adult (3˜6 months), old (18˜24 months) and geriatric (>24 months) SCs. c, Heatmap of representative expression for SC-specific proteins. d, Heatmap of representative expression for autophagy-associated proteins. e, Heatmap of representative expression for Notch and Wnt signaling-related proteins. f, Heatmap of representative expression for senescence-associated proteins. g, Venn diagram of downregulated proteins in geriatric SCs and how many were identified as mitochondrial proteins. h, Reactome ontology analysis of downregulated mitochondrial proteins in geriatric SCs. i, Seahorse analysis of adult and geriatric SCs. FCCP, carbonyl cyanide-p-trifluoromethoxyphenylhydrazone; Rot, rotenone; AA., antimycin a. j, Basal and maximal oxygen consumption rate (OCR) analysis of adult and geriatric SCs. j, *P<0.05, ***P<0.001. n=3. Data shown as mean±s.d.

FIG. 2: CPEB4 is downregulated during aging in various tissues and loss of CPEB4 impairs SC function in adult mice. a, Relative CPEB1-4 relative mRNA levels in various adult and geriatric tissues. *P<0.05, **P<0.01, ***P<0.001. n=3. Data presented as mean±s.d. b, Tibialis anterior (TA) muscles were cryo-sectioned followed by immunostaining for CPEB4 and Pax7. Nuclei were stained with DAPI. c, Quantification of CPEB4 relative fluorescent units (RFUs) in b. *P<0.05, n=3. Data presented as mean±s.d. d, Schematic illustration showing the generation of CPEB4 cKO (conditional knockout) mice. IRES, internal ribosome entry site. STOP, stop cassette. The lower panel shows the experimental design for e and f. e, Analysis of EdU incorporation of wild type (WT) and CPEB4 cKO SCs. SCs were fixed. EdU detection was performed by click chemistry, and the nuclei stained (with DAPI). f, Quantification of EdU⁺ SCs. ****P<0.0001. n=3. Data presented as mean±s.d. g, Schematic illustration showing the workflow of experiments for h-k. h, Cryo-sections of 7 days post-injured (DPI) TA muscles were stained with laminin. Nuclei were stained with DAPI. i, Quantification of fiber size of h. *P<0.05, **P<0.01, ***P<0.001, ****P<0.0001. n=3. Data presented as mean±s.d. j, Cryo-sections of 7 DPI TA muscles were stained for with laminin and Pax7. Nuclei were stained with DAPI. k, Quantification of Pax7+SC number in j. **P<0.01. n=3. Data presented as mean±s.d.

FIG. 3: Loss of CPEB4 induces mitochondrial defects and senescence-associated proteomics landscape. a, Schematic illustration showing the experimental design for mass spectrometry of WT and CPEB4 cKO SCs two weeks after tamoxifen (TMX) injection. b, Correlation heatmap of spearman correlation analysis of protein expression for WT and CPEB4 cKO SCs. c, Heatmap of representative expression for downregulated mitochondrial proteins in CPEB4 cKO SCs. d, Schematic illustration showing the experimental design for mitochondrial respiration analysis of SCs from WT and CPEB4 cKO mice. e, Mitochondrial respiration analysis of WT and CPEB4 cKO SCs. f, Freshly isolated SCs were treated with indicated mitochondrial electron transport chain complex I inhibitor Rotenone or complex III inhibitor Antimycin A for five days followed by SA-β-Gal staining. g, Percentage SA-β-Gal⁺ cells was quantified. *p<0.05, **P<0.01, n=3. Data presented as mean s.d. h, Schematic illustration showing the experimental design for mass spectrometry of WT and CPEB4 cKO SCs three months after TMX injection. i, Correlation heatmap of spearman correlation analysis of protein expression for WT and CPEB4 cKO SCs. j, Functional enrichment analysis of upregulated proteins in CPEB4 cKO SCs in Reactome pathways using g: Profiler. k, Heatmap of representative expression for senescence-related proteins in CPEB4 cKO SCs.

FIG. 4: Restoring CPEB4 expression rejuvenates mitochondrial function and prevents cellular senescence. a, Schematic illustration showing the experimental design for CPEB4 or GFP infection of geriatric SCs. b, Correlation heatmap of spearman correlation analysis of the protein expression for control (GFP-infected) and CPEB4-infected geriatric SCs. c, Venn diagram of upregulated proteins in CPEB4-infected geriatric SCs and mitochondrial proteins (identified from the proteome). d, Heatmap of representative expression for upregulated mitochondrial proteins in CPEB4 overexpressing-geriatric SCs. e, Functional enrichment analysis (Biological Process) of the upregulated mitochondrial proteins in CPEB4 overexpressing geriatric SCs. f, Mitochondrial respiration analysis of GFP and CPEB4-infected SCs. g, Basal and maximal OCR of GFP and CPEB4-infected SCs. h, Mitochondrial ATP production of GFP and CPEB4-infected SCs calculated following the method outlined in Quantifying Cellular ATP Production Rate Using Agilent Seahorse XF Technology. g and h, **P<0.01, ***P<0.001. n=3. Data presented as mean±s.d. i, Equivalent numbers of GFP or CPEB4-infected geriatric SCs were transplanted into 1-day pre-injured geriatric mice for two weeks. TA muscles were sectioned and stained for GFP and laminin. Nuclei were stained with DAPI. j, Quantification of the number of GFP⁺ newly formed muscle fibers. ***P<0.001. n=3. Data presented as mean±s.d. k, Schematic illustration showing the experimental design for CPEB4 infection into human cell lines treated with hydrogen peroxide (H₂O₂). l, SA-β-Gal staining of A172 cell lines after CPEB4 overexpression and H₂O₂ treatment. m, Quantification of SA-β-Gal⁺ cells in 1. n, SA-β-Gal staining of Hela cell lines after CPEB4 overexpression and H₂O₂ treatment. o, Quantification of SA-β-Gal⁺ cells in n. m and o, *p<0.05, **P<0.01, ***P<0.001, ****P<0.0001. n=3. Data presented as mean±s.d.

FIG. 5: SC activation and muscle regeneration are delayed during aging. a-d, In vitro activation analysis of adult and geriatric SCs. a, Immunostaining of Myod1 protein in freshly isolated adult and geriatric SCs. Nuclei were stained with DAPI. b, Quantification of the percentage of Myod1⁺ SCs in a. **P<0.01. n=3. Data presented as mean±s.d. c, EdU incorporation analysis of adult and geriatric SCs. SCs cultured for 30 hours in the presence of EdU were fixed and the EdU detected by click chemistry. Nuclei were stained with DAPI. d, Quantification of EdU⁺ cells. ****P<0.0001. n=3. Data presented as mean±s.d. e-g, Analysis of regenerated fiber size of TA muscles (7 or 14 DPI) from adult and geriatric mice. e, Cryo-sectioned TA muscles were stained for laminin. Nuclei were stained with DAPI. f, Quantification of fiber size of TA muscles (7 DPI). g, Quantification of fiber size in TA muscles (14 DPI). *P<0.05, **P<0.01, ***P<0.001, ****P<0.0001. n=3. Data presented as mean±s.d.

FIG. 6: Geriatric SCs display an impaired transcriptional and translational proteomics landscape. a, Correlation heatmap of spearman correlation analysis of protein expression for adult and geriatric SCs. b, Immunostaining for Pax7 protein in adult and geriatric SCs. c, Quantification of Pax7 RFUs in b. ****P<0.0001. n=3. Data presented as mean±s.d. d, Heatmap of representative expression for spliceosome-related proteins in adult, old and geriatric SCs. e, Heatmap of representative expression for ribosome-related proteins in adult, old and geriatric SCs. f, Functional enrichment analysis of differentially expressed proteins from old SCs versus adult SCs. g, Functional enrichment analysis of differentially expressed proteins from geriatric SCs versus adult SCs. h, Functional enrichment analysis of differentially expressed proteins from geriatric SCs versus old SCs. f-h, Proteins showing significantly higher expression are shown as black bars; proteins with significantly lower expression are shown as white bars (P<0.05).

FIG. 7: Geriatric SCs display defects in the mitochondrial proteome and activity. a, Heatmap of representative expression for oxidative phosphorylation or glycolysis-related proteins in adult, old and geriatric SCs. b, Heatmap of representative expression for downregulated mitochondrial proteins in geriatric SCs. Also shown are three upregulated mitochondrial proteins in geriatric SCs. c, Extracellular acidification rate (ECAR) analysis of adult and geriatric SCs. d, Mitochondrial ATP production from adult and geriatric SCs, *P<0.05. n=3. Data presented as mean±s.d.

FIG. 8: CPEB4 is downregulated in geriatric SCs but loss of CPEB4 did not affect the stem cell pool and muscle fiber size in uninjured muscle. a, Freshly isolated adult or geriatric SCs were stained for CPEB4 and Pax7 protein. Nuclei were stained with DAPI. b, Quantification of CPEB4 RFUs in a. ****P<0.0001, n=3. Data presented as mean s.d. c, Schematic illustration showing the experimental workflow for d-g. d, Cryo-sectioned uninjured TA muscles were stained for laminin and nuclei stained with DAPI. e, Quantification of fiber size in d. n=3. Data are presented as mean±s.d. f, Uninjured TA muscles were stained for laminin and Pax7. Nuclei were stained with DAPI. g, Quantification of the number of Pax7T SCs in f. n=3. Data presented as mean±s.d.

FIG. 9: CPEB4 knockdown delays myogenic lineage progression. a, Freshly prepared single myofibers were stained for Pax7 and CPEB4 protein. Single myofibers cultured for 48 hours were stained for Myod1 and CPEB4. Nuclei were stained with DAPI. b, Quantification of CPEB4 RFUs in a. ****P<0.0001. n=3. Data presented as mean±s.d. c, Primary myoblasts transfected with CPEB4 siRNA for 48 hours were assessed for CPEB4 mRNA using real-time PCR. d, Primary myoblasts transfected with CPEB4 siRNA for 48 hours were assessed for CPEB4 protein expression by Western blot. Beta-tubulin was used as a loading control. ***P<0.001. n=3. Data presented as mean±s.d. e, Analysis of EdU incorporation in negative control (siNC) and CPEB4 knockdown (siCPEB4) SCs on myofibers. Freshly prepared single myofibers were transfected with CPEB4/control siRNA in the presence of EdU. 30 hours post-transfection, fibers were harvested for EdU detection and immunostaining for Myod1 and CPEB4. f, Quantification of EdU⁺ cells that were also Myod1l. ****P<0.0001. n=3. Data presented as mean±s.d. g, Single myofibers were transfected with indicated siRNAs for 36 hours. EdU was added into the culture medium 6 hours before harvesting for EdU detection and Syn4 immunostaining. Nuclei were stained with DAPI. h, Quantification of the number of Syn4⁺ SCs. i, Quantification of the percentage of EdU⁺ SCs. j, Single myofibers were transfected with indicated siRNAs for 72 hours. EdU was added into the culture medium 6 hours before harvesting for EdU detection and Syn4 immunostaining. Nuclei were stained with DAPI. k, Quantification of the number of Syn4⁺ SCs. 1, Quantification of the percentage of EdU⁺ SCs. ***P<0.001, ****P<0.0001. n=3. Data presented as mean±s.d. m, FACS-sorted SCs were transfected with control siRNA/CPEB4 siRNA in the presence of EdU to assess for SC activation. After 36 hours, SCs were examined for EdU incorporation, and nuclei were stained with DAPI. n, Quantification of the percentage of EdU⁺ SCs. o, FACS-sorted SCs were transfected with control siRNA/CPEB4 siRNA for 36 hours. To assess the proliferation rate, 6 hours before harvest EdU was added into the medium. SCs were then examined for EdU incorporation, and nuclei were stained with DAPI. p, Quantification of the percentage of EdU⁺ SCs. **P<0.01. n=3. Data presented as mean±s.d. q, MyoG immunostaining on SCs 72 hours after CPEB4 knockdown. r, Quantification of the percentage of MyoG⁺ SCs. ***P<0.001. n=3. Data presented as mean±s.d.

FIG. 10: Loss of CPEB4 results in defects of the mitochondrial proteome and activity. a, Mass spectrometry of WT and CPEB4 cKO SCs two weeks after TMX injection. The number of differentially expressed proteins in WT and CPEB4 cKO SCs. b, Functional enrichment analysis of upregulated proteins in CPEB4 cKO SCs in Reactome pathways. c, Functional enrichment analysis of downregulated proteins in CPEB4 cKO SCs in Reactome pathways. d, Venn diagram of downregulated proteins in CPEB4 cKO SCs overlapping with mitochondrial proteins. e, Functional enrichment analysis of downregulated mitochondrial proteins in CPEB4 cKO SCs in Reactome pathways. f, Seahorse analysis of SCs from WT control and CPEB4 cKO mice. OCR analysis of SCs from WT control and CPEB4 cKO mice. g, ECAR flux analysis of SCs from WT control and CPEB4 cKO mice. h, Mitochondrial ATP production of SCs from WT control and CPEB4 cKO mice. f-h, **P<0.01, ***P<0.001. n=3. Data presented as mean±s.d.

FIG. 11: CPEB4 knockdown impairs mitochondrial function. a, Primary myoblasts were cultured with FCCP followed by JC-1 FACS-analysis. b, Calculation of the percentage of primary myoblasts in quadrant 2 (Q2). c, SCs were transfected with CPEB4 siRNA. 48 hours after transfection, SCs were harvested for JC-1 FACS-analysis. d, Calculation of the percentage of SCs in Q2. **P<0.01, ***P<0.001, n=3. Data presented as mean±s.d. e, Relative ATP levels after CPEB4 knockdown. **p<0.01, ***P<0.001, n=3. Data presented as mean±s.d.

FIG. 12: CPEB4 binds to mitochondrial protein coding transcripts and interacts with mitochondrial translation machinery. a, Analysis of CPEB4-associated genes using RNA immunoprecipitation sequencing (RIP-seq). Schematic illustration showing the workflow of CPEB4 RIP-seq. b, Volcano plot of genes detected by the two independent CPEB1 RIP experiments. Each dot represents one gene. The X axis represents the −log (adj-P-value), the Y axis represents the log 2 (Fold-change of IP versus IgG). Red dots indicate the genes significantly associated with CPEB4. c, Functional analysis of CPEB4-associated genes in Reactome pathways. d, Functional analysis of CPEB4-associated genes in KEGG pathways.

FIG. 13: CPEB4 interacting proteins correlates with mitochondrial protein translation. a, CPEB4 and Tomm20 immunostaining of 24 hours cultured activated SCs. Cells were imaged by Structured Illumination Microscopy (SIM). b, Schematic illustration showing the workflow of CPEB4 IP followed by mass spectrometry. c, Venn diagram of proteins detected by mass spectrometry after IgG or CPEB4 antibody immunoprecipitation in SCs. The red rectangle shows the proteins targeted by CPEB4. d, Functional enrichment analysis of CPEB4-associated proteins in Cellular Component (CC) pathways. e, The functional protein association networks of CPEB4 specific-associated proteins. f, Reactome pathways analysis of different clusters of these CPEB4-associated proteins.

FIG. 14: Mitochondrial metabolism inhibition induces cellular senescence. a, Primary myoblasts were treated with indicated mitochondrial electron transport chain complex I inhibitor Rotenone or complex III inhibitor Antimycin A for five days, followed by SA-β-Gal staining. b, Quantification of the percentage of SA-β-Gal⁺ cells. *p<0.05, ***P<0.001, n=3. Data presented as mean±s.d.

FIG. 15: Long-term loss of CPEB4 induces severe defects on the mitochondrial proteome. a, Mass spectrometry of WT and CPEB4 cKO SCs three months after TMX injection. The number of differentially expressed proteins in WT and CPEB4 cKO SCs. b, Venn diagram of upregulated proteins in CPEB4 cKO SCs overlapping with mitochondrial proteins. c, Functional enrichment analysis of the downregulated proteins in CPEB4 cKO SCs in Reactome pathways. d, Venn diagram of downregulated proteins in CPEB4 cKO SCs overlapping with mitochondrial proteins. e, Functional enrichment analysis of the downregulated mitochondrial proteins in CPEB4 cKO SCs in Reactome pathways. f, Heatmap of differentially expressed mitochondrial proteins in CPEB4 cKO SCs.

FIG. 16: Restoring CPEB4 expression rejuvenates geriatric cells. a, Mass spectrometry of GFP and CPEB4-infected geriatric SCs. CPEB4 protein expression (raw number of spectra) in infected geriatric SCs. b, Functional enrichment analysis of the upregulated proteins in CPEB4 overexpressing geriatric SCs in Reactome pathways. c, Functional enrichment analysis of the downregulated proteins in CPEB4 overexpressing geriatric SCs in Reactome pathways. d, RNA-sequencing of GFP and CPEB4-infected geriatric SCs. CPEB4 RNA expression (raw read counts) in GFP and CPEB4-infected geriatric SCs. e, Functional enrichment analysis of the upregulated genes in CPEB4 overexpressing geriatric SCs in Reactome pathways. f, Functional enrichment analysis of the downregulated genes in CPEB4 overexpressing geriatric SCs in Reactome pathways. g, Heatmap of representative differentially expressed proteins and RNAs in CPEB4 expressing geriatric SCs. h, Schematic illustration showing the workflow of the CPEB4-infected geriatric SC transplantation experiments. i, An equal number of GFP or CPEB4-infected geriatric SCs were transplanted into 1-day pre-injured geriatric mice for two weeks. TA muscles were sectioned and stained for GFP⁺ Pax7⁺, and laminin. DAPI stained the nuclei. j, Quantification of the number of GFP⁺ Pax7⁺ self-renewed SCs. **P<0.01. n=3. Data presented as mean±s.d. k, Geriatric SCs infected with GFP or CPEB4 containing adenovirus cultured with EdU. 48 hours post-treatment, SCs were fixed for EdU incorporation analysis. Nuclei were stained with DAPI. 1, Quantification of EdU⁺ cells. ***P<0.001. n=3. Data presented as mean±s.d. m, Human cell lines (A172, Daoy, Calu3 and Hela) treated with 50 μM H₂O₂ for two days were infected with GFP or CPEB4 containing adenovirus. 60 hours after infection, cells were cultured with EdU for 12 hours, followed by fixation and EdU incorporation analysis. Nuclei were stained with DAPI. n, Quantification of EdU⁺ cells. *p<0.05, **p<0.01, ***P<0.001.****P>0.0001, n=3. Data presented mean s.d. o, Human cell lines (Daoy and Calu3) treated with 50 μM H₂O₂ for 2 days were infected with GFP or CPEB4 containing adenovirus. Three days after infection, cells were assessed for the presence of SA-β-Gal. p, Quantification of SA-β-Gal⁺ cells. *p<0.05, **p<0.01, ****P<0.0001. n=3. Data presented as mean±s.d.

### DEFINITIONS

The term “CPEB4” refers to cytoplasmic polyadenylation element binding protein 4 or the gene encoding this protein. The human CPEB4 gene is located on human chromosome 5q35.2, and its cDNA sequence is known as NM_030627.4 and set forth as SEQ ID NO:1 in this disclosure. As used herein, the term “CPEB4” encompasses human CPEB4 as well as homologues or orthologues of this protein in other species with at least 80%, 85%, 90%, 95% or higher sequence homology and retaining the same or similar biological activity. The term also includes all variants of the gene product (especially human CPEB4) due to alternative splicing. Exemplary human CPEB4 amino acid sequences are known as NP_085130.2, NP 001295118.1, NP 001295120.1, NP_001295121.1 and NP_001295122.1, set forth herein as SEQ ID NOs:2, 4, 6, 8, and 10.

The term “age-related condition” encompasses any condition, disease, or disorder that is primarily caused by aging and involves gradually diminished normal biological functions of a tissue, organ, or an entire living organism over the passage of time. Several mechanisms of aging have been identified, which include genomic instability, telomere shortening, and cellular senescence. Aging being a primary driving factor, various diseases are now recognized as age-related diseases, including neurodegenerative diseases (dementia, Alzheimer's disease, Parkinson's disease, etc.), inflammatory diseases, cardiovascular diseases, cerebrovascular diseases (e.g., stroke), cancer, arthritis, type 2 diabetes, chronic obstructive pulmonary disease (COPD), hypertension, osteoarthritis/osteoporosis, cataracts, age-related macular degeneration (AMD), hearing loss, immune system disorders, and musculoskeletal disorders.

As used herein, the term “senescence” refers to a biological aging process during which a cell or a living organism exhibits a gradual deterioration or loss of its functional characteristics. Cellular senescence is a state in which cells can no longer divide and thus can no longer support tissue homeostasis, for example, due to a pathological condition. Senescent cells typically manifest a series of defining features: besides being incapable of cell division and having significantly shortened telomere length, senescent cells are often larger than normal cells of the same type and express and secrete certain molecules that normal cells either do not secrete or do so in smaller quantities (e.g., senescence-associated β-galactosidase or SA-β-gal, the tumor suppressor p16INK4a, growth factors, cytokines, and Senescence Associated Secretory Phenotypes (SASPs), among others). The process of reducing or reversing the senescent state or its characteristics of a cell or an organism is referred to as “rejuvenation” (or other grammatically derived versions).

The “muscle cell” refers to a myoblast or myocyte, a specialized animal precursor cell that, when differentiated, can shorten its length by using a series of motor proteins especially arranged in the cell. A muscle cell is a long cell compared to other cell types. A plurality of muscle cells fuse together to create the long muscle fibers present in muscle tissue. Muscle cells are the specialized cells that constitute the three types of muscle tissue of the body: cardiac, skeletal and smooth muscles. “Muscle stem cells” are adult stem cells that reside between skeletal muscle fibers under the basal lamina but outside the basement membrane of the skeletal muscle fiber. Due to their location outside the muscle fiber, muscle stem cells are referred to as “satellite cells,” which are responsible for muscle growth and repair throughout life in response to injury. Muscle stem cells are a heterogeneous population of cells characterized by the expression of the paired-box transcription factor, Pax7, which is essential for the functionality of muscle stem cells. Upon activation, muscle stem cells proliferate and then differentiate to repopulate muscle tissue with new fibers.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

The term “gene” means the segment of DNA involved in producing a polypeptide chain. It may include regions preceding and following the coding region (leader and trailer) as well as intervening sequences (introns) between individual coding segments (exons).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds having a structure that is different from the general chemical structure of an amino acid, but that functions in a manner similar to a naturally occurring amino acid.

There are various known methods in the art that permit the incorporation of an unnatural amino acid derivative or analog into a polypeptide chain in a site-specific manner, see, e.g., WO 02/086075.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, “conservatively modified variants” refers to those nucleic acids that encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V);
6) Phenylalanine (F), Tyrosine (Y), Tryptophan (W);
7) Serine (S), Threonine (T); and
8) Cysteine (C), Methionine (M)
(see, e.g., Creighton, Proteins, W. H. Freeman and Co., N. Y. (1984)).

Amino acids may be referred to herein by either their commonly known three-letter symbols or by the one-letter symbols recommended by the IUPAC-IUB Biochemical Nomenclature Commission. Nucleotides, likewise, may be referred to by their commonly accepted single-letter codes.

In the present application, amino acid residues are numbered according to their relative positions from the left-most residue, which is numbered 1, in an unmodified wild-type polypeptide sequence.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

The term “recombinant” when used with reference, e.g., to a cell, or a nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

A “promoter” is defined as an array of nucleic acid control sequences that direct the transcription of a polynucleotide sequence. As used herein, a promoter includes necessary polynucleotide sequences near the start site of transcription, such as, in the case of a polymerase II type promoter, a TATA element. A promoter also optionally includes distal enhancer or repressor elements, which can be located as many as several thousand base pairs from the start site of transcription. A “constitutive” promoter is a promoter that is active under most environmental and developmental conditions. An “inducible” promoter is a promoter that is only active under environmental or developmental regulation. The term “operably linked” refers to a functional linkage between a polynucleotide expression control sequence (such as a promoter, or array of transcription factor binding sites) and a second polynucleotide sequence, wherein the expression control sequence directs transcription of the polynucleotide sequence corresponding to the second sequence.

An “expression cassette” is a nucleic acid construct, generated recombinantly or synthetically, with a series of specified polynucleotide elements that permit transcription of a particular polynucleotide sequence in a host cell. An expression cassette may be part of a plasmid, viral genome (e.g., a viral vector such as an adenovirus vector or adeno-associated virus vector), or nucleic acid fragment. Typically, an expression cassette includes a polynucleotide to be transcribed, operably linked to a promoter and transcription termination elements.

The term “heterologous” as used in the context of describing the relative location of two elements, refers to the two elements such as polynucleotide sequences (e.g., a promoter or a protein/polypeptide-encoding sequence) or polypeptide sequences (e.g., two peptides as fusion partners within a fusion protein) that are not naturally found in the same relative positions. Thus, a “heterologous promoter” of a gene refers to a promoter that is not naturally operably linked to that gene. Similarly, a “heterologous polypeptide” or “heterologous polynucleotide” to a particular protein or its encoding sequence is one derived from an origin that is different from that particular protein, or if derived from the same origin but not naturally connected to that particular protein or its coding sequence in the same fashion. The fusion of one polypeptide (or its coding sequence) with a heterologous polypeptide (or polynucleotide sequence) does not result in a longer polypeptide or polynucleotide sequence that can be found in nature.

A “host cell” is a cell that contains an expression vector and supports the replication and/or expression of the expression vector. Host cells may be prokaryotic cells such as E. coli, or eukaryotic cells such as yeast, insect, amphibian, or mammalian cells such as CHO, HeLa, muscle cells, and the like, e.g., cultured cells, explants, and cells in vivo.

The term “inhibiting” or “inhibition,” as used herein, refers to any detectable negative effect on a target biological process, such as protein phosphorylation, cellular signal transduction, protein synthesis, cell proliferation, tumorigenicity, and metastatic potential etc. Typically, an inhibition is reflected in a decrease of at least 10%, 20%, 30%, 40%, or 50% in the target process (e.g., the CPEB4 protein level in a muscle cell), or any one of the downstream parameters mentioned above, when compared to a control. Similarly, the term “increasing” or “increase” and the like is used to describe any detectable positive effect on a target biological process, for example, the CPEB4 protein level in a muscle cell, such as a positive change of at least 25%, 50%, 75%, 100%, or as high as 2, 3, 4, 5 or up to 10 or 20 fold, when compared to a control.

The term “effective amount,” as used herein, refers to an amount that is sufficient to produce an intended effect for which a substance is administered. The effect may include a desirable change in a biological process (e.g., increased CPEB4 protein level) as well as the prevention, correction, or inhibition of the progression of symptoms of a disease/condition and related complications to any detectable extent. The exact amount “effective” for achieving the desired effect will depend on the nature of the therapeutic agent, the manner of administration, and the purpose of the treatment and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); and Pickar, Dosage Calculations (1999)).

The term “about” denotes a range of +/−10% of a pre-determined value. For example, “about 10” sets a range of 90% to 110% of 10, i.e., 9 to 11.

### DETAILED DESCRIPTION OF THE INVENTION

### I. Introduction

The proteomics landscape of SCs is altered during aging, showing a senescence proteomics signature and declined functions in maintaining basal stem cell cellular activity with downregulated expression of transcription- and translation-related proteins during aging. Functionally, a subset of mitochondrial proteins is downregulated during aging, with the Seahorse mitochondrial activity assay revealing that mitochondrial metabolism is impaired in SCs. In the studies described herein, the present inventors identified one member of the CPEB protein family, CPEB4, whose levels decline in various aged tissues and functions to regulate the mitochondrial proteomic landscape and activity in SCs. Further analysis revealed that CPEB4 binds to mitochondrial protein coding transcripts and interacts with mitochondrial translation machinery, indicating CPEB4 involvement in the regulation of mitochondrial translation. Moreover, inhibiting mitochondrial activity induces cellular senescence, and long-term loss of CPEB4 results in cellular senescence. Restoring CPEB4 expression rescued impaired mitochondria metabolism in aged cells and prevented a senescence phenotype. As such, methods are provided for the assessment of cellular senescence, rejuvenation of aged cells, and identification of agents capable of modulating senescence.

As a proof-of-concept demonstrating the therapeutic benefits of CPEB4, aged stem cells infected with adenovirus containing the CPEB4 expression sequence led to the rejuvenation of the aged stem cells. Furthermore, aged stem cells re-expressing CPEB4 transplanted into muscles of aged mice demonstrated the ability of CPEB4 to rejuvenate stem cell functions of geriatric SCs following engraftment. The loss of CPEB4 during aging and the restoration of CPEB4 in aged cells presents a diagnostic biomarker and therapeutic target for the treatment of age-related disorders.

### II. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, 10 J. Chrom. 255: 137-149 (1983).

The sequence of a gene of interest, a polynucleotide encoding a polypeptide of interest, and synthetic oligonucleotides can be verified after cloning or subcloning using, e.g., the chain termination method for sequencing double-stranded templates of Wallace et al., Gene 16: 21-26 (1981).

### III. Acquisition of Tissue Samples and Analysis of CPEB4 mRNA

The present invention relates to measuring the amount of CPEB4 mRNA found in a biological tissue sample, such as muscle sample, as a means to assess the level of senescence in the stem cells of these tissues. Thus, the first steps of practicing this invention are to obtain an appropriate tissue sample from a test subject and extract mRNA from the sample.

### A. Acquisition and Preparation of Tissue Samples

A tissue sample is obtained from a person to be tested or monitored for tissue/cell senescence using a method of the present invention. Collection of appropriate tissue samples from an individual is performed in accordance with the standard protocol hospitals or clinics generally follow, such as during a biopsy. An appropriate amount of tissue is collected and may be stored according to standard procedures prior to further processing.

The analysis of CPEB4 mRNA found in a patient's tissue sample according to the present invention may be performed using, e.g., skeletal muscle tissue. The methods for preparing tissue samples for nucleic acid extraction are well known among those of skill in the art. For example, a subject's tissue sample should be first treated to disrupt the cellular membrane so as to release nucleic acids contained within the cells.

### B. Extraction and Quantitation of RNA

There are numerous methods for extracting mRNA from a biological sample. The general methods of mRNA preparation (e.g., described by Sambrook and Russell, Molecular Cloning: A Laboratory Manual 3d ed., 2001) can be followed; various commercially available reagents or kits, such as Trizol reagent (Invitrogen, Carlsbad, CA), Oligotex Direct mRNA Kits (Qiagen, Valencia, CA), RNeasy Mini Kits (Qiagen, Hilden, Germany), and PolyATtract® Series 9600™ (Promega, Madison, WI), may also be used to obtain mRNA from a biological sample from a test subject. Combinations of more than one of these methods may also be used.

It is essential that all contaminating DNA be eliminated from the RNA preparations. Thus, careful handling of the samples, thorough treatment with DNase, and proper negative controls in the amplification and quantification steps should be used.

1. PCR-Based Quantitative Determination of mRNA Level

Once mRNA is extracted from a sample, the amount of human CPEB4 mRNA may be quantified. The preferred method for determining the mRNA level is an amplification-based method, e.g., by polymerase chain reaction (PCR), especially reverse transcription-polymerase chain reaction (RT-PCR).

Prior to the amplification step, a DNA copy (cDNA) of the human CPEB4 mRNA must be synthesized. This is achieved by reverse transcription, which can be carried out as a separate step, or in a homogeneous reverse transcription-polymerase chain reaction (RT-PCR), a modification of the polymerase chain reaction for amplifying RNA. Methods suitable for PCR amplification of ribonucleic acids are described by Romero and Rotbart in Diagnostic Molecular Biology: Principles and Applications pp. 401-406; Persing et al., eds., Mayo Foundation, Rochester, MN, 1993; Egger et al., J. Clin. Microbiol. 33:1442-1447, 1995; and U.S. Pat. No. 5,075,212.

The general methods of PCR are well known in the art and are thus not described in detail herein. For a review of PCR methods, protocols, and principles in designing primers, see, e.g., Innis, et al., PCR Protocols: A Guide to Methods and Applications, Academic Press, Inc. N.Y., 1990. PCR reagents and protocols are also available from commercial vendors, such as Roche Molecular Systems.

PCR is most usually carried out as an automated process with a thermostable enzyme. In this process, the temperature of the reaction mixture is cycled automatically through a denaturing region, a primer annealing region, and an extension reaction region. Machines specifically adapted for this purpose are commercially available.

Although PCR amplification of the target mRNA is typically used in practicing the present invention. One of skill in the art will recognize, however, that amplification of a mRNA species in a sample may be accomplished by any known method, such as ligase chain reaction (LCR), transcription-mediated amplification, and self-sustained sequence replication or nucleic acid sequence-based amplification (NASBA), each of which provides sufficient amplification. More recently developed branched-DNA technology may also be used to quantitatively determine the amount of mRNA species in a sample. For a review of branched-DNA signal amplification for the direct quantitation of nucleic acid sequences in clinical samples, see Nolte, Adv. Clin. Chem. 33:201-235, 1998.

### 2. Other Quantitative Methods

The CPEB4 mRNA can also be detected using other standard techniques, well known to those of skill in the art. Although the detection step is typically preceded by an amplification step, amplification is not required in the methods of the invention. For instance, the mRNA may be identified by size fractionation (e.g., gel electrophoresis), whether or not proceeded by an amplification step. After running a sample in an agarose or polyacrylamide gel and labeling with ethidium bromide according to well-known techniques (see, e.g., Sambrook and Russell, supra), the presence of a band of the same size as the standard comparison is an indication of the presence of a target mRNA, the amount of which may then be compared to the control based on the intensity of the band. Alternatively, oligonucleotide probes specific to CPEB4 mRNA can be used to detect the presence of such mRNA species and indicate the amount of mRNA in comparison to the standard comparison, based on the intensity of signal imparted by the probe.

Sequence-specific probe hybridization is a well-known method of detecting a particular nucleic acid comprising other species of nucleic acids. Under sufficiently stringent hybridization conditions, the probes hybridize specifically only to substantially complementary sequences. The stringency of the hybridization conditions can be relaxed to tolerate varying amounts of sequence mismatch.

A number of hybridization formats well known in the art, including but not limited to, solution phase, solid phase, or mixed phase hybridization assays. The following articles provide an overview of the various hybridization assay formats: Singer et al., Biotechniques 4:230, 1986; Haase et al., Methods in Virology, pp. 189-226, 1984; Wilkinson, In situ Hybridization, Wilkinson ed., IRL Press, Oxford University Press, Oxford; and Hames and Higgins eds., Nucleic Acid Hybridization: A Practical Approach, IRL Press, 1987.

The hybridization complexes are detected according to well-known techniques. Nucleic acid probes capable of specifically hybridizing to a target nucleic acid, i.e., the mRNA or the amplified DNA, can be labeled by any one of several methods typically used to detect the presence of hybridized nucleic acids. One common method of detection is the use of autoradiography using probes labeled with ³H, ¹²⁵I, ³⁵S ¹⁴C, or ³²P, or the like. The choice of radioactive isotope depends on research preferences due to ease of synthesis, stability, and half-lives of the selected isotopes. Other labels include compounds (e.g., biotin and digoxigenin), which bind to anti-ligands or antibodies labeled with fluorophores, chemiluminescent agents, and enzymes. Alternatively, probes can be conjugated directly with labels such as fluorophores, chemiluminescent agents or enzymes. The choice of label depends on the sensitivity required, ease of conjugation with the probe, stability requirements, and available instrumentation.

The probes and primers necessary for practicing the present invention can be synthesized and labeled using well-known techniques. Oligonucleotides used as probes and primers may be chemically synthesized according to the solid phase phosphoramidite triester method first described by Beaucage and Caruthers, Tetrahedron Letts., 22:1859-1862, 1981, using an automated synthesizer, as described in Needham-VanDevanter et al., Nucleic Acids Res. 12:6159-6168, 1984. Purification of oligonucleotides is by either native acrylamide gel electrophoresis or by anion-exchange HPLC as described in Pearson and Regnier, J. Chrom., 255:137-149, 1983.

### IV. Quantitation of Polypeptides

### A. Obtaining Samples

The first step of practicing the present invention is to obtain a sample of the tissue of interest (e.g., skeletal muscles) from a subject being tested, assessed, or monitored for senescence. Samples of the same type should be taken from both a control group (healthy individuals of a predetermined gender and chronological age) and a test group (subjects being tested for assessing their senescence and biological age, for example). Standard procedures routinely employed in hospitals or clinics are typically followed for this purpose, as stated in the previous section.

For the purpose of assessing the biological age and level of senescence in test subjects, individuals' tissue samples may be taken, and the level of human CPEB4 protein may be measured and then compared to a standard control. If a decrease in the level of human CPEB4 protein is observed when compared to the control, the test subject is deemed to have more senescent cells in their tissue sample taken for assessment or a higher biological age compared to the control subjects. For the purpose of monitoring or assessing the levels of senescence in subjects, individual subject's tissue samples may be taken at different time points, such that the levels of human CPEB4 protein can be measured to provide information indicating the state of biological aging and progression of senescence. For instance, when a person's CPEB4 protein (or mRNA) level shows a general trend of a more rapid decrease over time than the rate of decline observed among control subjects, the person is deemed to be aging faster or undergoing senescence faster than his peers of the same chronological age. On the other hand, a lack of substantial change in the rate of decrease in a person's CPEB4 protein (or mRNA) level would indicate a comparable aging process or level of senescence in the patient compared to his peers of the same chronological age. In contrast, when a person's CPEB4 protein (or mRNA) level shows a general trend of a slower rate of decline over time than a rate of decline observed among control subjects, the person is deemed to be aging slower or undergoing senescence at a reduced rate compared to his peers of the same chronological age. Generally, a lower CPEB4 protein (or mRNA) level seen in a patient indicates a more advanced degree of senescence and a more advanced degree of aging. This comparison is meaningful among individuals of similar chronological age.

### B. Preparing Samples for CPEB4 Protein Detection

Tissue samples from a subject suitable for the present invention can be obtained by well-known methods and as described in the previous section. In certain applications of this invention, muscle tissues may be the preferred sample type for CPEB4 protein detection.

### C. Determining the Level of Human CPEB4 Protein

A protein of any particular identity, such as CPEB4 protein, can be detected using a variety of immunological assays. In some embodiments, a sandwich assay can be performed by capturing the polypeptide from a test sample with an antibody having a specific binding affinity for the polypeptide. The polypeptide then can be detected with a labeled antibody having specific binding affinity for the polypeptide. Such immunological assays can be carried out using microfluidic devices such as microarray protein chips. A protein of interest (e.g., human CPEB4 protein) can also be detected by gel electrophoresis (such as 2-dimensional gel electrophoresis) followed by western blot analysis using specific antibodies. Alternatively, standard immunohistochemical techniques can be used to detect a given protein (e.g., human CPEB4 protein) using the appropriate antibodies. Both monoclonal and polyclonal antibodies (including antibody fragments with desired binding specificity) can be used for specific detection of the polypeptide. Such antibodies and their binding fragments with specific binding affinity to a particular protein (e.g., human CPEB4 protein) can be generated by known techniques.

Other methods may also be employed for measuring the level of CPEB4 protein in practicing the present invention. For instance, a variety of methods have been developed based on mass spectrometry technology to rapidly and accurately quantify target proteins in even a large number of samples. These methods involve highly sophisticated equipment such as the triple quadrupole (triple Q) instrument using the multiple reaction monitoring (MRM) technique, matrix assisted laser desorption/ionization time-of-flight tandem mass spectrometer (MALDI TOF/TOF), an ion trap instrument using selective ion monitoring SIM) mode, and the electrospray ionization (ESI) based QTOP mass spectrometer. See, e.g., Pan et al., J Proteome Res. 2009 February; 8(2):787-797.

### V. Establishing a Standard Control

In order to establish a standard control or a comparison basis for practicing the method of this invention, a group of healthy persons free of any known disease (especially any form of disease or disorder relating to inflammation or abnormal metabolism) as conventionally defined is first selected. These individuals are within the appropriate parameters, if applicable, for the purpose of assessing senescence/aging using the methods of the present invention. Optionally, the individuals are of the same gender, similar age (e.g., within +/−5 or 4, 3, or 2 years or 1 year of a pre-determined age), or similar ethnic background to the test subjects.

The healthy status of the selected individuals is confirmed by well-established, routinely employed methods including but not limited to general physical examination of the individuals and general review of their medical history.

Furthermore, the selected group of healthy individuals must be of a reasonable size, such that the average amount/concentration of human CPEB4 mRNA or CPEB4 protein in the tissue sample obtained from the group can be reasonably regarded as representative of the normal or average level among the general population of healthy people of a specific chronological age. Preferably, the selected group comprises at least 10 human subjects.

Once an average value for the CPEB4 mRNA or protein is established based on the individual values found in each subject of the selected healthy control group, this average or median or representative value or profile is considered a standard control. A standard deviation is also determined during the same process. In some cases, separate standard controls may be established for separately defined groups having distinct characteristics such as age, gender, or ethnic background.

### VI. CPEB4 Enhancers and Senescence Inhibitors

By illustrating the correlation of the suppressed expression of CPEB4 protein and increased cellular senescence, the present invention further provides a means for rejuvenating senescent cells and treating patients suffering from aging- or senescence-related conditions: by increasing the level of CPEB4 protein expression or biological activity in cells or relevant tissues. As used herein, treatment of aging- or senescence-related conditions encompasses reducing, reversing, lessening, or eliminating one or more of the symptoms of such conditions, as well as preventing or delaying the onset of one or more of the relevant symptoms. Additionally, since certain risk factors for a hastened aging process (use of tobacco products, excessive consumption of alcohol, unhealthy dietary habits, lack of physical exercise, high-stress living or working environment, etc.) are well known, preventive measures can be prescribed to patients at risk of premature aging such as reducing or eliminating alcohol and tobacco consumption and adopting a healthy diet and active lifestyle. For individuals who have been deemed to have an increased rate of aging by the method of this invention, various treatment strategies are available for treating premature aging in these patients, including but not limited to the administration of an anti-senescence drug, an anti-inflammatory drug, or an agent that effectively increases or enhances the expression level of CPEB4 in the affected cells or tissues (e.g., an enhancer for CPEB4), or any combination thereof.

### A. Increasing CPEB4 Expression

### 1. Nucleic Acids Encoding CPEB4 Proteins

CPEB4 gene expression can be enhanced by using nucleic acids encoding a functional CPEB4 protein. Such nucleic acids can be single-stranded nucleic acids (such as mRNA) or double-stranded nucleic acids (such as DNA) that translate into an active form of CPEB4 protein under favorable conditions.

In one embodiment, the CPEB4-encoding nucleic acid is provided in the form of an expression cassette, typically recombinantly produced, having a promoter operably linked to the polynucleotide sequence encoding the CPEB4 protein. In some cases, the promoter is a universal promoter that directs gene expression in all or most tissue types; in other cases, the promoter is one that directs gene expression specifically in muscle stem cells, for example, a Pax7 promoter. Administration of such nucleic acids can increase CPEB4 protein expression in the target tissue, e.g., muscles especially skeletal muscles. Since the human CPEB4 gene sequence is known as GenBank Accession No. GeneID:80315, and its cDNA sequence is provided herein as SEQ ID NO:1, one can derive a suitable CPEB4-encoding nucleic acid from the sequence, species homologs, and variants of these sequences.

### 2. CPEB4 Proteins

By directly administering an effective amount of an active CPEB4 protein into a senescent cell or to a patient suffering from premature aging and exhibiting depressed CPEB4 protein expression or activity, the cells/tissues or the patient may also be effectively treated and rejuvenated. For example, this can be achieved by delivering/administering a recombinantly produced CPEB4 protein possessing its biological activity to the affected cells/tissues or the patient. Formulations and methods for delivering a protein- or polypeptide-based therapeutic agent are well known in the art.

### 3. Activators of CPEB4 Protein

Increased CPEB4 protein activity can be achieved with an agent that is capable of activating the expression of CPEB4 protein or enhancing the activity of CPEB4 protein. For example, an activating agent may be able to activate CPEB4 gene expression by activating the promoter region of this gene. Other activating agents may include transcriptional activators specific for the CPEB4 promoter and/or enhancer. Such activating agents can be screened for and identified using the CPEB4 expression assays described in the examples herein.

Conversely, agonists of CPEB4 expression either at mRNA or protein level are another type of activators of CPEB4 expression. Activators of this nature, such as an activating antibody, may act by enhancing the biological activity of the CPEB4 protein, typically (but not necessarily) by direct binding with the CPEB1 protein and/or its interacting proteins. Preliminary screening for such agonists may start with a binding assay for identifying molecules that physically interact with CPEB4 protein.

### B. Identifying Additional CPEB4 Enhancers

Compounds capable of modulating (especially enhancing) CPEB4 expression can be of virtually any chemical and structural nature: they may be macromolecules such as polypeptides, polynucleotides, lipids, or small molecules including inorganic compounds. As long as they possess a confirmed positive effect on promoting CPEB4 expression, such enhancers may be useful for promoting CPEB4 expression and therefore useful for reducing or reversing cellular senescence as well as for treating age-related conditions and disorders.

The modulators of CPEB4 are useful for their capacity to regulate the senescent state of cells, acting as anti-aging therapeutics for patients suffering from diseases or conditions involving loss of biological functions due to aging. Assays for confirming such regulatory effects of a modulator can be performed in vitro or in vivo. An in vitro assay typically involves exposure of cultured cells (such as muscle cells) to a candidate compound and monitoring subsequent changes in the level or activity of CPEB4 in the cells. For example, following exposure to an inhibitor or enhancer of CPEB4 at an adequate concentration for an appropriate duration of time, suitable cells (such as muscle cells or muscle stem cells) are examined for any potential changes in their CPEB4 synthesis rate by immunoassays such as Western blot and in situ immunostaining, etc. A positive change or increase of CPEB4 expression level due to exposure to a compound in comparison to the CPEB4 protein level in the cells in the absence of the compound indicates the compound's role as an enhancer of CPEB4; conversely, a negative change or decrease indicates the compound's role as an inhibitor of CPEB4. For example, an inhibitory effect is detected when a decrease in CPEB4 level by at least 10%, 20%, 30%, 40%, 50%, 60%, 70%, 80%, 90% or more is observed, whereas an enhancing effect is detected when an increase in CPEB4 level by at least 50%, 80%, 100%, or by at least 2 fold, 3-fold, 5-fold, 10-fold, 20-fold or more is observed.

The enhancing or inhibitory effects on CPEB4 expression by a CPEB4 enhancer or inhibitor can also be demonstrated using in vivo assays. For example, a compound proposed as a CPEB4 inhibitor or enhancer can be delivered (e.g., by injection) into an animal, and the cellular levels of CPEB4 before and after administration of the compound can be assessed. Injection methods can be subcutaneous, intramuscular, intravenous, or intraperitoneal in nature. Changes in CPEB4 levels and other features indicative of cellular senescence can be subsequently monitored by various means, such as measuring the pertinent biological functions of treated animals with a control group of animals having similar chronological age/physical condition but not given the compound. The Examples section of this disclosure provides detailed descriptions of some exemplary in vivo assays. An enhancing effect is detected when a reduction of senescence or increase in preservation/regaining of biological functions is established in the test group. Preferably, the positive effect is at least a 10% increase, more preferably, the increase is at least 20%, 30%, 40%, 50%, 60%, 70%, 80%, 90%, 100%, 200%, 500%, or higher in the preservation of function.

As stated above, CPEB4 modulators can have diverse chemical and structural features. For instance, an inhibitor can be a non-functional CPEB4 mutant that retains the binding ability of CPEB4 to its upstream or downstream effector molecules, a neutralizing antibody to CPEB4 that interferes with CPEB4-mediated activity, or any small molecule or macromolecule that simply hinders the interaction between CPEB4 and its upstream or downstream effector molecules. Conversely, an enhancer can be a CPEB4 variant with increased binding ability to CPEB4's upstream or downstream effector molecules, or any small molecule or macromolecule that promotes the interaction between CPEB4 and its upstream or downstream effector molecules. Essentially any chemical compound can be tested as a potential inhibitor or enhancer of CPEB4. Most preferred are generally compounds that can be dissolved in aqueous or organic (especially DMSO-based) solutions. Inhibitors can be identified by screening a combinatorial library containing many potentially effective compounds. Such combinatorial chemical libraries can be screened in one or more assays, as described herein, to identify those library members (particular chemical species or subclasses) that display a desired characteristic activity. The compounds thus identified can serve as conventional “lead compounds” or can themselves be used as potential or actual therapeutics.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)) and carbohydrate libraries (see, e.g., Liang et al., Science, 274:1520-1522 (1996) and U.S. Pat. No. 5,593,853). Other chemistries for generating chemical diversity libraries can also be used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with β-D-glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see, Ausubel, Berger and Sambrook, allsupra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and benzodiazepines, U.S. Pat. No. 5,288,514).

### V. Pharmaceutical Compositions and Administration

The present invention also provides pharmaceutical compositions or physiological compositions comprising an effective amount of a compound that enhances CPEB4 expression, such as a nucleic acid encoding an active CPEB4 protein, small chemicals, peptides, proteins, macromolecules, or the like, in both prophylactic and therapeutic applications for rejuvenating aging cells, tissues, or organisms. Such pharmaceutical or physiological compositions also include one or more pharmaceutically or physiologically acceptable excipients or carriers. Pharmaceutical compositions of the invention are suitable for use in a variety of drug delivery systems. Suitable formulations for use in the present invention are found in Remington's Pharmaceutical Sciences, Mack Publishing Company, Philadelphia, PA, 17th ed. (1985). For a brief review of methods for drug delivery, see, e.g., Langer, Science 249: 1527-1533 (1990).

The pharmaceutical compositions of the present invention can be administered by various routes, e.g., oral, subcutaneous, transdermal, intramuscular, intravenous, or intraperitoneal. The preferred routes of administering the pharmaceutical compositions are local delivery to a relevant organ or tissue in a patient suffering from a condition involving premature aging at daily doses of about 0.01-2500 mg, preferably 2.5-500 mg or 10-100 mg, of a CPEB4 enhancer for a 70 kg adult human per day. The appropriate dose may be administered in a single daily dose or as divided doses presented at appropriate intervals, for example as two, three, four, or more subdoses per day.

For preparing pharmaceutical compositions containing a CPEB4 enhancer and one or more pharmaceutically acceptable carriers are used. The pharmaceutical carrier can be either solid or liquid. Solid form preparations include, for example, powders, tablets, dispersible granules, capsules, cachets, and suppositories. A solid carrier can be one or more substances that can also act as diluents, flavoring agents, solubilizers, lubricants, suspending agents, binders, or tablet disintegrating agents; it can also be an encapsulating material.

In powders, the carrier is generally a finely divided solid that is in a mixture with the finely divided active component, e.g., midostaurin, axitinib, bosutinib, or ruxolitinib. In tablets, the active ingredient (e.g., an enhancer of CPEB4) is mixed with the carrier having the necessary binding properties in suitable proportions and compacted in the shape and size desired.

For preparing pharmaceutical compositions in the form of suppositories, a low-melting wax such as a mixture of fatty acid glycerides and cocoa butter is first melted and the active ingredient is dispersed therein by, for example, stirring. The molten homogeneous mixture is then poured into convenient-sized molds and allowed to cool and solidify.

Powders and tablets preferably contain between about 5% to about 70% by weight of the active ingredient of an enhancer of CPEB4. Suitable carriers include, for example, magnesium carbonate, magnesium stearate, talc, lactose, sugar, pectin, dextrin, starch, tragacanth, methyl cellulose, sodium carboxymethyl cellulose, a low-melting wax, cocoa butter, and the like.

The pharmaceutical compositions can include the formulation of the active compound of a CPEB4 enhancer with encapsulating material as a carrier providing a capsule in which the inhibitor (with or without other carriers) is surrounded by the carrier, such that the carrier is thus in association with the compound. In a similar manner, cachets can also be included. Tablets, powders, cachets, and capsules can be used as solid dosage forms suitable for oral administration.

Liquid pharmaceutical compositions include, for example, solutions suitable for oral or parenteral administration, suspensions, and emulsions suitable for oral administration. Sterile water solutions of the active component (e.g., a CPEB4 enhancer) or sterile solutions of the active component in solvents comprising water, buffered water, saline, PBS, ethanol, or propylene glycol are examples of liquid compositions suitable for parenteral administration. The compositions may contain pharmaceutically acceptable auxiliary substances as required to approximate physiological conditions, such as pH adjusting and buffering agents, tonicity adjusting agents, wetting agents, detergents, and the like.

Sterile solutions can be prepared by dissolving the active component (e.g., a CPEB4 enhancer) in the desired solvent system, and then passing the resulting solution through a membrane filter to sterilize it or, alternatively, by dissolving the sterile compound in a previously sterilized solvent under sterile conditions. The resulting aqueous solutions may be packaged for use as is, or lyophilized, the lyophilized preparation being combined with a sterile aqueous carrier prior to administration. The pH of the preparations typically will be between 3 and 11, more preferably from 5 to 9, and most preferably from 7 to 8.

The pharmaceutical compositions containing a CPEB4 enhancer can be administered for prophylactic and/or therapeutic treatments. In therapeutic applications, compositions are administered to a patient already suffering from a condition that involves or is exacerbated by the aging process in an amount sufficient to prevent, cure, reverse, or at least partially slow or arrest the symptoms of the condition and its complications, such as the onset, progression, duration, and severity of the disease. An amount adequate to accomplish this is defined as a “therapeutically effective dose.” Amounts effective for this use will depend on the severity of the disease or condition and the weight and general state of the patient, but generally range from about 0.1 mg to about 2,500 mg of the CPEB4 enhancer per day for a 70 kg patient, with dosages of from about 2.5 mg to about 500 mg of the CPEB4 enhancer per day for a 70 kg patient being more commonly used.

In prophylactic applications, pharmaceutical compositions containing a CPEB4 enhancer are administered to a patient susceptible to or otherwise at risk of developing a disease involving or exacerbated by the aging process, in an amount sufficient to delay or prevent the onset of the symptoms. Such an amount is defined to be a “prophylactically effective dose.” In this use, the precise amounts of the CPEB4 enhancer again depend on the patient's state of health and weight, but generally range from about 0.1 mg to about 2,500 mg of the CPEB4 enhancer for a 70 kg patient per day, more commonly from about 2.5 mg to about 500 mg for a 70 kg patient per day.

Single or multiple administrations of the compositions can be carried out with dose levels and pattern being selected by the treating physician. In any event, the pharmaceutical formulations should provide a quantity of a CPEB4 enhancer sufficient to effectively promote CPEB4 expression in the patient, either therapeutically or prophylactically.

### VI. Therapeutic Applications Using Nucleic Acids

A variety of conditions can be treated by therapeutic approaches that involve introducing a nucleic acid encoding a polypeptide enhancer of CPEB4 into a cell such that the coding sequence is transcribed and the polypeptide enhancer is produced in the cell. For discussions on the application of gene therapy towards the treatment of genetic as well as acquired diseases, see, Miller Nature 357:455-460 (1992); and Mulligan Science 260:926-932 (1993).

### A. Vectors for Gene Delivery

For delivery to a cell or organism, a polynucleotide encoding a polypeptide that enhances CPEB4 expression (including an active form or CPEB4 protein itself) can be incorporated into a vector. Examples of vectors used for such purposes include expression plasmids capable of directing the expression of the nucleic acids in the target cell. In other instances, the vector is a viral vector system wherein the polynucleotide is incorporated into a viral genome. The virus carrying this polynucleotide is capable of transfecting the target cell. In one embodiment, the encoding polynucleotide can be operably linked to expression and control sequences that can direct the expression of the polypeptide or oligonucleotide in the desired target cells. Thus, one can achieve expression of the polypeptide enhancer under appropriate conditions in the target cell; for example, a muscle stem cell-specific promoter (e.g., Pax7 promoter) may be used in an expression vector to direct the expression of CPEB4 protein in satellite cells.

### B. Gene Delivery Systems

Viral vector systems useful in the expression of a polypeptide enhancer of CPEB4 include, for example, naturally occurring or recombinant viral vector systems. Depending on the particular application, suitable viral vectors include replication-competent, replication-deficient, and conditionally replicating viral vectors. For example, viral vectors can be derived from the genome of human or bovine adenoviruses, vaccinia virus, herpes virus, adeno-associated virus, minute virus of mice (MVM), HIV, sindbis virus, and retroviruses (including but not limited to Rous sarcoma virus and lentivirus), and MoMLV. Typically, the coding sequence of interest (e.g., one encoding for a polypeptide CPEB4 enhancer of the present invention) is inserted into such vectors to allow packaging of the gene construct, typically with accompanying viral DNA, followed by infection of a targeted host cell and expression of the coding sequence of interest (e.g., one encoding for an active form of CPEB4 protein).

As used herein, “gene delivery system” refers to any means for the delivery of a polynucleotide sequence of the invention to a target cell. In some embodiments of the invention, nucleic acids are conjugated to a cell receptor ligand for facilitated uptake (e.g., invagination of coated pits and internalization of the endosome) through an appropriate linking moiety, such as a DNA linking moiety (Wu et al., J. Biol. Chem. 263:14621-14624 (1988); WO 92/06180), or by ultrasound-microbubble delivery system (Lan H Y et al., J. Am Soc. Nephrol. 14:1535-1548). For example, nucleic acids may comprise a Pax7 promoter, which is a promoter to drive protein expression specifically in muscle stem cells.

Similarly, viral envelopes used for packaging gene constructs that include the nucleic acids of the invention can be modified by the addition of receptor ligands or antibodies specific for a receptor to permit receptor-mediated endocytosis into specific cells (see, e.g., WO 93/20221, WO 93/14188, and WO 94/06923). In some embodiments of the invention, the DNA constructs of the invention are linked to viral proteins, such as adenovirus particles, to facilitate endocytosis (Curiel et al., Proc. Natl. Acad. Sci. U.S.A. 88:8850-8854 (1991)). In other embodiments, the inhibitors of the instant invention can include microtubule inhibitors (WO/9406922), synthetic peptides mimicking influenza virus hemagglutinin (Plank et al., J. Biol. Chem. 269:12918-12924 (1994)), and nuclear localization signals such as SV40 T antigen (WO93/19768).

Retroviral vectors may also be useful for introducing the coding sequence of a polypeptide CPEB4 enhancer of the invention into target cells, tissues, or organisms (e.g., muscles, heart, liver, epithelium, and gastrointestinal tract, especially small intestine). Retroviral vectors are produced by genetically manipulating retroviruses. The viral genome of retroviruses is RNA. Upon infection, this genomic RNA is reverse transcribed into a DNA copy which is integrated into the chromosomal DNA of transduced cells with a high degree of stability and efficiency. The integrated DNA copy is referred to as a provirus and is inherited by daughter cells as is any other gene. The wild-type retroviral genome and the proviral DNA have three genes: the gag, the pol and the env genes, which are flanked by two long terminal repeat (LTR) sequences. The gag gene encodes the internal structural (nucleocapsid) proteins; the pol gene encodes the RNA directed DNA polymerase (reverse transcriptase); and the env gene encodes viral envelope glycoproteins. The 5′ and 3′ LTRs serve to promote transcription and polyadenylation of virion RNAs. Adjacent to the 5′ LTR are sequences necessary for reverse transcription of the genome (the tRNA primer binding site) and for efficient encapsulation of viral RNA into particles (the Psi site) (see, Mulligan, In: Experimental Manipulation of Gene Expression, Inouye (ed), 155-173 (1983); Mann et al., Cell 33:153-159 (1983); Cone and Mulligan, Proceedings of the National Academy of Sciences, U.S.A., 81:6349-6353 (1984)).

The design of retroviral vectors is well-known to those of ordinary skill in the art. In brief, if the sequences necessary for encapsulation (or packaging of retroviral RNA into infectious virions) are missing from the viral genome, the result is a cis acting defect which prevents encapsulation of genomic RNA. However, the resulting mutant is still capable of directing the synthesis of all virion proteins. Retroviral genomes from which these sequences have been deleted, as well as cell lines containing the mutant genome stably integrated into the chromosome are well known in the art and are used to construct retroviral vectors. Preparation of retroviral vectors and their uses are described in many publications including, e.g., European Patent Application EPA 0 178 220; U.S. Pat. No. 4,405,712; Gilboa Biotechniques 4:504-512 (1986); Mann et al., Cell 33:153-159 (1983); Cone and Mulligan Proc. Natl. Acad. Sci. USA 81:6349-6353 (1984); Eglitis et al. Biotechniques 6:608-614 (1988); Miller et al. Biotechniques 7:981-990 (1989); Miller (1992) supra; Mulligan (1993), supra; and WO 92/07943.

The retroviral vector particles are prepared by recombinantly inserting the desired nucleotide sequence into a retrovirus vector and packaging the vector with retroviral capsid proteins by use of a packaging cell line. The resultant retroviral vector particle is incapable of replication in the host cell but is capable of integrating into the host cell genome as a proviral sequence containing the desired nucleotide sequence. As a result, the patient is capable of producing, for example, a polypeptide CPEB4 enhancer of the invention and thus this may restore the target cells (e.g., muscle cells) back to a normal phenotype, for example, rejuvenated cells or tissues, with reduced signs of senescence, with preserved or restoration of biological functions.

Packaging cell lines that are used to produce the retroviral vector particles are typically mammalian tissue culture cell lines, which are capable of high levels of protein expression. The defective retroviral vectors used, on the other hand, lack these structural genes but encode the remaining proteins necessary for packaging. To prepare a packaging cell line, one can construct an infectious clone of a desired retrovirus in which the packaging site has been deleted. Cells comprising this construct will express all structural viral proteins, but the introduced DNA will be incapable of being packaged. Alternatively, packaging cell lines can be produced by transforming a cell line with one or more expression plasmids encoding the appropriate core and envelope proteins. In these cells, the gag, pol, and env genes can be derived from the same or different retroviruses.

A number of packaging cell lines suitable for the present invention are also available in the prior art. Examples of these cell lines include Crip, GPE86, PA317 and PG13 (see Miller et al., J. Virol. 65:2220-2224 (1991)). Examples of other packaging cell lines are described in Cone and Mulligan Proceedings of the National Academy of Sciences, USA, 81:6349-6353 (1984); Danos and Mulligan Proceedings of the National Academy of Sciences, USA, 85:6460-6464 (1988); Eglitis et al. (1988), supra; and Miller (1990), supra.

Packaging cell lines capable of producing retroviral vector particles with chimeric envelope proteins may be used. Alternatively, amphotropic or xenotropic envelope proteins, such as those produced by PA317 and GPX packaging cell lines may be used to package the retroviral vectors.

### C. Pharmaceutical Formulations

When used for pharmaceutical purposes, the nucleic acid encoding a polypeptide CPEB4 enhancer is generally formulated in a suitable buffer, which can be any pharmaceutically acceptable buffer, such as phosphate-buffered saline or sodium phosphate/sodium sulfate, Tris buffer, glycine buffer, sterile water, and other buffers known to the ordinarily skilled artisan such as those described by Good et al. Biochemistry 5:467 (1966).

The compositions can additionally include a stabilizer, enhancer or other pharmaceutically acceptable carriers or vehicles. A pharmaceutically acceptable carrier can contain a physiologically acceptable compound that acts, for example, to stabilize the nucleic acids of the invention and any associated vector. A physiologically acceptable compound can include, for example, carbohydrates, such as glucose, sucrose or dextrans, antioxidants, such as ascorbic acid or glutathione, chelating agents, low molecular weight proteins or other stabilizers or excipients. Other physiologically acceptable compounds include wetting agents, emulsifying agents, dispersing agents, or preservatives, which are particularly useful for preventing the growth of microorganisms. Various preservatives are well known and include, for example, phenol and ascorbic acid. Examples of carriers, stabilizers or adjuvants can be found in Remington's Pharmaceutical Sciences, Mack Publishing Company, Philadelphia, PA, 17th ed. (1985).

### D. Administration of Formulations

The formulations containing a polynucleotide sequence encoding a polypeptide CPEB4 enhancer can be delivered to the target tissue or organ using any delivery method known to the ordinarily skilled artisan. In some embodiments of the invention, the encoding polynucleotide sequences are formulated for subcutaneous, intramuscular, intravenous, or intraperitoneal injection, or for oral ingestion, or for topical application.

The formulations containing the nucleic acid encoding an enhancer of the invention are typically administered to a cell. The cell can be present as part of a tissue, such as muscle cells or muscle stem cells as a part of the skeletal muscle system, or as an isolated cell, such as in tissue culture. The cell can be provided in vivo, ex vivo, or in vitro.

The formulations can be introduced into the tissue of interest in vivo or ex vivo by a variety of methods. In some embodiments of the invention, the nucleic acids of the invention are introduced into cells by such methods as microinjection, calcium phosphate precipitation, liposome fusion, ultrasound, electroporation, or biolistics. In further embodiments, the nucleic acids are taken up directly by the tissue of interest, for example, when the targeted cells are the red blood cells intravenous injection is appropriate.

In some embodiments of the invention, the nucleic acids of the invention are administered ex vivo to cells or tissues explanted from a patient, then returned to the patient. Examples of ex vivo administration of therapeutic gene constructs include Nolta et al., Proc Natl. Acad. Sci. USA 93(6):2414-9 (1996); Koc et al., Seminars in Oncology 23(1):46-65 (1996); Raper et al., Annals of Surgery 223(2):116-26 (1996); Dalesandro et al., J. Thorac. Cardi. Surg., 11(2):416-22 (1996); and Makarov et al., Proc. Natl. Acad. Sci. USA 93(1):402-6(1996).

The effective dosage of the formulations will vary depending on many different factors, including means of administration, target site, physiological state of the patient, and other medicines administered. Thus, treatment dosages will need to be titrated to optimize safety and efficacy. In determining the effective amount of the vector to be administered, the physician should evaluate the particular nucleic acid used, the disease state of the patient; the age, weight, and overall condition of the patient circulating plasma levels, vector toxicities, progression of the disease, and the production of anti-vector antibodies. The dosage also will be determined by the existence, nature, and extent of any adverse side effects that accompany the administration of a particular vector. To practice the present invention, doses of CPEB4 enhancers ranging from about 0.1 μg-100 mg per patient are typical. Doses generally range between about 0.01 and about 100 μg per kilogram of body weight, preferably between about 0.1 and about 50 μg/kg of body weight or about 10⁸-10¹⁰ or 10¹² particles per injection. In general, the dose equivalent of a naked nucleic acid from a vector is from about 1 μg-100 μg for a typical 70 kg patient, and doses of vectors that include a retroviral particle are calculated to yield an equivalent amount of nucleic acid encoding a polypeptide that enhances CPEB4 expression.

### VII. Kits

The invention also provides kits for reversing cellular senescence and therefore for treating age-related conditions according to the method of the present invention. The kits typically include a container that contains (1) a pharmaceutical composition having an effective amount of an enhancer of CPEB4 (for instance, an active form of CPEB4 protein or an expression vector encoding an active form of CPEB4 protein) and (2) informational material containing instructions on how to dispense the pharmaceutical composition, including a description of the type of patients who may be treated (e.g., human patients suffering from one or more age-related diseases or conditions), the schedule (e.g., dose and frequency) and route of administration, and the like. In some cases, a second container is included in the kit to provide a second pharmaceutical composition comprising an effective amount of a second anti-aging therapeutic agent.

### EXAMPLES

The following examples are provided by way of illustration only and not by way of limitation. Those of skill in the art will readily recognize a variety of non-critical parameters that could be changed or modified to yield essentially similar results.

### ABSTRACT

Age-associated impairments in stem cell function correlate with a decline in somatic tissue regeneration capacity following tissue injury or during disease. Cellular metabolism is altered during the aging process and therefore interventions to improve metabolic defects can be utilized to rejuvenate aged cells. The compositions and methods are herein provided for illustrating the global alterations of the proteomic landscape during aging; determination of defects in mitochondrial metabolism in aged cells; diagnosis of aging-related disorders based on the decreased levels of CPEB4 in various aged tissues; and rejuvenation of aged cells by restoring CPEB4 expression to reverse senescence.

### INTRODUCTION

### Mitochondrial Function and Stem Cell Aging

Mitochondrial activity is important in the regulation of stem cell fate and function, whether it be in healthy tissue or during aging and disease (Ansó et al., 2017; Bhattacharya and Scimè, 2020; Khacho et al., 2016; Lisowski et al., 2018; Mohrin et al., 2015; Papa et al., 2019). Mitochondria are the major organelle for energy harvesting but also have a signaling function, being a source of stress-induced retrograde signals, such as reactive oxygen species (ROS), which can influence other cellular sites and are known to affect stem cell function (Butow and Avadhani, 2004; Chandel, 2014; Guha et al., 2014; Li et al., 2017). The ROS accumulates in SCs during aging, with its depletion leading to the rejuvenation of geriatric SCs (García-Prat et al., 2016). Mitochondria also compartmentalize several key metabolic pathways, such as the tricarboxylic acid (TCA) cycle, fatty acid β-oxidation, from which the metabolites also function as retrograde signals (Chandel et al., 2016; Martinez-Reyes et al., 2016; Ryall et al., 2015; Teperino et al., 2010; Zhang et al., 2016). As mitochondria play an important role in regulating stem cell activity, the age-dependent decline in mitochondrial function has been reported in various tissues (Ahlqvist et al., 2015; Chistiakov et al., 2014; Cho et al., 2011; Haas, 2019; Payne and Chinnery, 2015; Stoll et al., 2011; Sun et al., 2016). Herein, we revealed the mitochondria proteomic landscape of SCs is altered during the SC aging process. Moreover, the mitochondrial activity in terms of OCR and ATP production declines dramatically in geriatric SCs. Collectively, others and our work provide the evidence to potentially treat age-related diseases or reverse aging by modulating mitochondrial function or the transplantation of young mitochondria into other tissues or other stem cells.

### CPEB4 Function and Mitochondrial Metabolism Regulation

CPEB4 regulates specific cellular functions by targeting corresponding transcripts (Calderone et al., 2016; Cao et al., 2018; Lu et al., 2017a; Parras et al., 2018). It is well established that the CPEB protein family regulates translation through cytoplasmic polyadenylation of bound transcripts (Fernández-Miranda and Méndez, 2012; Richter, 2007). For example, CPEB4 collaborates with CPEB1 to regulate tumor progression and mitosis by targeting transcripts encoding cell cycle-related proteins (Novoa et al., 2010). Besides, CPEB4 is involved in neuronal disease or neurodevelopment (Huang et al., 2006; Shin et al., 2016) with a mutation of CPEB4 correlating with autism-like disease (Parras et al., 2018). Furthermore, CPEB4 induces glycolysis and activates hepatic stellate cells to promote liver fibrosis by increasing the expression of PFKFB3 (Mejias et al., 2020). Besides, CPEB4 regulates mitochondrial fatty acid oxidation and endoplasmic reticulum (ER)-related proteins expression to prevent lipid accumulation in the liver during aging (Maillo et al., 2017).

Herein, we reveal that CPEB4 is involved in the regulation of the mitochondrial proteome and activity. Mechanistically, we identified whole transcriptomic targets of CPEB4 in SCs and found that many CPEB4-bound transcripts are mitochondrial protein-encoding transcripts. CPEB4-associated genes are mainly enriched in mitochondrial metabolism-related pathways. Moreover, our super-resolution SIM images of CPEB4 and Tomm20 coimmunostaining showed that part of the CPEB4 protein is located inside mitochondria. Besides, our CPEB4 antibody IP-mass spectrometry experiments identified the interacting partner of CPEB4. Surprisingly, CPEB4 interacts with mitochondrial translational machinery. These observations provide a new angle on how CPEB4 regulates translation by directly binding to translation machinery independently of cytoplasmic polyadenylation. The detailed mechanisms warrant further studies. In addition, the translation location of mitochondrial proteins encoding transcripts is a topic of great debate. Other than translation in cytoplasmic ribosomes, mitochondrial protein-encoding mRNAs were shown to be transported into the mitochondria for translation through mitochondrial translation machinery (D'Souza and Minczuk, 2018; Eliyahu et al., 2010; Lesnik et al., 2015; Popow et al., 2015). Our data here raises the possibility that the RNA-binding protein CPEB4 may function as a transporter of mitochondrial protein-encoding transcripts for their targeting to mitochondrial translation machinery.

### CPEB4 and Stem Cell Aging

CPEB4 is essential for tissue homeostasis during aging. For example, CPEB4 is required for adaptation to high-fat diet-induced and aging-induced ER stress, and subsequent hepatosteatosis (Maillo et al., 2017). Herein, our proteomics landscape analysis of CPEB4 cKO SCs revealed that loss of CPEB4 induced the expression of senescence-related proteins. Re-expression of CPEB4 in geriatric SCs improved the function of geriatric SCs, including mitochondrial metabolism, SC activation, muscle regeneration, and self-renewal. These data indicate that CPEB4 is a key player to prevent stem cell aging and provides a link between mitochondria activity and stem cell aging. Besides, CPEB4 deletion resulted in the accumulation of cellular ROS generated from mitochondria (Hu et al., 2018). The depletion of ROS rejuvenated geriatric SCs (García-Prat et al., 2016). We observed that upregulated proteins in geriatric SCs and CPEB4 cKO SCs are enriched in “Oxidative stress induced senescence” pathways. Moreover, restoring CPEB4 expression rejuvenates geriatric SCs. Thus, CPEB4 also provides a potential mechanism to regulate SC aging by preventing ROS accumulation.

### Results

### Example 1. Geriatric SCs Display Cellular Senescence

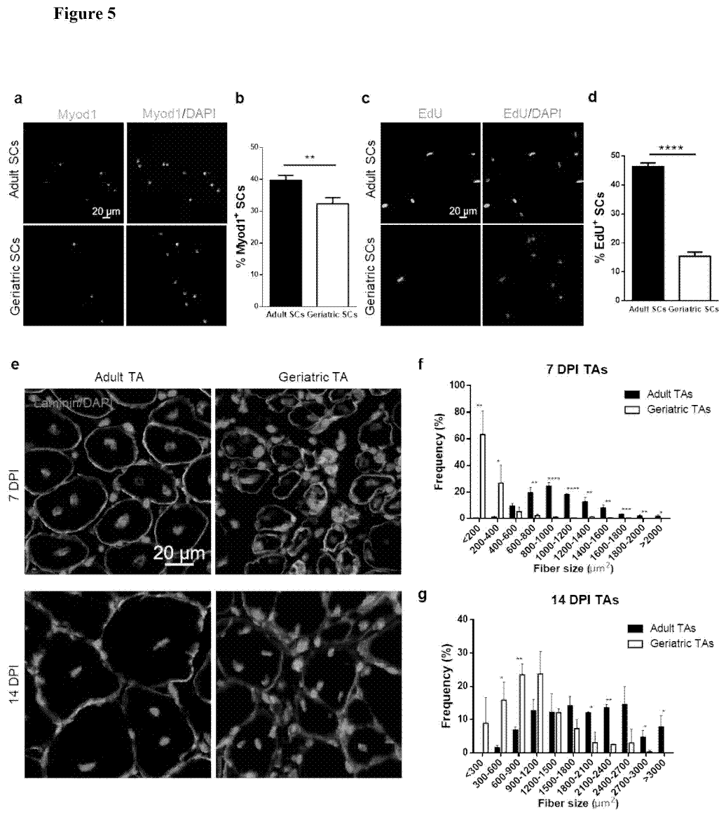

Consistent with previous studies (García-Prat et al., 2016; Sousa-Victor et al., 2014), we found that SCs from very old mice (>24 months, geriatric mice) have reduced capability for activation and muscle regeneration. The percentage of Myod1⁺ SCs or EdU⁺ SCs was decreased when compared to adult SCs (isolated from 3˜6 months adult mice) (FIGS. 5a-d). The size of newly regenerated myofibers (centrally nucleated) was dramatically reduced in geriatric mice after injury (FIGS. 5e-g).

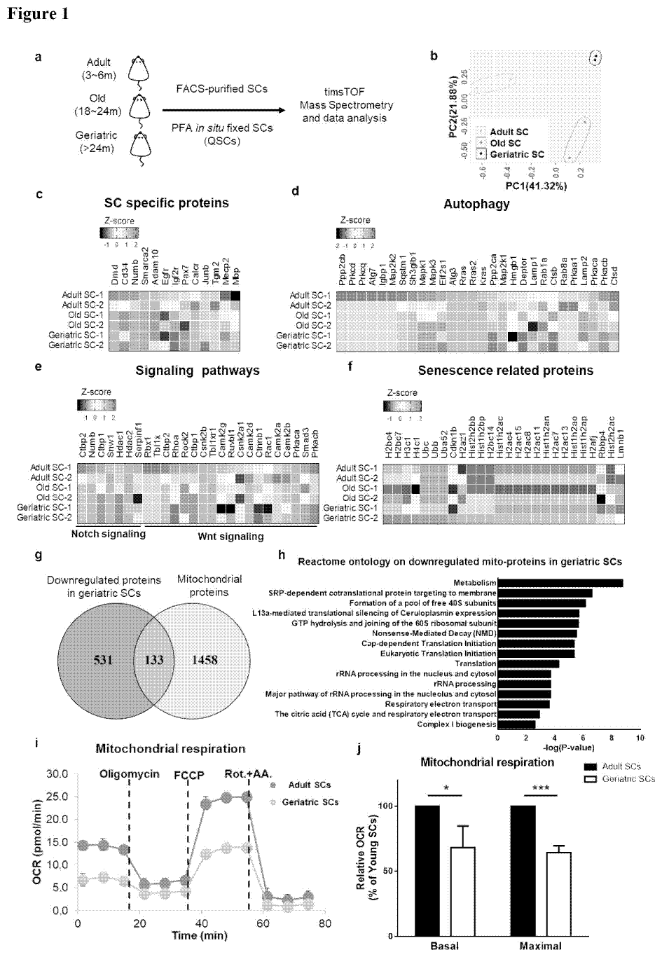

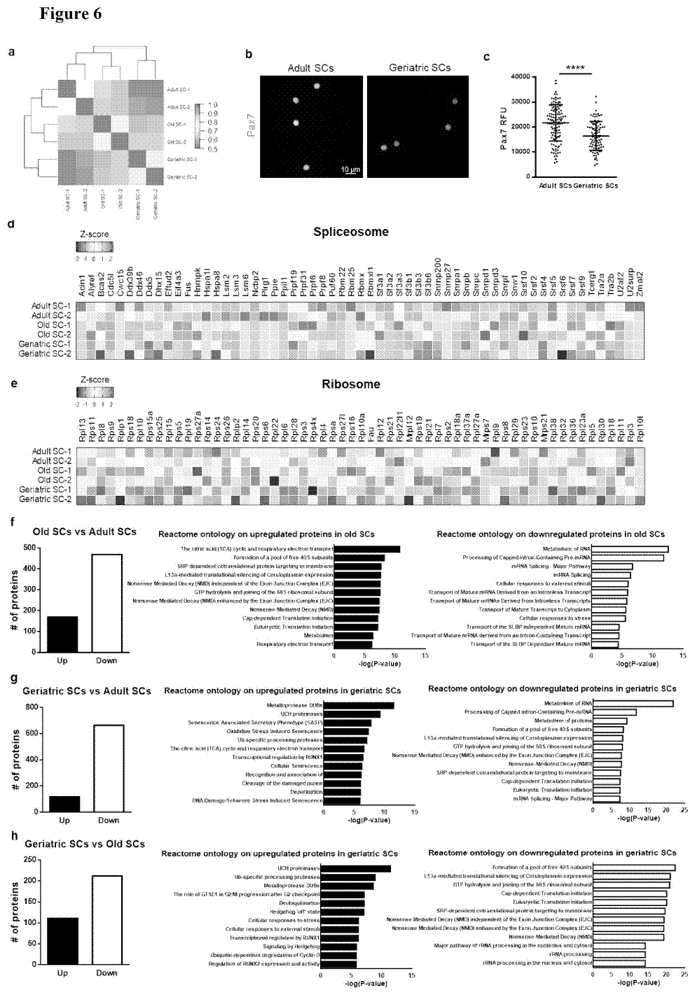

To uncover the mechanism of how SCs lose the capacity for activation and muscle repair during aging, we performed mass spectrometry and quantified the protein expression by label-free quantification methods (FIG. 1a). Principal component analysis (PCA) showed that adult, old and geriatric SCs are distinct from each other, but their corresponding biological replicates clustered closely, indicating the consistent reproducibility of the proteomic approach (FIG. 1b). From a whole proteome aspect, geriatric SCs are more distant from the adult SCs while old SCs lie between adult and geriatric SCs, indicating a gradual change of the proteomic landscape during aging (FIG. 6a). The SC-specific proteins such as Numb, Calcr and Pax7 were downregulated during aging (FIG. 1c, the expression level of Pax7 protein evaluated by immunostaining, as shown in FIGS. 6b, c). Autophagy was also impaired in geriatric SCs (Garcia-Prat et al., 2016) evidenced by a decline in the expression of autophagy-associated proteins (FIG. 1d). Past studies reported the SC niche is altered with reduced levels of Notch signaling related-proteins during aging (Liu et al., 2018). Indeed, we observed a reduced level of Notch and Wnt signaling-related components (FIG. 1e). Intriguingly, we also revealed that senescence-related proteins were only expressed in geriatric SCs (FIG. 1f). Moreover, we discovered that spliceosome-related proteins are already downregulated in old SCs whilst ribosome-related proteins were only significantly decreased in geriatric SCs (FIGS. 6d, e), indicating a gradual dysfunction in transcription and translation machinery during aging. Taken together, our proteomic landscape indicates that SC enters senescence in geriatric mice with a declined capacity for transcription and translation.

Through pair-wise comparisons of adult, old and geriatric SCs, we identified differentially expressed proteins of various pathways during aging. Translation-related pathways such as “Formation of a pool of free 40S subunits” were enriched in old SCs compared to adult SCs while transcription-related pathways such as “mRNA splicing” were enriched in adult SCs (FIG. 6f). Compared to adult SCs, the upregulated proteins in geriatric SCs were related to senescence pathways while the downregulated proteins were transcription and translation-related (FIG. 6g). We found that senescence pathways were enriched in geriatric SCs whilst translation-related pathways were enriched in old SCs (FIG. 6h). This indicates that old SCs may compensate for the reduced transcription capacity by boosting protein translation. Furthermore, we also observed that the Nonsense-Mediated Decay (NMD) pathway was enriched in old SCs when compared to adult SCs and also comparatively more enriched in adult SCs in comparison to geriatric SCs, indicating that NMD, whose loss results in dysfunctional protein aggregation, was boosted in old SCs but lost in geriatric SCs (FIGS. 6f-h). Collectively, our proteomic landscape indicates that SCs harbors the capacity to reduce aging defects before entering irreversible senescence.

### Example 2. Mitochondrial Defects in Geriatric SCs

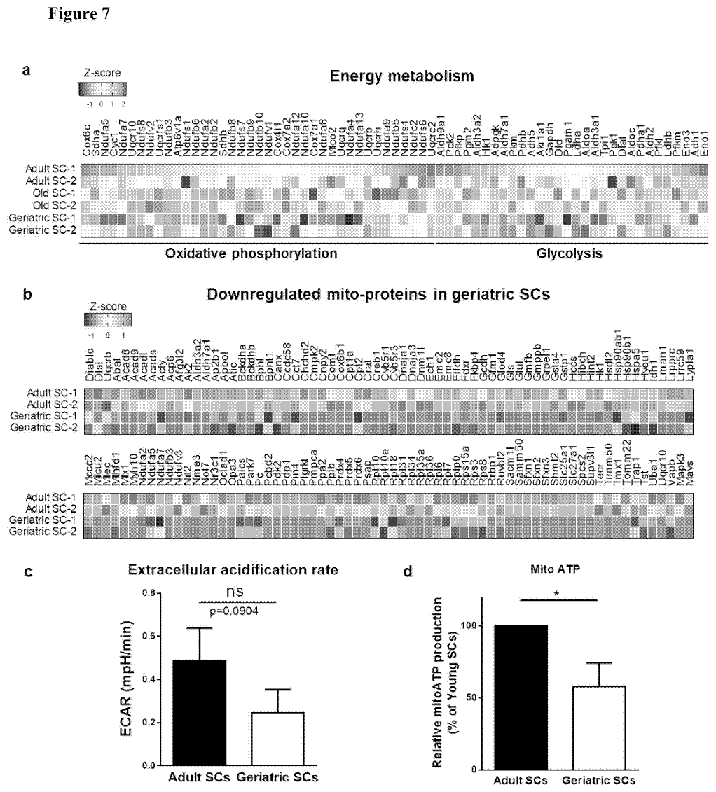

Intriguingly, we observed that energy metabolism pathways were dysregulated during aging. Oxidative phosphorylation and glycolysis-related proteins were decreased during aging (FIG. 7a). Mitochondria are the center for energy metabolism so to study mitochondrial proteins, we compared the differentially expressed proteins during aging between adult, old and geriatric SCs. 133 mitochondrial proteins were downregulated in geriatric SCs (FIG. 1g and FIG. 7b) and were enriched in pathways related to mitochondrial translation and energy metabolism (FIG. 1h). To examine whether mitochondrial activity was affected during aging, we performed live cell flux analysis. Consistent with the proteomic changes, we observed a dramatic decrease in the basal and maximal oxygen consumption rate (OCR) while the extracellular acidification rate (ECAR) remained unchanged (FIGS. 1i, j and FIG. 7c). We also found that mitochondrial ATP generation was decreased in geriatric SCs (FIG. 7d). Taken together, our data indicated that the mitochondrial proteome and activity are impaired in geriatric SCs.

### Example 3: CPEB4 is Lost During Aging and Regulates SC Function

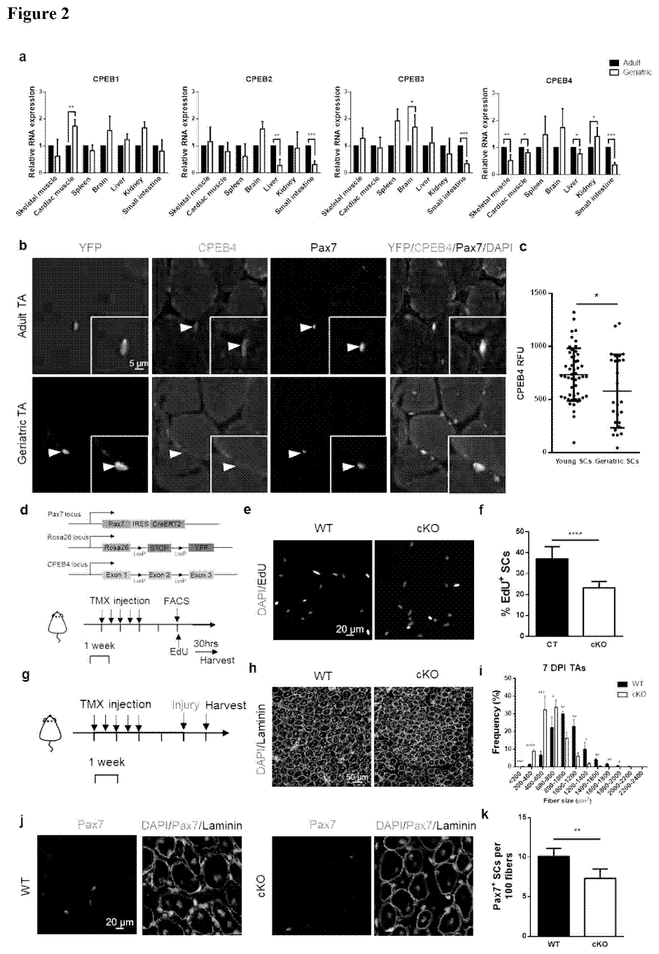

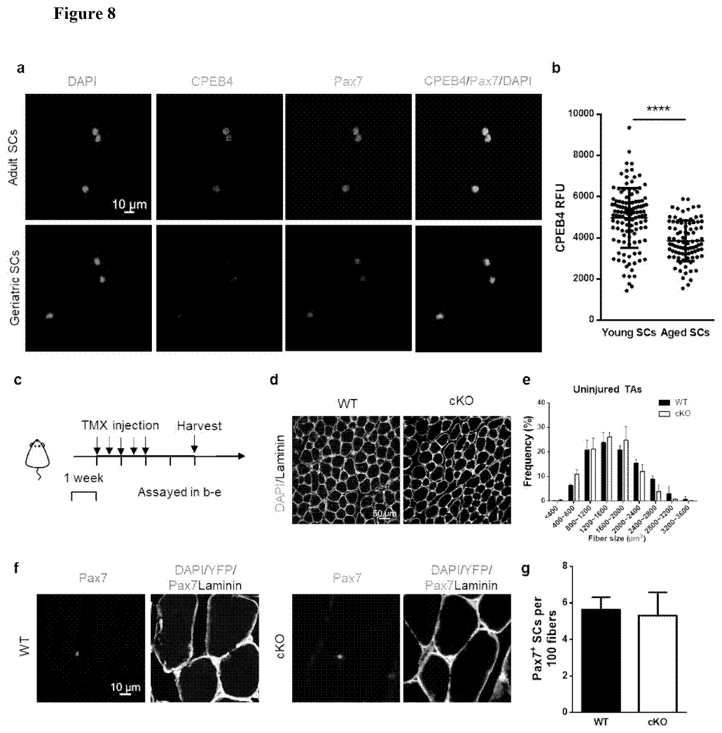

To investigate the mechanism behind the changes in the proteome during aging, we focused on the CPEB family of proteins, which regulate various cellular functions including cancer development, cell cycle regulation, stem cell differentiation, and tissue homeostasis by translational control. We observed that the expression levels of CPEB1-3 mRNA remain largely unchanged while CPEB4 mRNA levels are significantly decreased during aging in tissues such as skeletal muscle, cardiac muscle, liver, and small intestine (FIG. 2a). We next examined CPEB4 protein expression during SC aging, by immunostaining for CPEB4 in adult and geriatric SCs and found that CPEB4 protein is lost during aging (FIGS. 2b, c and FIGS. 8a, b).

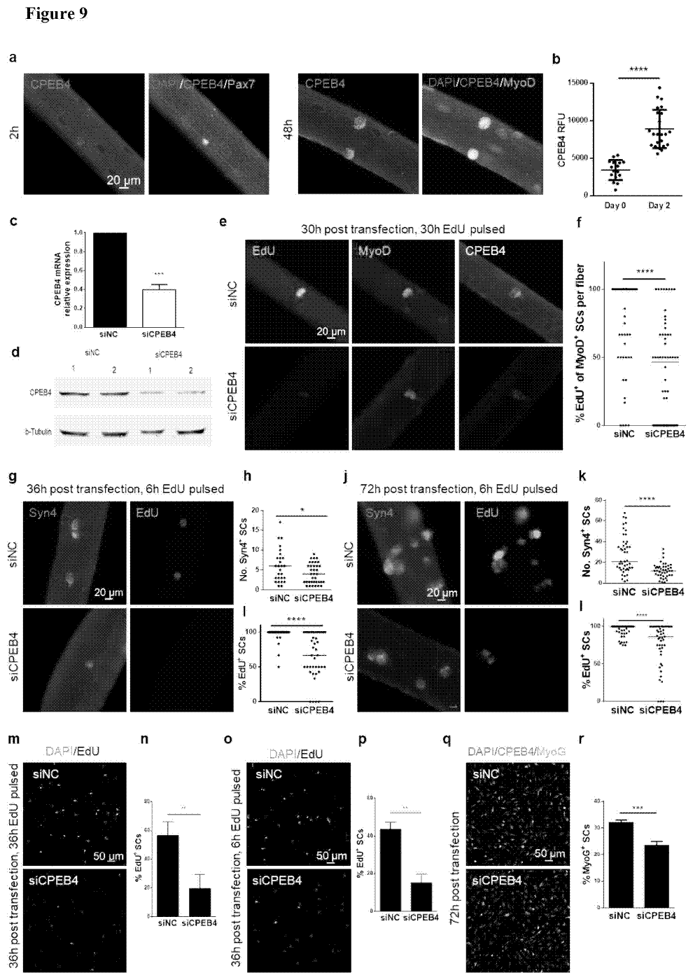

To investigate the roles of CPEB4 in SC function, we utilized a loss-of-function siRNA-mediated approach. Immunostaining results showed that CPEB4 protein is upregulated during SC activation (FIGS. 9a, b). Knocking down CPEB4 resulted in the delay of SC activation shown by the reduced percentage of EdU⁺ SCs (FIGS. 9e, f, m, n; EdU added to mark cycling SCs, the efficiency and specificity of siRNA for CPEB4 shown in FIGS. 9c, d), indicating that CPEB4 is essential for SC activation. Moreover, SC proliferation was also affected, as demonstrated by the decreased SC number in single myofibers (Syn4 marks both quiescent SCs and activated SCs) (FIGS. 9g-l) and reduced EdU incorporation (SCs were pulsed with EdU for 6 hours before harvest). Similar results were achieved when using FACS-purified SCs (FIGS. 9o, p). We also evaluated the expression of the SC differentiation marker MyoG and found that the percentage of MyoG⁺ SCs was significantly decreased after CPEB4 knockdown (FIGS. 9q, r). Together, these data indicate that CPEB4 is essential for myogenic lineage progression.

Apart from the siRNA approach, we also investigated CPEB4 function using CPEB4 conditional knockout (cKO) mice. Consistent with CPEB4 siRNA knockdown data, the CPEB4 cKO SCs also showed delayed activation (FIGS. 2d-f). Staining of uninjured TA muscle sections showed that the fiber size was comparable between CPEB4 WT and cKO mice (FIGS. 8c-e). The numbers of Pax7⁺ SCs were also unchanged (FIGS. 8f, g). Taken together, SCs and muscle homeostasis remain functional under uninjured conditions after CPEB4 deletion. However, the size of newly regenerated myofibers was significantly smaller 7 days post-injury (FIGS. 2g-i) and the numbers of Pax7⁺ SCs was decreased after CPEB4 deletion (FIGS. 2j, k). Collectively, CPEB4 is required for muscle regeneration and stem cell pool maintenance after muscle injury.

### Example 4: CPEB4 Maintains Mitochondrial Function

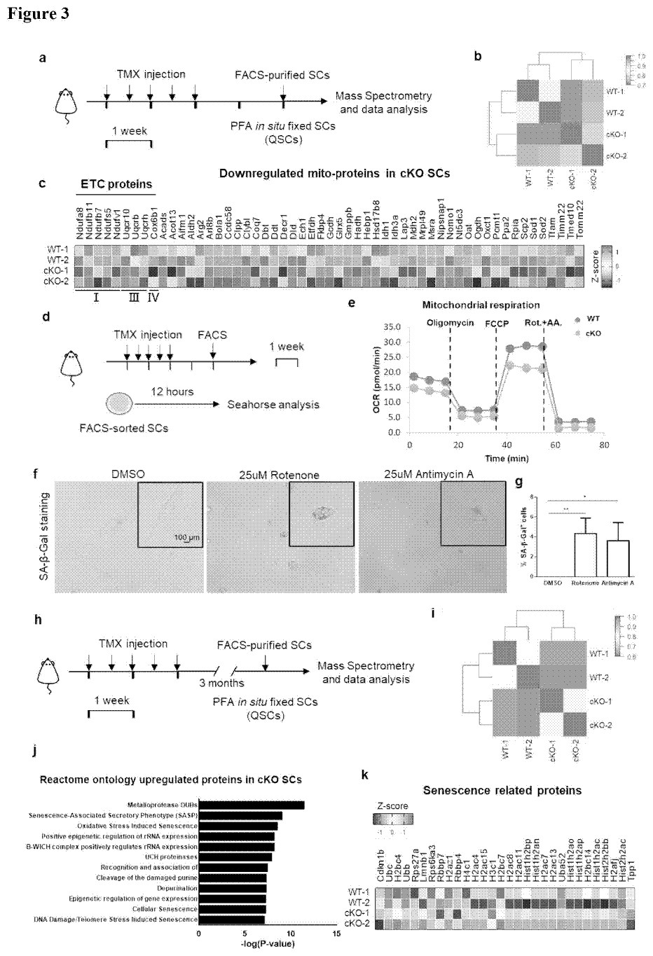

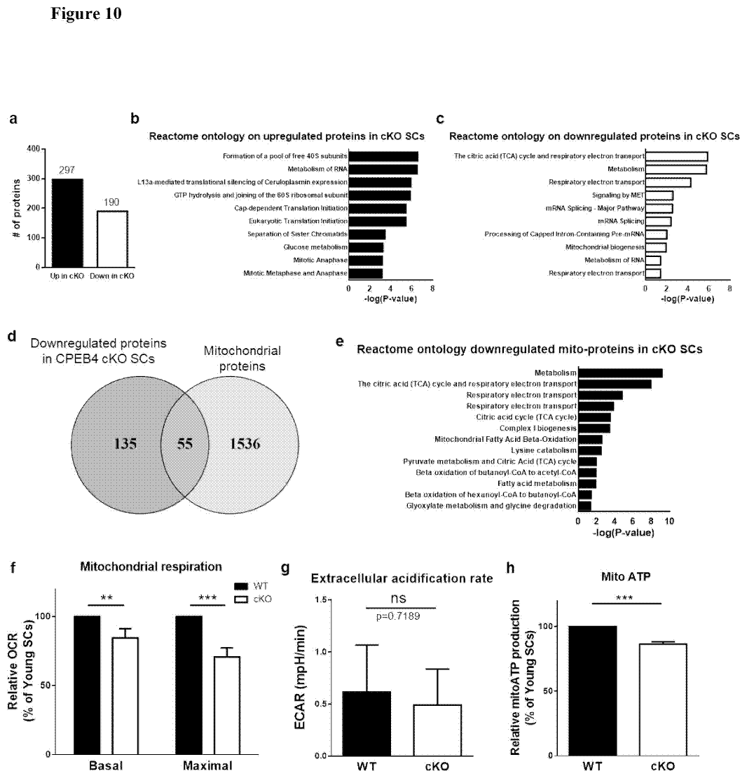

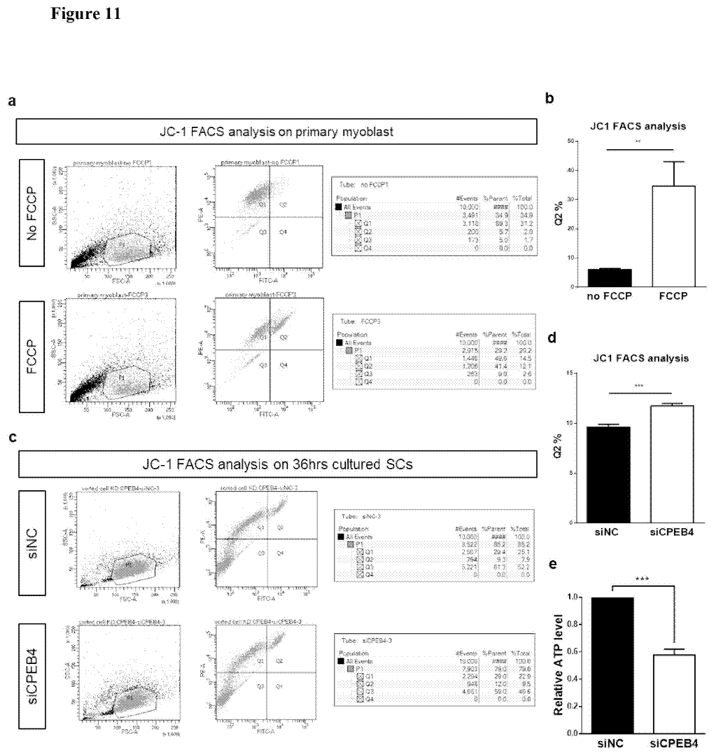

As to what role CPEB4 plays in SC function, we analyzed the proteomic landscape of CPEB4 cKO SCs (two weeks after Tamoxifen induction to knockout CPEB4) (FIG. 3a). From a whole proteome aspect, SCs were distant between CPEB4 WT controls and CPEB4 cKO SCs (FIG. 3b). We found that the upregulated proteins in CPEB4 cKO SCs were translation-related while downregulated proteins were related to transcription, TCA cycle, mitochondrial biogenesis and regulation of mitochondrial respiration (FIGS. 10a-c), indicating CPEB4 regulates mitochondrial energy metabolism. To determine whether CPEB4 deletion affects the mitochondrial proteomic landscape, we compared the differentially expressed proteins with the mitochondrial proteomic data. We identified 55 downregulated mitochondrial proteins in CPEB4 cKO SCs, including electron transport chain proteins (FIG. 3c and FIG. 10d). Functional enrichment analysis showed that these downregulated mitochondrial proteins were enriched in the TCA cycle and respiratory electron transport (FIG. 10e), which correlates with the observed mitochondrial activity defects (FIGS. 3d, e and FIGS. 10f-h). We also noted that the active mitochondria number decreased after CPEB4 knockdown using the JC-1 assay (mitochondrial depolarization is indicated by a decrease in the red/green fluorescence intensity ratio) (FIGS. 11a-e). Collectively, we show that CPEB4 is required for mitochondrial activity maintenance.

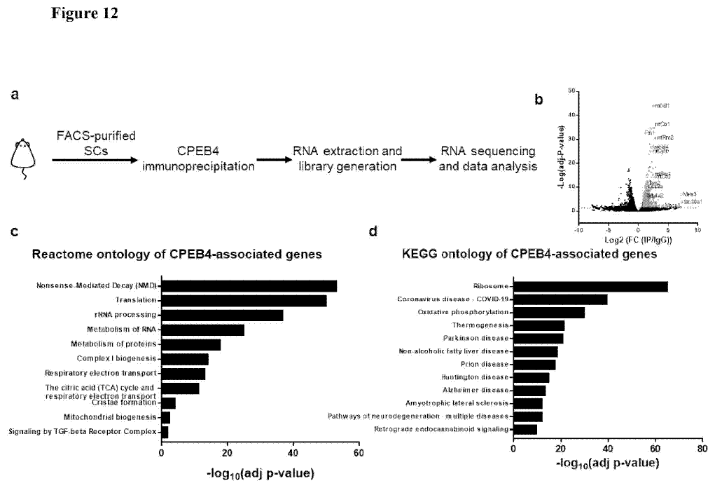

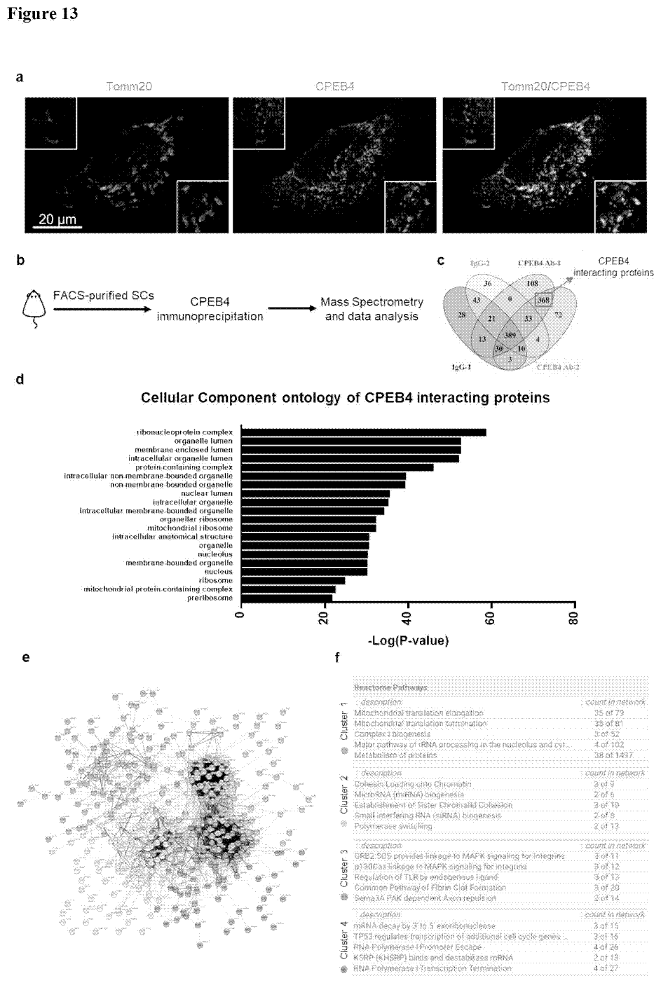

CPEB4 is an RNA binding protein that regulates translation through cytoplasmic polyadenylation of its bound transcripts. To identify the global CPEB4-targeted transcripts in SCs, we performed CPEB4 antibody immunoprecipitation followed by RNA isolation and sequencing (RIP-seq) (FIG. 12a). We found that CPEB4 binds to numerous transcripts encoding mitochondrial proteins such as Cox8a (FIG. 12b). Reactome ontology analysis revealed that CPEB4-associated genes enriched in “NMD”, “Translation”, “Complex I biogenesis” and “TCA cycle” in correlation with the proteomic landscape changes after CPEB4 deletion (FIG. 12c). Intriguingly, KEGG analysis showed that CPEB4-associated genes were enriched in “Coronavirus disease” and aged-related diseases such as Parkinson's and Alzheimer's disease (FIG. 12d). Moreover, super resolution structured illumination microscopy (SIM) on SCs showed that some of the CPEB4 protein is indeed in the mitochondria (FIG. 13a). To examine whether CPEB4 associates with mitochondrial translational machinery, we performed CPEB4 antibody immunoprecipitation followed by mass spectrometry (FIG. 13b). We identified 368 CPEB4 binding proteins (FIG. 13c) which are components of the mitochondrial ribosome or mitochondrial protein-containing complexes (FIG. 13d). Functional protein network analysis revealed that CPEB4-interacting proteins are clustered into 4 categories while the proteins in cluster 1 are mitochondrial translation elongation or termination-related proteins (FIGS. 13e, f). Taken together, CPEB4 is a key player in mitochondrial translation, targeting genes encoding mitochondrial proteins and interacting with the mitochondrial translation machinery.

### Example 5: Long-Term Loss of CPEB4 Induces Senescence

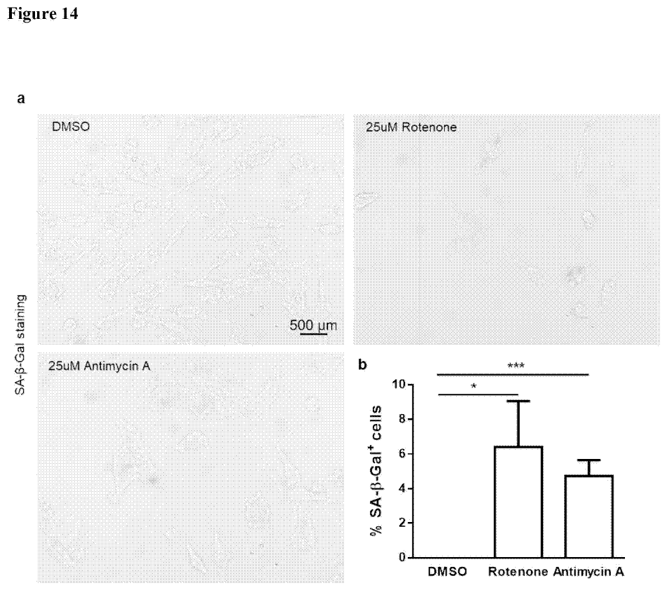

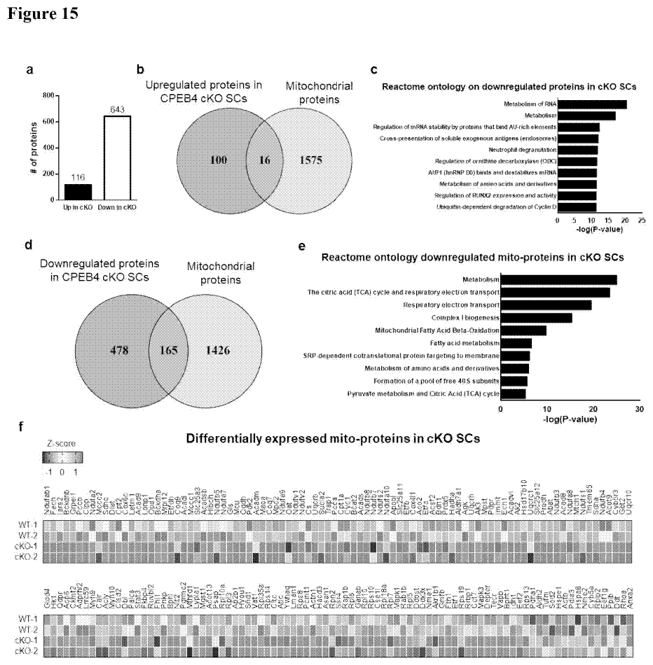

We observed that short-term CPEB4 deletion in SCs (two weeks after CPEB4 knockout induction) resulted in mitochondrial defects, also reflected in geriatric SCs. Moreover, CPEB4 was lost during aging, and geriatric SCs display a senescence-associated proteomics signature. Most importantly, other studies and our data showed that inhibiting mitochondrial metabolism results in cellular senescence (FIGS. 3f, g and FIG. 14). Thus, we hypothesize that long-term loss of CPEB4 induces senescence. To test this hypothesis, we performed mass spectrometry on CPEB4 long-term loss SCs (FIG. 3h, 3 months after induction of CPEB4 deletion, age of mice was 6 months). From a whole proteome aspect, SCs were distant between CPEB4 WT and cKO SCs (FIG. 3i). Expectedly, we found that the upregulated proteins in CPEB4 cKO SCs were enriched in senescence-related pathways such as “Oxidative Stress Induced Senescence” and “DNA Damage/Telomere Stress Induced Senescence” (FIGS. 3j, k). We also observed that the downregulated proteins after CPEB4 deletion are RNA metabolism or stability regulation and amino acid metabolism or translation-related (FIGS. 15a, c). Moreover, we found that 165 mitochondrial proteins were downregulated, enriched in the TCA cycle and mitochondrial complex I biogenesis, whilst only 16 mitochondrial proteins were upregulated following CPEB4 deletion (FIGS. 15b, d-f). Collectively, we demonstrated that long-term loss of CPEB4 results in a severely impaired mitochondrial proteomics landscape and induces a senescence-associated proteomics signature.

### Example 6: Reversal of Senescence by CPEB4

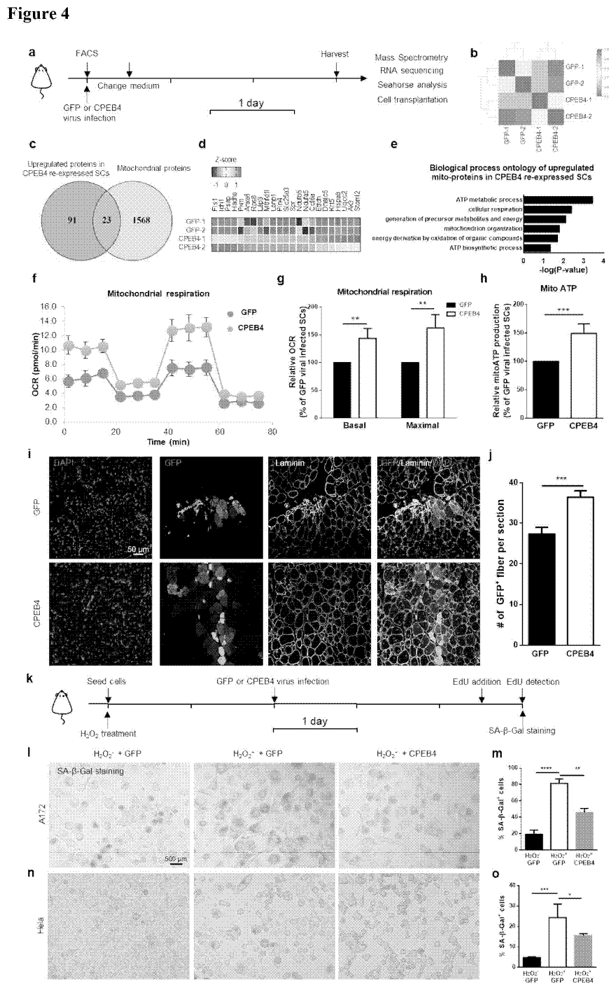

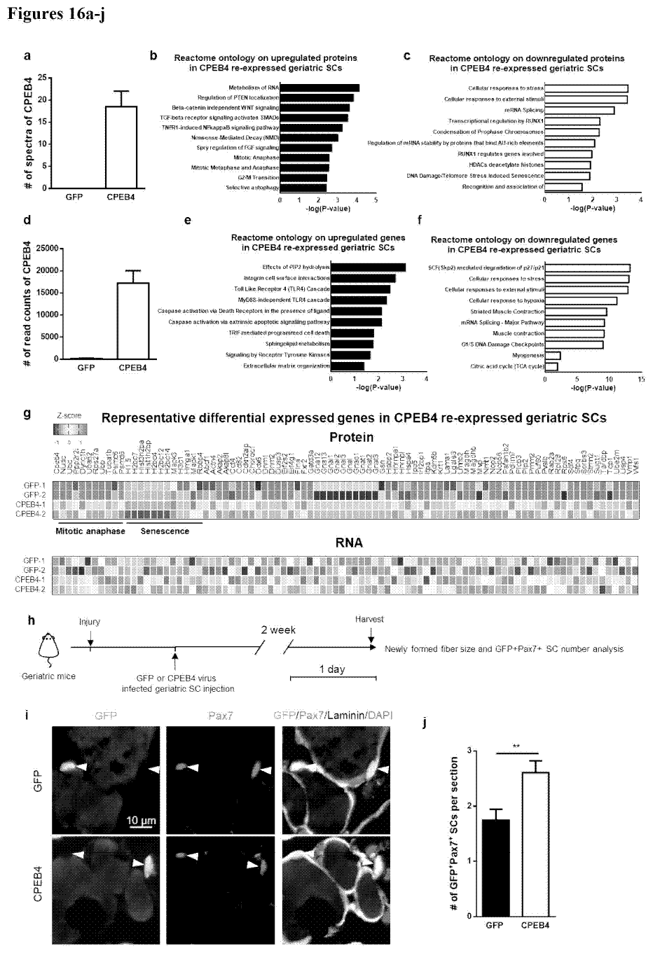

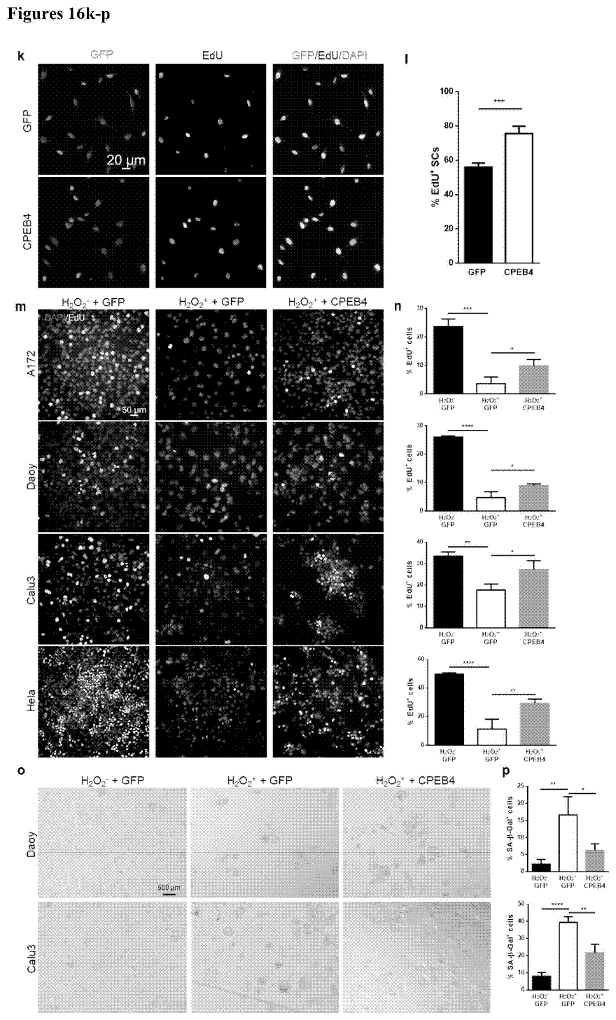

To examine whether restoring CPEB4 expression rejuvenates the geriatric SCs, we infected geriatric SCs with adenovirus containing the CPEB4 encoding sequence (FIG. 4a). The mass spectrometry data revealed that the proteomics landscape is distinct between control and CPEB4 overexpressing SCs (FIG. 4b). Reactome ontology analysis of differentially expressed proteins showed that upregulated proteins were enriched in cell cycle-related pathways such as mitotic anaphase while downregulated proteins were enriched in DNA damage/telomere stress-induced senescence pathways (FIGS. 16a-c, g). This observation is consistent with our data showing that loss of CPEB4 affects proliferation and induces senescence. Surprisingly, the enriched pathways of differentially expressed mRNAs identified by RNA-sequencing after CPEB4 overexpression differed highly from the pathways identified by mass spectrometry (FIGS. 16d-f), which confirms the disconcordance between RNA and protein expression (FIG. 16g). Moreover, this also reveals the importance of CPEB4 as a translational regulator. By comparing upregulated proteins after CPEB4 overexpression and the mitochondrial proteome, we identified 23 upregulated mitochondrial proteins after CPEB4 virus infection (FIGS. 4c, d). Such proteins were related to ATP biosynthesis (FIG. 4e). Furthermore, analysis of mitochondrial respiration showed that the basal and maximal OCR was significantly increased and that ATP production increased after CPEB4 viral infection of geriatric SCs (FIGS. 4f-h).

To investigate whether restoring CPEB4 expression could rescue the regenerative block and cell intrinsic irreversible cell cycle of geriatric SCs, we engrafted the CPEB4-infected geriatric YFP⁺ SCs (isolated from Pax7-CreER; Rosa26-YFP mice) into pre-injured muscles of geriatric recipient mice (FIG. 16h). The muscle graft experiments demonstrated that CPEB4 induction alone in geriatric SCs was sufficient to rescue their intrinsic regenerative capacity, allowing the formation of new muscle fibers (FIGS. 4i, j). The increase in Pax7⁺ GFP⁺ SC number indicated that CPEB4 restoration rescued SC self-renewal or niche homing capacity in geriatric SCs (FIGS. 16i, j). Furthermore, the cell expansion capacity was rescued by CPEB4 restoration, indicated by the increased EdU incorporation rate (FIGS. 16k, l). We also investigated whether CPEB4 overexpression alone could rescue cellular senescence in human cell lines. EdU incorporation showed that CPEB4 overexpression rescued hydrogen peroxide (H₂O₂)-induced cell cycle defects in several human cell lines (FIGS. 16m, n). Moreover, SA-β-Gal staining demonstrated that CPEB4 overexpression prevented H₂O₂-induced cellular senescence in human cell lines (FIGS. 4l-o and FIGS. 16o, p).

### Methods

### Animals and Treatments

Adult (3 to 6 months), old (18 to 24 months), and geriatric mice (>24 months) used in this study were housed and maintained in the Laboratory Animal Facility (LAF) at HKUST. The definition of mouse age followed the Jackson Laboratory criteria. All animal experiments were approved by the HKUST Animal Ethics Committee, and all studies used male mice for each experiment, with the control and experimental mice being age-matched. C57BL/6 mice were obtained from the LAF. Pax7CreERT2/+; CPEB41oxp/loxp; ROSA26YFP/+ were generated by crossing Pax7CreERT2/+ mice (Murphy et al., 2011) with CPEB41oxp/loxp or ROSA26YFP/+ mice. Genotyping primers sequences are listed in the Supplementary information. Tamoxifen (TAM) injections for Cre recombinase activation was performed via intraperitoneal injection into the mouse in a dosage of 0.2 mg of TAM per gram of mouse, with a frequency of one injection every three days, for a total of 5 injections (Nishijo et al., 2009). Pax7CreERT2/+; CPEB4+/+; ROSA26YFP/+ mice were used as controls in this study unless noted otherwise. For whole mouse perfusion, please refer to previous studies performed in our laboratory for detailed methods (Yue and Cheung, 2020; Yue et al., 2020). For anesthesia, the mice were anesthetized by intraperitoneal (IP) injection of tribromoethanol (Avertin) at a dosage of 500 mg/kg. For euthanasia, mice were sacrificed by carbon dioxide (CO₂) inhalation followed by cervical dislocation.

### Muscle Injury

Before injury, the mice were anesthetized by IP injection of tribromoethanol. TA muscles were injured by injecting 30 μL of 1.2% Barium Chloride (BaCl2, Sigma-Aldrich). Shank muscles were injured for activated SC sorting by evenly injecting 50 μL of 1.2% BaCl₂, followed by even stabbing using a 31G insulin syringe (BD Biosciences) approximately 50 times.

Cell Culture and siRNA Transfection

Freshly isolated SCs were cultured in Ham's F10 (F10, Sigma-Aldrich), 10% horse serum (HS, Invitrogen) and penicillin-streptomycin (1% P/S, Invitrogen). Medium was replenished every 24 hours. For siRNA transfection (RiboBio), Lipofectamine 3000 reagent (Invitrogen) was used according to the manufacturer's instructions. The siRNA sequences are listed in the oligo sequence table. The effect of siRNA-mediated knockdown was performed on cultured SCs.

### Satellite Cell Isolation

Briefly, hindlimb muscles were dissected and digested in wash medium (F10, 10% HS, P/S) containing collagenase II (1000 U/mL) for 90 minutes. The tissue slurry was further digested with collagenase II (100 U/mL) and dispase (1 U/mL) for 30 minutes to obtain a single cell suspension for cell sorting. The detailed protocol was as previously described (Liu et al., 2015). For SC isolation from the whole PFA-perfused mouse, collagenase II (2,000 U/mL) was used in the first digestion. The cells were sorted using the BD Influx cell sorter equipped with 405-nm, 488-nm, 561-nm and 633-nm lasers. The machine was carefully optimized for purity and viability. To assess the purity, a small fraction of sorted cells were subjected to FACS analysis and immunofluorescence staining for the myogenic markers Pax7 and Myod1.

### Single Fiber Explants Isolation

Mouse Extensor digitorum longus (EDL) muscles were excised and digested with collagenase II (800 U/mL) in wash medium (F10, 10% HS, P/S) for 80 minutes to obtain single fibers. Single fibers were then carefully washed and placed in wash medium (Cheung et al., 2012). For transfection of siRNA (RiboBio), Lipofectamine 3000 (Invitrogen) was used according to the manufacturer's instructions.

### Cloning

The CPEB4 coding sequence (CDS) was cloned into the W35-pENTR entry plasmid by Gibson Assembly (NEB) for adenovirus packaging. The entry vector was recombined into the pAd/BLOCKitDEST vector using Clonase II (Invitrogen) according to the manufacturer's instructions. The correct clone was confirmed by sequencing.

### EdU Incorporation Analysis

For the EdU incorporation assay, freshly isolated SCs were constantly supplied with M of EdU (Invitrogen) during culture until harvest or 6 hours before harvest. Cells were then fixed and stained using the Click-iT EdU Imaging Kit (Invitrogen) according to the manufacturer's instructions.

### Histology and Immunohistochemistry

Tibialis anterior (TA) muscles were first fixed in 0.5% PFA for 6 hours and then incubated in 20% sucrose (Sigma-Aldrich) overnight. Muscles were then frozen in optimal cutting temperature compound (OCT, Tissue-Tek), cryo-sectioned at a thickness of 6 m using the Cryostat NX 70 (Thermo), and stained using Fab antibodies (Jackson ImmunoResearch) or an APEX Antibody Labelling Kit (Invitrogen) according to the manufacturer's instructions.

### Immunofluorescence

Images were viewed using the Zeiss Axio Observer Z1 fluorescent microscope (Zeiss) equipped with a Hamamatsu ORCA-ER camera. Super-resolution images were taken on the Elyra 7 system equipped with Structured Illumination Microscopy (SIM). Data acquisition was performed using the ZEN software (blue edition, Zeiss).

### CPEB4 RNA Immunoprecipitation Sequencing and Data Analysis

1×10⁶ freshly isolated SCs were harvested in polysome lysis buffer (PLB) with protease inhibitor (Sigma) and RNase inhibitor (Invitrogen). The lysate was precleared using Protein A Dynabeads (ThermoFisher Scientific). CPEB4 antibody (10 μg, Proteintech) was added to the precleared lysate and incubated with overnight rotation at 4° C. Protein A Dynabeads were resuspended in PLB and added to the SC lysate and rotated at 4° C. for 4 hours. The lysate was put on a magnetic stand and the supernatant discarded. Beads were washed four times in PLB and rotated at 4° C. for 5 minutes per wash. RNA was isolated using the Nucleospin RNA XS kit (Machery Nagel), and the cDNA library was generated using the SMART-Seq2 method followed by MGI 2000 sequencing machine using the 2×100 kit. Raw reads were mapped to the mm10 genome using STAR (Dobin et al., 2013). Reads were assembled and quantified by Featurecounts (Liao et al., 2014). The raw read counts were used to perform DEseq2 (Love et al., 2014) analysis to determine the CPEB4-enriched genes. The volcano plot of CPEB4 binding genes was generated using GraphPad 7. Gene ontology (GO) analysis was performed using g:Profiler (Raudvere et al., 2019).

Real-Time qPCR

Total RNA was isolated using the NucleoSpin RNA XS kit (Macherey-Nagel). Isolated RNA was quantified using the Qubit fluorometer (ThermoFisher Scientific). cDNA was generated using the High-Capacity cDNA Reverse Transcription Kit (Invitrogen). Real-time PCR reactions were set up in volumes of 10 μL containing 5 μL 2×SYBR Master Mix (Roche), 4 μL cDNA template, and 1 μL 375 nM primer. Real-time PCR was performed using the Light-Cycler 480 (Roche).

### Western Blot

Cells were lysed in RIPA buffer with complete EDTA-free Protease Inhibitor Cocktail (Roche) and boiled for 5 minutes at 98° C. 30 μg of protein extracts were electrophoresed on 10-15% polyacrylamide gradient gels and then transferred onto nitrocellulose membranes (Bio-Rad). Membranes were incubated in blocking buffer (5% milk in 0.05% TBST) for 1 hour before overnight incubation with primary antibodies. The antibodies used for Western blotting were anti-CPEB4 (ProteinTech Group, 1: 2000) and anti-Glyceraldehyde-3-phosphate dehydrogenase (GAPDH) (UBC, 1:5,000, loading control). After incubation with the corresponding fluorescent secondary antibodies (Invitrogen, 1:10,000), protein bands were analyzed using the Odyssey Imaging System (LI-COR Biosciences).

### Adenovirus Packaging and Infection

To generate adenovirus, the Block-iT Adenoviral RNAi Expression System (Invitrogen) was used. Briefly, 293A packaging cells cultured to 90% confluency in 6-well plates were transfected with 1 μg pAd/BLOCKiT-DEST vectors carrying GFP or CPEB4-GFP using Lipofectamine 2000. After 12 days in culture, viral supernatant was harvested for subsequent amplification and concentration. The titer of adenovirus was approximately 1×10⁷ pfu/ml. SCs were infected with adenovirus-containing medium for 12 hours. The experimental design for adenovirus infection of cells for mass spectrometry, RNA-sequencing, seahorse analysis and SC transplantation are described in the corresponding figure legends.

### Mass Spectrometry and Label-Free Quantification of Mass Spectrometry Data

SC-derived proteins were extracted in RIPA buffer. For prefixed QSCs, the protein lysate was heated at 70° C. for 2 hours to de-crosslink the fixed proteins, and then the protein precipitated by adding 4× volume of pre-cooled acetone (Honeywell). The protein pellet was sequentially washed with pre-cooled acetone, pre-cooled ethanol, and then pre-cooled acetone. The pellet was resuspended with UA buffer (8 M urea in 0.1 M Tris-HCl), and then dithioreitol (DTT, 2 mM) was added before incubation at 30° C. for 1.5 hours. Iodoacetamide (IAA) (Sigma-Aldrich, 10 mM) was added to the sample, and the sample was incubated protected from light for 40 minutes. Afterward, trypsin was added (final concentration 0.25 μg/μl) to the suspension for overnight digestion. The digestion was stopped by adding trifluoroacetic (TFA) (Sigma-Aldrich, final concentration 0.4%). After salt depletion using C18 spin tips (Thermo Scientific), the material was loaded onto the Bruker timsTOF Pro mass spectrometer following the manufacturer's instructions. For Liquid chromatography (LC), the mobile phase A was 98% MilliQ Water, 2% Acetonitrile with 0.1% Formic Acid, and the mobile phase B was 100% Acetonitrile with 0.1% Formic acid. We used the ionoptiks 25 cm Aurora Series emitter column with CSI (25 cm×75 μm ID, 1.6 μm C18).

The raw data was processed by PEAKS software (Version: X+). The database for searching the proteomic data was Uniprot, and the taxonomy was Mus musculus. The parent ion was 15 ppm, and the fragment ion was 0.05 Da. The protein FDR was 1%. We applied spectral counting for label-free quantification, followed by the normalized spectral abundance factor (NSAF) method (Zhu et al., 2010). We first obtained the spectral abundance factor by normalizing the spectral number of proteins with the length of the protein and then normalized the spectral abundance factor between samples by dividing by the sum of all the spectral abundance factors. As the NSAF was very small, we multiplied the NSAF by 10⁶. The student's t-test was performed to determine the differentially expressed proteins between samples. We used one-pair distribution and a homoscedastic Student's t-test. The level of statistical significance was set at p<0.05.

### Measurement of OCR, ECAR and ATP Production

The oxygen consumption rate (OCR) and extracellular acidification rate (ECAR) were determined using the Seahorse XFp analyzer (Agilent) using the “Cell Mito Stress” test kit. Briefly, 2×10⁴ of freshly isolated SCs were seeded into each well of a miniplate and cultured in growth medium (F10 with 10% horse serum). One hour prior to assaying, cells were first incubated with XF DMEM (pH 7.4) medium (supplemented with 1 mM of pyruvate (Gibco) 1 mM of glutamine (Gibco), and 1 g/L of glucose (Sigma-Aldrich)) in a CO₂-free incubator at 37° C. The “Cell Mito Stress” assay was performed following the manufacturer's instructions. ATP production rates were calculated using the methodology outlined in “Quantifying Cellular ATP Production Rate Using Agilent Seahorse XF Technology.”

### CPEB4 Immunoprecipitation for Mass Spectrometry

1×10⁶ freshly isolated SCs were harvested in PLB for CPEB4 immunoprecipitation following the steps described for CPEB4 RIP-sequencing. Protein was eluted by adding 100 μl RIPA buffer to the beads for 10 minutes at 95° C. The protein was precipitated with acetone for mass spectrometry analysis. To reduce the number of false positive results, we only considered the proteins that were detected in the CPEB4 antibody IP group but not in the IgG control group.

### SC Transplantation

1×10⁵ freshly isolated SCs from geriatric Pax7-YFP mice (aged 26 months) were seeded into a 6-well culture plate followed by CPEB4-containing adenovirus infection for 12 hours. Three days after SC culturing, infected SCs were trypsinized into a single cell suspension followed by centrifugation to form a cell pellet. The cell pellet was resuspended in phosphate-buffered saline (PBS, Gibco). 1×10⁴ SCs were transplanted into one day pre-injured TA muscles of geriatric mice (aged 30 months). Two weeks following transplantation, the TA muscles were harvested to determine the fiber size and GFP⁺ Pax7⁺ SC number.

### Reversal of Cellular Senescence by CPEB4 Viral Infection

3×10³ cells of different cell lines were seeded each well of a 96-well plate and treated with 50 μM hydrogen peroxide (H₂O₂, Sigma-Aldrich). Two days following treatment, the cells were infected with adenovirus containing the CPEB4 coding sequence at an MOI of 10 for 12 hours. Three days post-infection, the cells were harvested for senescence-associated beta-galactosidase (SA-β-Gal) staining. For measuring EdU incorporation, the cells were pulsed with 10 μM EdU for 12 hours, followed by EdU detection.

### Quantification and Statistical Analysis

All statistical analyses were performed using GraphPad Prism 7 (GraphPad Software) or R (version 3.6.1). All replicates in this manuscript are biological replicates, indicated by n. Unless otherwise noted, all error bars indicate the standard deviation (SD). Immunofluorescent images were quantified and analyzed using ZEN Lite 2.5, and the relative fluorescence units were quantified by determining the mean of the fluorescence signal. No statistical methods were used to predetermine the sample size. Experiments were not randomized, and the investigators were not blinded to allocation during experiments and outcome assessment.

All patents, patent applications, and other publications, including GenBank Accession Numbers, cited in this application are incorporated by reference in the entirety for all purposes.

### REFERENCES

Ahlqvist, K. J., Suomalainen, A., and Hämäläinen, R. H. (2015). Stem cells, mitochondria and aging. Biochim. Biophys. Acta BBA—Bioenerg. 1847, 1380-1386.
Alt, E. U., Senst, C., Murthy, S. N., Slakey, D. P., Dupin, C. L., Chaffin, A. E., Kadowitz, P. J., and Izadpanah, R. (2012). Aging alters tissue resident mesenchymal stem cell properties. Stem Cell Res. 8, 215-225.
Ansó, E., Weinberg, S. E., Diebold, L. P., Thompson, B. J., Malinge, S., Schumacker, P. T., Liu, X., Zhang, Y., Shao, Z., Steadman, M., et al. (2017). The mitochondrial respiratory chain is essential for haematopoietic stem cell function. Nat. Cell Biol. 19, 614-625.
Baek, D., Villén, J., Shin, C., Camargo, F. D., Gygi, S. P., and Bartel, D. P. (2008). The impact of microRNAs on protein output. Nature 455, 64-71.
Bava, F.-A., Eliscovich, C., Ferreira, P. G., Miñana, B., Ben-Dov, C., Guigó, R., Valcárcel, J., and Méndez, R. (2013). CPEB1 coordinates alternative 3′-UTR formation with translational regulation. Nature 495, 121-125.
Behrens, A., van Deursen, J. M., Rudolph, K. L., and Schumacher, B. (2014). Impact of genomic damage and ageing on stem cell function. Nat. Cell Biol. 16, 201-207.
Bhattacharya, D., and Scimè, A. (2020). Mitochondrial Function in Muscle Stem Cell Fates. Front. Cell Dev. Biol. 8.
Brack, A. S., and Muñoz-Cánoves, P. (2015). The ins and outs of muscle stem cell aging. Skelet. Muscle 6, 1.
Brett, J. O., Arjona, M., Ikeda, M., Quarta, M., de Morrée, A., Egner, I. M., Perandini, L. A., Ishak, H. D., Goshayeshi, A., Benjamin, D. I., et al. (2020). Exercise rejuvenates quiescent skeletal muscle stem cells in old mice through restoration of Cyclin D1. Nat. Metab. 2, 307-317.
Butow, R. A., and Avadhani, N. G. (2004). Mitochondrial Signaling: The Retrograde Response. Mol. Cell 14, 1-15.
Calderone, V., Gallego, J., Fernandez-Miranda, G., Garcia-Pras, E., Maillo, C., Berzigotti, A., Mejias, M., Bava, F.-A., Angulo-Urarte, A., Graupera, M., et al. (2016). Sequential Functions of CPEB1 and CPEB4 Regulate Pathologic Expression of Vascular Endothelial Growth Factor and Angiogenesis in Chronic Liver Disease. Gastroenterology 150, 982-997.e30.
Cao, G., Chen, D., Liu, G., Pan, Y., and Liu, Q. (2018). CPEB4 promotes growth and metastasis of gastric cancer cells via ZEB1-mediated epithelial&ndash; mesenchymal transition.
Chandel, N. S. (2014). Mitochondria as signaling organelles. BMC Biol. 12, 34.
Chandel, N. S., Jasper, H., Ho, T. T., and Passegue, E. (2016). Metabolic regulation of stem cell function in tissue homeostasis and organismal ageing. Nat. Cell Biol. 18, 823-832.
Chen, P.-J., and Huang, Y.-S. (2012). CPEB2-eEF2 interaction impedes HIF-1α RNA translation. EMBO J. 31, 959-971.
Cheung, T. H., and Rando, T. A. (2013). Molecular regulation of stem cell quiescence. Nat. Rev. Mol. Cell Biol. 14.
Cheung, T. H., Quach, N. L., Charville, G. W., Liu, L., Park, L., Edalati, A., Yoo, B., Hoang, P., and Rando, T. A. (2012). Maintenance of muscle stem-cell quiescence by microRNA-489. Nature 482, 524-528.
Chistiakov, D. A., Sobenin, I. A., Revin, V. V., Orekhov, A. N., and Bobryshev, Y. V. (2014). Mitochondrial Aging and Age-Related Dysfunction of Mitochondria. BioMed Res. Int. 2014, e238463.
Cho, J., Hur, J. H., and Walker, D. W. (2011). The Role of Mitochondria in Drosophila Aging. Exp. Gerontol. 46, 331-334.
Cogliati, S., Enriquez, J. A., and Scorrano, L. (2016). Mitochondrial Cristae: Where Beauty Meets Functionality. Trends Biochem. Sci. 41, 261-273.
Conboy, I. M., and Rando, T. A. (2005). Aging, stem cells and tissue regeneration: lessons from muscle. Cell Cycle Georget. Tex 4, 407-410.
Dobin, A., Davis, C. A., Schlesinger, F., Drenkow, J., Zaleski, C., Jha, S., Batut, P., Chaisson, M., and Gingeras, T. R. (2013). STAR: ultrafast universal RNA-seq aligner. Bioinforma. Oxf. Engl. 29, 15-21.
D'Souza, A. R., and Minczuk, M. (2018). Mitochondrial transcription and translation: overview. Essays Biochem. 62, 309-320.
Eliyahu, E., Pnueli, L., Melamed, D., Scherrer, T., Gerber, A. P., Pines, O., Rapaport, D., and Arava, Y. (2010). Tom20 Mediates Localization of mRNAs to Mitochondria in a Translation-Dependent Manner. Mol. Cell. Biol. 30, 284-294.
Fernández-Miranda, G., and Méndez, R. (2012). The CPEB-family of proteins, translational control in senescence and cancer. Ageing Res. Rev. 11, 460-472.
Ford, L., Ling, E., Kandel, E. R., and Fioriti, L. (2019). CPEB3 inhibits translation of mRNA targets by localizing them to P bodies. Proc. Natl. Acad. Sci. 116, 18078-18087.
Friedman, J. R., and Nunnari, J. (2014). Mitochondrial form and function. Nature 505, 335-343.
García-Prat, L., Martinez-Vicente, M., Perdiguero, E., Ortet, L., Rodríguez-Ubreva, J., Rebollo, E., Ruiz-Bonilla, V., Gutarra, S., Ballestar, E., Serrano, A. L., et al. (2016). Autophagy maintains stemness by preventing senescence. Nature 529, 37-42.
Ge, Y., Miao, Y., Gur-Cohen, S., Gomez, N., Yang, H., Nikolova, M., Polak, L., Hu, Y., Verma, A., Elemento, O., et al. (2020). The aging skin microenvironment dictates stem cell behavior. Proc. Natl. Acad. Sci. 117, 5339-5350.
Guha, M., Srinivasan, S., Ruthel, G., Kashina, A. K., Carstens, R. P., Mendoza, A., Khanna, C., Van Winkle, T., and Avadhani, N. G. (2014). Mitochondrial retrograde signaling induces epithelial-mesenchymal transition and generates breast cancer stem cells. Oncogene 33, 5238-5250.
Haas, R. H. (2019). Mitochondrial Dysfunction in Aging and Diseases of Aging. Biology 8.
Hu, J., Zhang, L., Chen, Q., Lin, J., Wang, S., Liu, R., Zhang, W., Miao, K., and Shou, T. (2018). Knockdown of CPEB4 expression suppresses cell migration and invasion via Akt pathway in non-small cell lung cancer. Cell Biol. Int. 42, 1484-1491.
Huang, Y.-S., Kan, M.-C., Lin, C.-L., and Richter, J. D. (2006). CPEB3 and CPEB4 in neurons: analysis of RNA-binding specificity and translational control of AMPA receptor GluR2 mRNA. EMBO J. 25, 4865-4876.
Khacho, M., Clark, A., Svoboda, D. S., Azzi, J., MacLaurin, J. G., Meghaizel, C., Sesaki, H., Lagace, D. C., Germain, M., Harper, M.-E., et al. (2016). Mitochondrial Dynamics Impacts Stem Cell Identity and Fate Decisions by Regulating a Nuclear Transcriptional Program. Cell Stem Cell 19, 232-247.
Kochanek, D. M., and Wells, D. G. (2013). CPEB1 Regulates the Expression of MTDH/AEG-1 and Glioblastoma Cell Migration. Mol. Cancer Res. 11, 149-160.
Kuang, S., Gillespie, M. A., and Rudnicki, M. A. (2008). Niche regulation of muscle satellite cell self-renewal and differentiation. Cell Stem Cell 2, 22-31.
Lesnik, C., Golani-Armon, A., and Arava, Y. (2015). Localized translation near the mitochondrial outer membrane: An update. RNA Biol. 12, 801-809.
Li, L., Rozo, M., Yue, S., Zheng, X., Tan, F., Lepper, C., and Fan, C.-M. (2019). Muscle stem cell renewal suppressed by GasI can be reversed by GDNF in mice. Nat. Metab. 1, 985-995.
Li, Q., Gao, Z., Chen, Y., and Guan, M.-X. (2017). The role of mitochondria in osteogenic, adipogenic and chondrogenic differentiation of mesenchymal stem cells. Protein Cell 8, 439-445.
Liao, Y., Smyth, G. K., and Shi, W. (2014). featureCounts: an efficient general purpose program for assigning sequence reads to genomic features. Bioinforma. Oxf. Engl. 30, 923-930.
Lisowski, P., Kannan, P., Mlody, B., and Prigione, A. (2018). Mitochondria and the dynamic control of stem cell homeostasis. EMBO Rep. 19.
Liu, L., Cheung, T. H., Charville, G. W., and Rando, T. A. (2015). Isolation of skeletal muscle stem cells by fluorescence-activated cell sorting. Nat. Protoc. 10, 1612-1624.
Liu, L., Charville, G. W., Cheung, T. H., Yoo, B., Santos, P. J., Schroeder, M., and Rando, T. A. (2018). Impaired Notch Signaling Leads to a Decrease in p53 Activity and Mitotic Catastrophe in Aged Muscle Stem Cells. Cell Stem Cell 23, 544-556.e4.
Love, M. I., Huber, W., and Anders, S. (2014). Moderated estimation of fold change and dispersion for RNA-seq data with DESeq2. Genome Biol. 15, 550-574.
Lu, R., Zhou, Z., Yu, W., Xia, Y., and Zhi, X. (2017a). CPEB4 promotes cell migration and invasion via upregulating Vimentin expression in breast cancer. Biochem. Biophys. Res. Commun. 489, 135-141.
Lu, W.-H., Yeh, N.-H., and Huang, Y.-S. (2017b). CPEB2 Activates GRASP1 mRNA Translation and Promotes AMPA Receptor Surface Expression, Long-Term Potentiation, and Memory. Cell Rep. 21, 1783-1794.
Maillo, C., Martin, J., Sebastián, D., Hernández-Alvarez, M., García-Rocha, M., Reina, O., Zorzano, A., Fernandez, M., and Méndez, R. (2017). Circadian- and UPR-dependent control of CPEB4 mediates a translational response to counteract hepatic steatosis under ER stress. Nat. Cell Biol. 19, 94-105.
Martinez-Reyes, I., Diebold, L. P., Kong, H., Schieber, M., Huang, H., Hensley, C. T., Mehta, M. M., Wang, T., Santos, J. H., Woychik, R., et al. (2016). TCA Cycle and Mitochondrial Membrane Potential Are Necessary for Diverse Biological Functions. Mol. Cell 61, 199-209.
Mejia-Ramirez, E., and Florian, M. C. (2020). Understanding intrinsic hematopoietic stem cell aging. Haematologica 105, 22-37.
Mejias, M., Gallego, J., Naranjo-Suarez, S., Ramirez, M., Pell, N., Manzano, A., Suner, C., Bartrons, R., Mendez, R., and Fernandez, M. (2020). CPEB4 Increases Expression of PFKFB3 to Induce Glycolysis and Activate Mouse and Human Hepatic Stellate Cells, Promoting Liver Fibrosis. Gastroenterology 159, 273-288.
Mohrin, M., Shin, J., Liu, Y., Brown, K., Luo, H., Xi, Y., Haynes, C. M., and Chen, D. (2015). Stem cell aging. A mitochondrial UPR-mediated metabolic checkpoint regulates hematopoietic stem cell aging. Science 347, 1374-1377.
Motohashi, N., and Asakura, A. (2014). Muscle satellite cell heterogeneity and self-renewal. Front. Cell Dev. Biol. 2.
Murphy, M. M., Lawson, J. A., Mathew, S. J., Hutcheson, D. A., and Kardon, G. (2011). Satellite cells, connective tissue fibroblasts and their interactions are crucial for muscle regeneration. Dev. Camb. Engl. 138, 3625-3637.
Nishijo, K., Hosoyama, T., Bjornson, C. R. R., Schaffer, B. S., Prajapati, S. I., Bahadur, A. N., Hansen, M. S., Blandford, M. C., McCleish, A. T., Rubin, B. P., et al. (2009). Biomarker system for studying muscle, stem cells, and cancer in vivo. FASEB J. Off. Publ. Fed. Am. Soc. Exp. Biol. 23, 2681-2690.
Novoa, I., Gallego, J., Ferreira, P. G., and Méndez, R. (2010). Mitotic cell-cycle progression is regulated by CPEB1 and CPEB4-dependent translational control. Nat. Cell Biol. 12, 447-456.
Papa, L., Djedaini, M., and Hoffman, R. (2019). Mitochondrial Role in Stemness and Differentiation of Hematopoietic Stem Cells. Stem Cells Int. 2019, e4067162.
Parras, A., Anta, H., Santos-Galindo, M., Swarup, V., Elorza, A., Nieto-González, J. L., Picó, S., Hernández, I. H., Diaz-Hernández, J. I., Belloc, E., et al. (2018). Autism-like phenotype and risk gene mRNA deadenylation by CPEB4 mis-splicing. Nature 560, 441-446.
Payne, B. A. I., and Chinnery, P. F. (2015). Mitochondrial dysfunction in aging: Much progress but many unresolved questions. Biochim. Biophys. Acta BBA—Bioenerg. 1847, 1347-1353.
Piqué, M., López, J. M., Foissac, S., Guigó, R., and Méndez, R. (2008). A combinatorial code for CPE-mediated translational control. Cell 132, 434-448.
Popow, J., Alleaume, A.-M., Curk, T., Schwarzl, T., Sauer, S., and Hentze, M. W. (2015). FASTKD2 is an RNA-binding protein required for mitochondrial RNA processing and translation. RNA 21, 1873-1884.
Rando, T. A. (2006). Stem cells, ageing and the quest for immortality. Nature 441, 1080-1086.
Rando, T. A., and Chang, H. Y. (2012). Aging, rejuvenation, and epigenetic reprogramming: resetting the aging clock. Cell 148, 46-57.
Raudvere, U., Kolberg, L., Kuzmin, I., Arak, T., Adler, P., Peterson, H., and Vilo, J. (2019). g:Profiler: a web server for functional enrichment analysis and conversions of gene lists (2019 update). Nucleic Acids Res. 47, 191-198.
Richter, J. D. (2007). CPEB: a life in translation. Trends Biochem. Sci. 32, 279-285. Ryall, J. G., Dell'Orso, S., Derfoul, A., Juan, A., Zare, H., Feng, X., Clermont, D., Koulnis, M., Gutierrez-Cruz, G., Fulco, M., et al. (2015). The NAD(+)-dependent SIRT1 deacetylase translates a metabolic switch into regulatory epigenetics in skeletal muscle stem cells. Cell Stem Cell 16, 171-183.
Sala, D., Cunningham, T. J., Stec, M. J., Etxaniz, U., Nicoletti, C., Dall'Agnese, A., Puri, P. L., Duester, G., Latella, L., and Sacco, A. (2019). The Stat3-Fam3a axis promotes muscle stem cell myogenic lineage progression by inducing mitochondrial respiration. Nat. Commun. 10, 1796.
Schultz, M. B., and Sinclair, D. A. (2016). When stem cells grow old: phenotypes and mechanisms of stem cell aging. Dev. Camb. Engl. 143, 3-14.
Shi, X., and Garry, D. J. (2006). Muscle stem cells in development, regeneration, and disease. Genes Dev. 20, 1692-1708.
Shin, J., Salameh, J. S., and Richter, J. D. (2016). Impaired neurodevelopment by the low complexity domain of CPEB4 reveals a convergent pathway with neurodegeneration. Sci. Rep. 6, 29395.
So, W.-K., and Cheung, T. H. (2018). Molecular Regulation of Cellular Quiescence: A Perspective from Adult Stem Cells and Its Niches. Methods Mol. Biol. Clifton NJ 1686, 1-25.
Sousa-Victor, P., Gutarra, S., García-Prat, L., Rodriguez-Ubreva, J., Ortet, L., Ruiz-Bonilla, V., Jardi, M., Ballestar, E., González, S., Serrano, A. L., et al. (2014). Geriatric muscle stem cells switch reversible quiescence into senescence. Nature 506, 316-321.
Stoll, E. A., Cheung, W., Mikheev, A. M., Sweet, I. R., Bielas, J. H., Zhang, J., Rostomily, R. C., and Homer, P. J. (2011). Aging neural progenitor cells have decreased mitochondrial content and lower oxidative metabolism. J. Biol. Chem. 286, 38592-38601.
Sun, N., Youle, R. J., and Finkel, T. (2016). The Mitochondrial Basis of Aging. Mol. Cell 61, 654-666.
Teperino, R., Schoonjans, K., and Auwerx, J. (2010). Histone methyl transferases and demethylases; can they link metabolism and transcription? Cell Metab. 12, 321-327.
Urbán, N., and Cheung, T. H. (2021). Stem cell quiescence: the challenging path to activation. Development 148.
Wallace, D. C., Fan, W., and Procaccio, V. (2010). Mitochondrial Energetics and Therapeutics. Annu. Rev. Pathol. 5, 297-348.
Yin, H., Price, F., and Rudnicki, M. A. (2013). Satellite Cells and the Muscle Stem Cell Niche. Physiol. Rev. 93, 23-67.
Yue, L., and Cheung, T. H. (2020). Protocol for Isolation and Characterization of In Situ Fixed Quiescent Muscle Stem Cells. STAR Protoc. 1, 100128-100144.
Yue, L., Wan, R., Luan, S., Zeng, W., and Cheung, T. H. (2020). Dek Modulates Global Intron Retention during Muscle Stem Cells Quiescence Exit. Dev. Cell 53, 661-676.
Zammit, P. S., Partridge, T. A., and Yablonka-Reuveni, Z. (2006). The Skeletal Muscle Satellite Cell: The Stem Cell That Came in From the Cold. J. Histochem. Cytochem. 54, 1177-1191.
Zhang, H., Ryu, D., Wu, Y., Gariani, K., Wang, X., Luan, P., D'Amico, D., Ropelle, E. R., Lutolf, M. P., Aebersold, R., et al. (2016). NAD+ repletion improves mitochondrial and stem cell function and enhances life span in mice. Science 352, 1436-1443.
Zhang, H., Zou, C., Qiu, Z., E, F., Li, Q., Chen, M., Wang, D., Tan, Q., Yin, W., Matunda, C., et al. (2020). CPEB3-mediated MTDH mRNA translational suppression restrains hepatocellular carcinoma progression. Cell Death Dis. 11, 1-17.
Zhu, W., Smith, J. W., and Huang, C.-M. (2010). Mass spectrometry-based label-free quantitative proteomics. J. Biomed. Biotechnol. 2010, 840518-840526.

SEQUENCE LISTING

SEQ ID NO: 1 Human CPEB4 coding sequence, GenBank Accession No. NM_030627.4
ATGGGGGATTACGGGTTTGGAGTGCTAGTGCAAAGCAATACTGGGAATAAATCTGCTTTTCCAGTCAG
ATTCCATCCACATCTGCAGCCTCCACACCATCACCAAAATGCCACCCCCAGCCCTGCTGCTTTTATAA
ATAATAACACAGCTGCCAATGGCAGCAGTGCTGGGTCAGCTTGGCTTTTTCCTGCTCCAGCTACCCAT
AACATTCAGGATGAGATCTTGGGGTCAGAAAAAGCAAAAAGTCAGCAACAGGAACAGCAAGACCCCTT
AGAAAAGCAGCAGCTTTCCCCAAGTCCAGGTCAGGAAGCTGGAATACTGCCTGAAACAGAGAAGGCAA
AATCAGAAGAAAATCAAGGGGACAATTCTTCGGAAAATGGCAATGGGAAGGAGAAAATAAGGATCGAA
TCTCCAGTGTTGACAGGGTTTGATTATCAAGAAGCCACTGGGCTAGGTACTTCAACCCAACCCTTGAC
ATCTAGCGCATCGTCTCTTACTGGTTTCAGTAACTGGTCAGCAGCGATAGCGCCTTCCTCCTCTACAA
TAATCAATGAAGATGCAAGTTTCTTTCACCAGGGAGGGGTCCCTGCTGCTTCGGCTAATAACGGTGCT
CTGTTGTTTCAAAATTTCCCCCATCATGTCAGCCCTGGCTTTGGAGGCAGCTTCTCTCCTCAGATCGG
GCCTCTCTCACAGCACCACCCACATCACCCTCATTTCCAGCATCATCACAGCCAGCATCAGCAGCAAA
GGAGGTCTCCTGCCAGTCCCCATCCCCCACCCTTCACACATAGAAATGCTGCTTTTAACCAGCTGCCT
CATTTGGCGAATAATCTTAACAAACCCCCCTCTCCGTGGAGCAGCTACCAGAGTCCGTCACCAACACC
CTCCTCTTCCTGGAGCCCGGGCGGTGGTGGATATGGTGGCTGGGGAGGTTCCCAAGGCCGAGATCACC
GCAGAGGGCTGAATGGTGGAATAACGCCCCTGAACTCCATCTCGCCTTTGAAGAAAAATTTTGCAAGC
AATCATATTCAGCTCCAGAAGTATGCTCGCCCCAGCTCTGCCTTTGCACCTAAATCCTGGATGGAAGA
TAGCTTGAACAGGGCTGACAACATTTTTCCTTTTCCGGATCGCCCCAGGACATTCGACATGCACTCAC
TGGAGAGTTCACTCATTGACATAATGAGAGCTGAAAATGATACCATTAAAGGTCGTCTAAACTATTCA
TATCCAGGATCCGATAGCTCTCTGCTTATTAATGCAAGGACATATGGGCGAAGGAGAGGTCAGTCTTC
ACTGTTTCCAATGGAAGATGGATTCTTGGATGATGGCCGTGGGGATCAGCCTCTTCATAGTGGCCTGG
GTTCACCTCACTGCTTCAGTCACCAGAATGGGGAAAGAGTGGAACGATATTCTCGAAAGGTGTTTGTA
GGCGGATTGCCTCCAGACATTGATGAAGATGAGATCACAGCTAGTTTTCGTCGCTTTGGCCCTCTGAT
TGTGGATTGGCCTCATAAAGCTGAGAGCAAATCCTATTTTCCTCCTAAAGGCTATGCATTCCTGCTGT
TTCAAGATGAAAGCTCTGTGCAGGCTCTCATTGATGCATGCATTGAAGAAGATGGAAAACTCTACCTT
TGTGTATCAAGTCCCACTATCAAGGATAAGCCAGTCCAGATTCGGCCTTGGAATCTCAGTGACAGTGA
CTTTGTGATGGATGGTTCACAGCCACTTGACCCACGAAAAACTATATTTGTTGGTGGTGTTCCTCGAC
CATTACGAGCTGTGGAGCTTGCGATGATAATGGATCGGCTATATGGAGGTGTGTGCTACGCTGGGATT
GATACCGACCCTGAGCTAAAATACCCAAAAGGAGCTGGGAGAGTTGCGTTCTCTAATCAACAGAGTTA
CATAGCTGCTATCAGTGCCCGCTTTGTTCAGCTGCAGCATGGAGAGATAGATAAACGGGTGGAAGTTA
AGCCATATGTCTTGGATGATCAGCTGTGTGATGAATGTCAGGGGGCCCGTTGTGGGGGGAAATTTGCT
CCATTTTTCTGTGCTAATGTTACCTGTCTGCAGTATTACTGTGAATATTGCTGGGCTGCTATCCATTC
TCGTGCTGGCAGGGAATTCCACAAGCCCCTGGTGAAGGAAGGCGGTGACCGCCCTCGGCATATTTCAT
TCCGCTGGAACTAA
SEQ ID NO: 2 Amino acid sequence of human CPEB4 protein, GenBank Accession No.
NP_085130.2
MGDYGFGVLVQSNTGNKSAFPVRFHPHLQPPHHHQNATPSPAAFINNNTAANGSSAGSAWLFPAPATH
NIQDEILGSEKAKSQQQEQQDPLEKQQLSPSPGQEAGILPETEKAKSEENQGDNSSENGNGKEKIRIE
SPVLTGFDYQEATGLGTSTQPLTSSASSLTGFSNWSAAIAPSSSTIINEDASFFHQGGVPAASANNGA
LLFQNFPHHVSPGFGGSFSPQIGPLSQHHPHHPHFQHHHSQHQQQRRSPASPHPPPFTHRNAAFNQLP
HLANNLNKPPSPWSSYQSPSPTPSSSWSPGGGGYGGWGGSQGRDHRRGLNGGITPLNSISPLKKNFAS
NHIQLQKYARPSSAFAPKSWMEDSLNRADNIFPFPDRPRTFDMHSLESSLIDIMRAENDTIKGRLNYS
YPGSDSSLLINARTYGRRRGQSSLFPMEDGFLDDGRGDQPLHSGLGSPHCFSHQNGERVERYSRKVFV
GGLPPDIDEDEITASFRRFGPLIVDWPHKAESKSYFPPKGYAFLLFQDESSVQALIDACIEEDGKLYL
CVSSPTIKDKPVQIRPWNLSDSDFVMDGSQPLDPRKTIFVGGVPRPLRAVELAMIMDRLYGGVCYAGI
DTDPELKYPKGAGRVAFSNQQSYIAAISARFVQLQHGEIDKRVEVKPYVLDDQLCDECQGARCGGKFA
PFFCANVTCLQYYCEYCWAAIHSRAGREFHKPLVKEGGDRPRHISFRWN
SEQ ID NO: 3 Human CPEB4 isoform b coding sequence (variant 2), Genbank
Accession No: NM_001308189.2
ATGGGGGATTACGGGTTTGGAGTGCTAGTGCAAAGCAATACTGGGAATAAATCTGCTTTTCCAGTCAG
ATTCCATCCACATCTGCAGCCTCCACACCATCACCAAAATGCCACCCCCAGCCCTGCTGCTTTTATAA
ATAATAACACAGCTGCCAATGGCAGCAGTGCTGGGTCAGCTTGGCTTTTTCCTGCTCCAGCTACCCAT
AACATTCAGGATGAGATCTTGGGGTCAGAAAAAGCAAAAAGTCAGCAACAGGAACAGCAAGACCCCTT
AGAAAAGCAGCAGCTTTCCCCAAGTCCAGGTCAGGAAGCTGGAATACTGCCTGAAACAGAGAAGGCAA
AATCAGAAGAAAATCAAGGGGACAATTCTTCGGAAAATGGCAATGGGAAGGAGAAAATAAGGATCGAA
TCTCCAGTGTTGACAGGGTTTGATTATCAAGAAGCCACTGGGCTAGGTACTTCAACCCAACCCTTGAC
ATCTAGCGCATCGTCTCTTACTGGTTTCAGTAACTGGTCAGCAGCGATAGCGCCTTCCTCCTCTACAA
TAATCAATGAAGATGCAAGTTTCTTTCACCAGGGAGGGGTCCCTGCTGCTTCGGCTAATAACGGTGCT
CTGTTGTTTCAAAATTTCCCCCATCATGTCAGCCCTGGCTTTGGAGGCAGCTTCTCTCCTCAGATCGG
GCCTCTCTCACAGCACCACCCACATCACCCTCATTTCCAGCATCATCACAGCCAGCATCAGCAGCAAA
GGAGGTCTCCTGCCAGTCCCCATCCCCCACCCTTCACACATAGAAATGCTGCTTTTAACCAGCTGCCT
CATTTGGCGAATAATCTTAACAAACCCCCCTCTCCGTGGAGCAGCTACCAGAGTCCGTCACCAACACC
CTCCTCTTCCTGGAGCCCGGGCGGTGGTGGATATGGTGGCTGGGGAGGTTCCCAAGGCCGAGATCACC
GCAGAGGGCTGAATGGTGGAATAACGCCCCTGAACTCCATCTCGCCTTTGAAGAAAAATTTTGCAAGC
AATCATATTCAGCTCCAGAAGTATGCTCGCCCCAGCTCTGCCTTTGCACCTAAATCCTGGATGGAAGA
TAGCTTGAACAGGGCTGACAACATTTTTCCTTTTCCGGATCGCCCCAGGACATTCGACATGCACTCAC
TGGAGAGTTCACTCATTGACATAATGAGAGCTGAAAATGATACCATTAAAGCAAGGACATATGGGCGA
AGGAGAGGTCAGTCTTCACTGTTTCCAATGGAAGATGGATTCTTGGATGATGGCCGTGGGGATCAGCC
TCTTCATAGTGGCCTGGGTTCACCTCACTGCTTCAGTCACCAGAATGGGGAAAGAGTGGAACGATATT
CTCGAAAGGTGTTTGTAGGCGGATTGCCTCCAGACATTGATGAAGATGAGATCACAGCTAGTTTTCGT
CGCTTTGGCCCTCTGATTGTGGATTGGCCTCATAAAGCTGAGAGCAAATCCTATTTTCCTCCTAAAGG
CTATGCATTCCTGCTGTTTCAAGATGAAAGCTCTGTGCAGGCTCTCATTGATGCATGCATTGAAGAAG
ATGGAAAACTCTACCTTTGTGTATCAAGTCCCACTATCAAGGATAAGCCAGTCCAGATTCGGCCTTGG
AATCTCAGTGACAGTGACTTTGTGATGGATGGTTCACAGCCACTTGACCCACGAAAAACTATATTTGT
TGGTGGTGTTCCTCGACCATTACGAGCTGTGGAGCTTGCGATGATAATGGATCGGCTATATGGAGGTG
TGTGCTACGCTGGGATTGATACCGACCCTGAGCTAAAATACCCAAAAGGAGCTGGGAGAGTTGCGTTC
TCTAATCAACAGAGTTACATAGCTGCTATCAGTGCCCGCTTTGTTCAGCTGCAGCATGGAGAGATAGA
TAAACGGGTGGAAGTTAAGCCATATGTCTTGGATGATCAGCTGTGTGATGAATGTCAGGGGGCCCGTT
GTGGGGGGAAATTTGCTCCATTTTTCTGTGCTAATGTTACCTGTCTGCAGTATTACTGTGAATATTGC
TGGGCTGCTATCCATTCTCGTGCTGGCAGGGAATTCCACAAGCCCCTGGTGAAGGAAGGCGGTGACCG
CCCTCGGCATATTTCATTCCGCTGGAACTAA
SEQ ID NO: 4 Amino acid sequence of Human CPEB4 isoform b (variant 2), Genbank
Accession No: NP_001295118.1
MGDYGFGVLVQSNTGNKSAFPVRFHPHLQPPHHHQNATPSPAAFINNNTAANGSSAGSAWLFPAPATH
NIQDEILGSEKAKSQQQEQQDPLEKQQLSPSPGQEAGILPETEKAKSEENQGDNSSENGNGKEKIRIE
SPVLTGFDYQEATGLGTSTQPLTSSASSLTGFSNWSAAIAPSSSTIINEDASFFHQGGVPAASANNGA
LLFQNFPHHVSPGFGGSFSPQIGPLSQHHPHHPHFQHHHSQHQQQRRSPASPHPPPFTHRNAAFNQLP
HLANNLNKPPSPWSSYQSPSPTPSSSWSPGGGGYGGWGGSQGRDHRRGLNGGITPLNSISPLKKNFAS
NHIQLQKYARPSSAFAPKSWMEDSLNRADNIFPFPDRPRTFDMHSLESSLIDIMRAENDTIKARTYGR
RRGQSSLFPMEDGFLDDGRGDQPLHSGLGSPHCFSHQNGERVERYSRKVFVGGLPPDIDEDEITASFR
RFGPLIVDWPHKAESKSYFPPKGYAFLLFQDESSVQALIDACIEEDGKLYLCVSSPTIKDKPVQIRPW
NLSDSDFVMDGSQPLDPRKTIFVGGVPRPLRAVELAMIMDRLYGGVCYAGIDTDPELKYPKGAGRVAF
SNQQSYIAAISARFVQLQHGEIDKRVEVKPYVLDDQLCDECQGARCGGKFAPFFCANVTCLQYYCEYC
WAAIHSRAGREFHKPLVKEGGDRPRHISFRWN
SEQ ID NO: 5 Human CPEB4 isoform c coding sequence (variant 3), Genbank Accession
No: NM_001308191.2
ATGGGGGATTACGGGTTTGGAGTGCTAGTGCAAAGCAATACTGGGAATAAATCTGCTTTTCCAGTCAG
ATTCCATCCACATCTGCAGCCTCCACACCATCACCAAAATGCCACCCCCAGCCCTGCTGCTTTTATAA
ATAATAACACAGCTGCCAATGGCAGCAGTGCTGGGTCAGCTTGGCTTTTTCCTGCTCCAGCTACCCAT
AACATTCAGGATGAGATCTTGGGGTCAGAAAAAGCAAAAAGTCAGCAACAGGAACAGCAAGACCCCTT
AGAAAAGCAGCAGCTTTCCCCAAGTCCAGGTCAGGAAGCTGGAATACTGCCTGAAACAGAGAAGGCAA
AATCAGAAGAAAATCAAGGGGACAATTCTTCGGAAAATGGCAATGGGAAGGAGAAAATAAGGATCGAA
TCTCCAGTGTTGACAGGGTTTGATTATCAAGAAGCCACTGGGCTAGGTACTTCAACCCAACCCTTGAC
ATCTAGCGCATCGTCTCTTACTGGTTTCAGTAACTGGTCAGCAGCGATAGCGCCTTCCTCCTCTACAA
TAATCAATGAAGATGCAAGTTTCTTTCACCAGGGAGGGGTCCCTGCTGCTTCGGCTAATAACGGTGCT
CTGTTGTTTCAAAATTTCCCCCATCATGTCAGCCCTGGCTTTGGAGGCAGCTTCTCTCCTCAGATCGG
GCCTCTCTCACAGCACCACCCACATCACCCTCATTTCCAGCATCATCACAGCCAGCATCAGCAGCAAA
GGAGGTCTCCTGCCAGTCCCCATCCCCCACCCTTCACACATAGAAATGCTGCTTTTAACCAGCTGCCT
CATTTGGCGAATAATCTTAACAAACCCCCCTCTCCGTGGAGCAGCTACCAGAGTCCGTCACCAACACC
CTCCTCTTCCTGGAGCCCGGGCGGTGGTGGATATGGTGGCTGGGGAGGTTCCCAAGGCCGAGATCACC
GCAGAGGGCTGAATGGTGGAATAACGCCCCTGAACTCCATCTCGCCTTTGAAGAAAAATTTTGCAAGC
AATCATATTCAGCTCCAGAAGTATGCTCGCCCCAGCTCTGCCTTTGCACCTAAATCCTGGATGGAAGA
TAGCTTGAACAGGGCTGACAACATTTTTCCTTTTCCGGATCGCCCCAGGACATTCGACATGCACTCAC
TGGAGAGTTCACTCATTGACATAATGAGAGCTGAAAATGATACCATTAAAGGTCAGTCTTCACTGTTT
CCAATGGAAGATGGATTCTTGGATGATGGCCGTGGGGATCAGCCTCTTCATAGTGGCCTGGGTTCACC
TCACTGCTTCAGTCACCAGAATGGGGAAAGAGTGGAACGATATTCTCGAAAGGTGTTTGTAGGCGGAT
TGCCTCCAGACATTGATGAAGATGAGATCACAGCTAGTTTTCGTCGCTTTGGCCCTCTGATTGTGGAT
TGGCCTCATAAAGCTGAGAGCAAATCCTATTTTCCTCCTAAAGGCTATGCATTCCTGCTGTTTCAAGA
TGAAAGCTCTGTGCAGGCTCTCATTGATGCATGCATTGAAGAAGATGGAAAACTCTACCTTTGTGTAT
CAAGTCCCACTATCAAGGATAAGCCAGTCCAGATTCGGCCTTGGAATCTCAGTGACAGTGACTTTGTG
ATGGATGGTTCACAGCCACTTGACCCACGAAAAACTATATTTGTTGGTGGTGTTCCTCGACCATTACG
AGCTGTGGAGCTTGCGATGATAATGGATCGGCTATATGGAGGTGTGTGCTACGCTGGGATTGATACCG
ACCCTGAGCTAAAATACCCAAAAGGAGCTGGGAGAGTTGCGTTCTCTAATCAACAGAGTTACATAGCT
GCTATCAGTGCCCGCTTTGTTCAGCTGCAGCATGGAGAGATAGATAAACGGGTGGAAGTTAAGCCATA
TGTCTTGGATGATCAGCTGTGTGATGAATGTCAGGGGGCCCGTTGTGGGGGGAAATTTGCTCCATTTT
TCTGTGCTAATGTTACCTGTCTGCAGTATTACTGTGAATATTGCTGGGCTGCTATCCATTCTCGTGCT
GGCAGGGAATTCCACAAGCCCCTGGTGAAGGAAGGCGGTGACCGCCCTCGGCATATTTCATTCCGCTG
GAACTAA
SEQ ID NO: 6 Amino acid sequence of Human CPEB4 isoform c (variant 3), Genbank
Accession No: NM_001295120.1
MGDYGFGVLVQSNTGNKSAFPVRFHPHLQPPHHHQNATPSPAAFINNNTAANGSSAGSAWLFPAPATH
NIQDEILGSEKAKSQQQEQQDPLEKQQLSPSPGQEAGILPETEKAKSEENQGDNSSENGNGKEKIRIE
SPVLTGFDYQEATGLGTSTQPLTSSASSLTGFSNWSAAIAPSSSTIINEDASFFHQGGVPAASANNGA
LLFQNFPHHVSPGFGGSFSPQIGPLSQHHPHHPHFQHHHSQHQQQRRSPASPHPPPFTHRNAAFNQLP
HLANNLNKPPSPWSSYQSPSPTPSSSWSPGGGGYGGWGGSQGRDHRRGLNGGITPLNSISPLKKNFAS
NHIQLQKYARPSSAFAPKSWMEDSLNRADNIFPFPDRPRTFDMHSLESSLIDIMRAENDTIKGQSSLF
PMEDGFLDDGRGDQPLHSGLGSPHCFSHQNGERVERYSRKVFVGGLPPDIDEDEITASFRRFGPLIVD
WPHKAESKSYFPPKGYAFLLFQDESSVQALIDACIEEDGKLYLCVSSPTIKDKPVQIRPWNLSDSDFV
MDGSQPLDPRKTIFVGGVPRPLRAVELAMIMDRLYGGVCYAGIDTDPELKYPKGAGRVAFSNQQSYIA
AISARFVQLQHGEIDKRVEVKPYVLDDQLCDECQGARCGGKFAPFFCANVTCLQYYCEYCWAAIHSRA
GREFHKPLVKEGGDRPRHISFRWN
SEQ ID NO: 7 Human CPEB4 isoform d coding sequence (variant 4), Genbank Accession
No: NM_001308192.2
ATGCACTCACTGGAGAGTTCACTCATTGACATAATGAGAGCTGAAAATGATACCATTAAAGGTCGTCT
AAACTATTCATATCCAGGATCCGATAGCTCTCTGCTTATTAATGGTCAGTCTTCACTGTTTCCAATGG
AAGATGGATTCTTGGATGATGGCCGTGGGGATCAGCCTCTTCATAGTGGCCTGGGTTCACCTCACTGC
TTCAGTCACCAGAATGGGGAAAGAGTGGAACGATATTCTCGAAAGGTGTTTGTAGGCGGATTGCCTCC
AGACATTGATGAAGATGAGATCACAGCTAGTTTTCGTCGCTTTGGCCCTCTGATTGTGGATTGGCCTC
ATAAAGCTGAGAGCAAATCCTATTTTCCTCCTAAAGGCTATGCATTCCTGCTGTTTCAAGATGAAAGC
TCTGTGCAGGCTCTCATTGATGCATGCATTGAAGAAGATGGAAAACTCTACCTTTGTGTATCAAGTCC
CACTATCAAGGATAAGCCAGTCCAGATTCGGCCTTGGAATCTCAGTGACAGTGACTTTGTGATGGATG
GTTCACAGCCACTTGACCCACGAAAAACTATATTTGTTGGTGGTGTTCCTCGACCATTACGAGCTGTG
GAGCTTGCGATGATAATGGATCGGCTATATGGAGGTGTGTGCTACGCTGGGATTGATACCGACCCTGA
GCTAAAATACCCAAAAGGAGCTGGGAGAGTTGCGTTCTCTAATCAACAGAGTTACATAGCTGCTATCA
GTGCCCGCTTTGTTCAGCTGCAGCATGGAGAGATAGATAAACGGGTGGAAGTTAAGCCATATGTCTTG
GATGATCAGCTGTGTGATGAATGTCAGGGGGCCCGTTGTGGGGGGAAATTTGCTCCATTTTTCTGTGC
TAATGTTACCTGTCTGCAGTATTACTGTGAATATTGCTGGGCTGCTATCCATTCTCGTGCTGGCAGGG
AATTCCACAAGCCCCTGGTGAAGGAAGGCGGTGACCGCCCTCGGCATATTTCATTCCGCTGGAACTAA
SEQ ID NO: 8 Amino acid sequence of Human CPEB4 isoform d (variant 4), Genbank
Accession No: NP_001295121.1
MHSLESSLIDIMRAENDTIKGRLNYSYPGSDSSLLINGQSSLFPMEDGFLDDGRGDQPLHSGLGSPHC
FSHQNGERVERYSRKVFVGGLPPDIDEDEITASFRRFGPLIVDWPHKAESKSYFPPKGYAFLLFQDES
SVQALIDACIEEDGKLYLCVSSPTIKDKPVQIRPWNLSDSDFVMDGSQPLDPRKTIFVGGVPRPLRAV
ELAMIMDRLYGGVCYAGIDTDPELKYPKGAGRVAFSNQQSYIAAISARFVQLQHGEIDKRVEVKPYVL
DDQLCDECQGARCGGKFAPFFCANVTCLQYYCEYCWAAIHSRAGREFHKPLVKEGGDRPRHISFRWN
SEQ ID NO: 9 Human CPEB4 isoform e coding sequence (variant 5), Genbank Accession
No: NM_001308193.2
ATGCACTCACTGGAGAGTTCACTCATTGACATAATGAGAGCTGAAAATGATACCATTAAAGGTCAGTC
TTCACTGTTTCCAATGGAAGATGGATTCTTGGATGATGGCCGTGGGGATCAGCCTCTTCATAGTGGCC
TGGGTTCACCTCACTGCTTCAGTCACCAGAATGGGGAAAGAGTGGAACGATATTCTCGAAAGGTGTTT
GTAGGCGGATTGCCTCCAGACATTGATGAAGATGAGATCACAGCTAGTTTTCGTCGCTTTGGCCCTCT
GATTGTGGATTGGCCTCATAAAGCTGAGAGCAAATCCTATTTTCCTCCTAAAGGCTATGCATTCCTGC
TGTTTCAAGATGAAAGCTCTGTGCAGGCTCTCATTGATGCATGCATTGAAGAAGATGGAAAACTCTAC
CTTTGTGTATCAAGTCCCACTATCAAGGATAAGCCAGTCCAGATTCGGCCTTGGAATCTCAGTGACAG
TGACTTTGTGATGGATGGTTCACAGCCACTTGACCCACGAAAAACTATATTTGTTGGTGGTGTTCCTC
GACCATTACGAGCTGTGGAGCTTGCGATGATAATGGATCGGCTATATGGAGGTGTGTGCTACGCTGGG
ATTGATACCGACCCTGAGCTAAAATACCCAAAAGGAGCTGGGAGAGTTGCGTTCTCTAATCAACAGAG
TTACATAGCTGCTATCAGTGCCCGCTTTGTTCAGCTGCAGCATGGAGAGATAGATAAACGGGTGGAAG
TTAAGCCATATGTCTTGGATGATCAGCTGTGTGATGAATGTCAGGGGGCCCGTTGTGGGGGGAAATTT
GCTCCATTTTTCTGTGCTAATGTTACCTGTCTGCAGTATTACTGTGAATATTGCTGGGCTGCTATCCA
TTCTCGTGCTGGCAGGGAATTCCACAAGCCCCTGGTGAAGGAAGGCGGTGACCGCCCTCGGCATATTT
CATTCCGCTGGAACTAA
SEQ ID NO: 10 Amino acid sequence of Human CPEB4 isoform e (variant 5), Genbank
Accession No: NP_001295122.1
MHSLESSLIDIMRAENDTIKGQSSLFPMEDGFLDDGRGDQPLHSGLGSPHCFSHQNGERVERYSRKVF
VGGLPPDIDEDEITASFRRFGPLIVDWPHKAESKSYFPPKGYAFLLFQDESSVQALIDACIEEDGKLY
LCVSSPTIKDKPVQIRPWNLSDSDFVMDGSQPLDPRKTIFVGGVPRPLRAVELAMIMDRLYGGVCYAG
IDTDPELKYPKGAGRVAFSNQQSYIAAISARFVQLQHGEIDKRVEVKPYVLDDQLCDECQGARCGGKF
APFFCANVTCLQYYCEYCWAAIHSRAGREFHKPLVKEGGDRPRHISFRWN

1. A method for rejuvenating a cell, comprising step (1) increasing CPEB4 expression and/or activity in the cell.

2. The method of claim 1, wherein the cell is a muscle cell or muscle stem cell.

3. The method of claim 1, wherein step (1) comprises introducing into the cell a nucleic acid encoding CPEB4 protein such that an effective amount of CPEB4 protein is expressed in the cell.

4. The method of claim 1, wherein the cell is within a human body.

5. The method of claim 4, wherein step (1) comprises administering to the human an expression vector comprising a polynucleotide sequence encoding CPEB4 protein.

6. The method of claim 5, wherein the expression vector is administered by subcutaneous, intramuscular, intravenous, or intraperitoneal injection, or by oral administration.

7. The method of claim 1, wherein step (1) is performed ex vivo prior to the cell being reintroduced back into a human body.

8. The method of claim 6, wherein the cell is a muscle stem cell and wherein the cell is reintroduced to a muscle injury site.

9. The method of claim 2, wherein the expression vector comprises a muscle cell-specific promoter operably linked to a polynucleotide sequence encoding CPEB4 protein.

10. The method of claim 9, wherein the muscle cell-specific promoter is a Pax7 promoter.

11. A rejuvenated cell produced by the method of claim 1.

12. The cell of claim 11, which is a muscle cell or a muscle stem cell.

13. A composition comprising the cell of claim 11 and a physiologically acceptable excipient.

14. The composition of claim 13, which is formulated for subcutaneous, intramuscular, intravenous, or intraperitoneal injection, or for oral administration.

15. The composition of claim 12, wherein the cell is a muscle cell or a muscle stem cell.

16. A kit comprising a container containing (a) a composition comprising an expression vector, which comprises a polynucleotide sequence encoding CPEB4 protein; or (b) the composition of claim 13.

17. The kit of claim 16, wherein the expression vector comprises a muscle cell-specific promoter operably linked to the polynucleotide sequence encoding CPEB4 protein.

18. The kit of claim 17, wherein the cell is a muscle cell or a muscle stem cell.

19. A method for identifying a modulator of cell senescence, comprising:
(i) contacting a cell with a candidate compound;
(ii) determining CPEB4 protein level within the cell following step (i); and
(iii) identifying the compound as a promoter of cell senescence upon determining the CPEB4 protein level in step (ii) being lower than the CPEB4 protein level in the cell in the absence of the compound, and identifying the compound as an inhibitor of cell senescence upon determining the CPEB4 protein level in step (ii) being higher than the CPEB4 protein level in the cell in the absence of the compound.

20. The method of claim 19, wherein the cell is a muscle cell or a muscle stem cell.

21. A method for assessing cell senescence, comprising:
(a) determining CPEB4 protein level in a first cell;
(b) determining CPEB4 protein level in a second cell of the same kind as the first cell; and
(c) determining the first cell as being less senescent than the second cell upon determining the CPEB4 protein level is higher in the first cell than the CPEB4 protein level in the second cell.

22. The method of claim 21, wherein the first and second cells are muscle cells or muscle stem cells.

23. The method of claim 21, wherein the first and second cells are taken from two anatomic sites of one individual or from two different individuals.
